SEQUENCE LISTING (109 sequences)

SEQ ID NO: 1 - protein, 446 aa, Artificial Sequence
  ORF0657nI with amino terminus methionine
MAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTP

SEQ ID NO: 2 - protein, 645 aa, S. aureus
MNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 3 - protein, 569 aa, Artificial Sequence
  ORF0657nH with amino terminus methionine
MAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 4 - protein, 570 aa, Artificial Sequence
  ORF0657nH with amino terminus methionine-glycine
MGAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 5 - protein, 447 aa, Artificial Sequence
  ORF0657nH with amino terminus methionine-glycine
MGAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTP

SEQ ID NO: 6 - protein, 576 aa, Artificial Sequence
  ORF0657nH
AEETGVTNTEAQPKTEAVASPTTTTTEKAPEAKPVAKPVANAVSVSNKEVVAPTTETKEAKEVKAVKEVKAPKEAKEEKPAAKADNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDAFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKTNKKEQQDNSAKKETTPAMPSKPTTPPVEKESQKQDSQKDDNKQSPSVEKENDASSESGKDKMPVTKPAKAEVESSSTTPTKVVSTTQNVAKPTTASSETTKDVVQTSAGSSEAKDSAPLQKANIKNTNDGHTQSQNNKNTQENKAKS

SEQ ID NO: 7 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKGVTLATKPTKGEVESSSTTPTKVVSTTQNVAKPTTGSSKTTKDVVQTSAGSSEAKDSAPLQKANIKHTNDGHTQSQNNKNTQENKAKS

SEQ ID NO: 8 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 9 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 10 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQKVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 11 - protein, 565 aa, Artificial Sequence
  ORF0657nH
AEETGGTNTEAQPKTEAVASPTTTTEKAPEAKPVANAVSVSNKEVEAPTSETKEAKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSASSSEAKDSAPLQKANIKNTNDGHTQSQNNKNTQENKAKS

SEQ ID NO: 12 - protein, 566 aa, Artificial Sequence
  ORF0657nH
AEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSTTPTKVVSTTQNVAKPTASSKTTKDVVQTSAGSSEAKDSAPLQKANIKNTNDGHTQSQNNKNTQENKAKS

SEQ ID NO: 13 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGKVESSSTTPTKVVSTTQNVAKPTTASSKTTKDVVQTSAGSSEAKDSAPLQKANIKNTNDGHTQSQNNKNTQENKAKS

SEQ ID NO: 14 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPEIKDKDHSAPNSRPIDFEMKKKDGTQQFYHYASSVKPARVIFTDSKPEIELGLQSGQFWRKFEVYEGDKKLPIKLASYDTVKDYAYIRFSISNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVVKPTTASSKTTKDVVQTSAGSSEAKDSAPLQKANIKNTNDGHTQSQNNKNTQENKAKS

SEQ ID NO: 15 - protein, 564 aa, Artificial Sequence
  ORF0657nH
AEETGGTNTEAQPKTEAVASPTTTTEKAPEAKPVANAVSVSNKEVEAPTSETKEAKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTASSKTTKDVVQTSASSSEAKDSAPLQKANIKNTNDGHTQSQNNKNTQENKAKS

SEQ ID NO: 16 - protein, 565 aa, Artificial Sequence
  ORF0657nH
AEETGGTNTEAQPKTEAVASPTTTTEKAPEAKPVANAVSVSNKEVEAPTSETKEAKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVRVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKS

SEQ ID NO: 17 - protein, 568 aa, Artificial Sequence
  ORF0657nH
AEETGGTNTEAQPKTEALASPTTTTEKAPETKPVANAVSVSNKEVEAPTSETKEAKEVKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAINNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKS

SEQ ID NO: 18 - protein, 565 aa, Artificial Sequence
  ORF0657nH
AEETGGTNTEAQPKTEAVASPTTTTEKAPEAKPVANAVSVSNKEVEAPTSETKEAKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKPLPSVEKENDASSESGKDKTPATKPAKGEVESSSTTPTKVVSTTQNVAKPTTASSKTTKDVVQTSASSSEAKDSAPLQKANIKNTNDGHTQSQNNKNTQENKAKS

SEQ ID NO: 19 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKAVKPATKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKREATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKS

SEQ ID NO: 20 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKAVKPATKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKS

SEQ ID NO: 21 - protein, 576 aa, Artificial Sequence
  ORF0657nH
AEETGVTNTEAQPKTEAVASPTTTTTEKAPEAKPVAKPVANAVSVSNKEVVAPTTETKEAKEVKAVKEVKAPKEAKEEKPAAKADNNTYPILNQELREAIKNPAIKDKDHSAPNSRPIDFEMKKKDGTQQFYHYAGSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKTNKKEQQDNSAKKETTPAMPSKPTTPPVEKESQKQDSQKDDNKQSPGVEKENDASSESGKDKMPVTKPAKAEVESSSTTPTKVVSTTQNVAKPTTASSETTKDVVQTSAGSSEAKDSAPLQKANIKNTNDGHTQSQNNKNTQENKAKS

SEQ ID NO: 22 - protein, 576 aa, Artificial Sequence
  ORF0657nH
AEETGVTNTEAQPKTEAVASPTTTTTEKAPEAKPVAKPVANAVSVSNKEVVAPTTETKEAKEVKAVKEVKAPKEAKEEKPAAKADNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKTNKKEQQDNSAKKETTPAMPSKPTTPPVEKESQKQDSQKDDNKQSPSVEKENDASSESGKDKMPVTKPAKAEVESSSTTPTKVVSTTQNVAKPTTASSETTKDVVQTSAGSSEAKDSAPLQKANIKNTNDGHTQSQNNKNTQENKAKS

SEQ ID NO: 23 - protein, 568 aa, Artificial Sequence
  ORF0657nH
AEETGGTNTEAQPKTEAVASPSTTTEKAPEAKPVANAVSVSNKEVEAPTSETKEAKEVKEVKAPKETKEVKPATKADNNTYPILNQELREAIKNPAIKDKDHSAPNSRPIDFEMKKKDGTQQFYHYASSVKPARVIFTDSKPEIELGLQSGQFWRKFEVYEGNKKLPIKLVSYDTVKDYAYIRFSVSNGTKAVKIVSSTHFNNKEEKYDYTLMEFAQPIYNSAGKFKTEEDYKAEKLLSPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKSNKKEQQDNSAKKETTPATPSKPTTPPVEKESQKQDSQKDDNKQSPSVEKENDASSESGKDKTPTTKPAKAEVESSSTTPTKVVSTTQNVAKPTTASSETTIDVVQTSAGSSEAKDSAPLQKANIKNTNDGHTQSENNKNTQENKAKS

SEQ ID NO: 24 - protein, 568 aa, Artificial Sequence
  ORF0657nH
AEETGGTNTEAQPKTEAVASPSTTTEKAPEAKPVANAVSVSNKEVEAPTSETKEAKEVKEVKAPKETKEVKPATKADNNTYPILNQELREAIKNPAIKDKDHSAPNSRPIDFEMKKKDGTQQFYHYASSVKPARVIFTDSKPEIELGLQSGQFWRKFEVYEGNKKLPIKLVSYDTVKDYAYIRFSVSNGTKAVKIVSSTHFNNKEEKYDYTLMEFAQPIYNSADKFKTEEDYKAEKLLSPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKSNKKEQQDNSAKKETTPATPSKPTTPPVEKESQKQDSQKDDNKQSPSVEKENDASSESGKDKTPATKPAKAEVESSSTTPTKVVSTTQNVAKPTTASSETTIDVVQTSAGSSEAKDSAPLQKANIKNTNDGHTQSENNKNTQENKAKS

SEQ ID NO: 25 - protein, 568 aa, Artificial Sequence
  ORF0657nH
AEETGGTITETQPKTEAVASPTTTTEKAPEAKPVANAVSVSNKEVAAPTTETKEAKEVKEVKAPNETKEVKPAAKSDNNTYPILNEELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGERVRTISKDAKNNTRTIIFPYVEGKTLYDAIVKVHVKTIDYDGQYHVRIVDKEAFTKANADKSNKKEQQDNSAKKETTPATPSKPTTAPVEKESQKQDSQKDDNKQSPSVEKEIDASSESGKDKTPATKPAKGEVESSSTTPTKVVSATQNVAKPTSASSETTKGVVQTSAGSSEAKDNAPLQKANIKNTNDGHTQSQNNKNTQENKAKS

SEQ ID NO: 26 - protein, 568 aa, Artificial Sequence
  ORF0657nH
AEETGGTITETQPKTEAVASPTTTTEKAPEAKPVANAVSVSNKEVVAPTTETKEAKEVKEVKAPNETKEVKPAAKSDNNTYPILNEELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGERVRTISKDAKNNTRTIIFPYVEGKTLYDAIVKVHVKTIDYDGQYHVRIVDKEAFTKANADKSNKKEQQDNSAKKEATPATPSKPTTAPVEKESQKQDSQKDDNKQSPSVEKEIDASSESGKDKTPATKPAKGEVESSSTTPTKVVSATQNVAKPTSASSETTKGVVQTSAGSSEAKDNAPLQKANIKNTNDGHTQSQNNKNTQENKAKS

SEQ ID NO: 27 - protein, 570 aa, Artificial Sequence
  ORF0657nH
MGAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKGVTLATKPTKGEVESSSTTPTKVVSTTQNVAKPTTGSSKTTKDVVQTSAGSSEAKDSAPLQKANIKHTNDGHTQSQNNKNTQENKAKS

SEQ ID NO: 28 - protein, 654 aa, Artificial Sequence
  SEQ ID NO 2 modified to contain a glycine after the amino terminus methionine and a carboxyl His-Tag
MGNKQQKEFKSFYSIRKSSLGVASVAISTLLLLMSNGEAQAAAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKNLEHHHHHH

SEQ ID NO: 29 - DNA, 1962 bp, Artificial Sequence
  Full length ORF0657n + Carboxyl His-Tag
atgaacaaacagcaaaaagaatttaaatcattttattcaattagaaagtcatcactaggcgttgcatctgtagcaattagtacacttttattattaatgtcaaatggcgaagcacaagcagcagctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagaagttaaaccagcagcaaaagccactaacaatacatatcctattttgaatcaggaacttagagaagcgattaaaaaccctgcaataaaagacaaagatcatagcgcaccaaactctcgtccaattgattttgaaatgaaaaagaaagatggaactcaacagttttatcattatgcaagttctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattatgcttacattcgcttctctgtatcaaacggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcattaccacaaactggtgaagaatcaaataaagatatgacattaccattaatggcattattagctttaagtagcatcgttgcattcgtattacctagaaaacgtaaaaacctcgagcaccaccaccaccaccactga

SEQ ID NO: 30 - DNA, 1737 bp, Artificial Sequence
  ORF0657nH + Carboxyl His-Tag
atgaacgctgaagaaacaggtggtacaaatacagaagcacaaccaaaaactgaagcagttgcaagtccaacaacaacatctgaaaaagctccagaaactaaaccagtagctaatgctgtctcagtatctaataaagaagttgaggcccctacttctgaaacaaaagaagctaaagaagttaaagaagttaaagcccctaaggaaacaaaagaagttaaaccagcagcaaaagccactaacaatacatatcctattttgaatcaggaacttagagaagcgattaaaaaccctgcaataaaagacaaagatcatagcgcaccaaactctcgtccaattgattttgaaatgaaaaagaaagatggaactcaacagttttatcattatgcaagttctgttaaacctgctagagttattttcactgattcaaaaccagaaattgaattaggattacaatcaggtcaattttggagaaaatttgaagtttatgaaggtgacaaaaagttgccaattaaattagtatcatacgatactgttaaagattatgcttacattcgcttctctgtatcaaacggaacaaaagctgttaaaattgttagttcaacacacttcaataacaaagaagaaaaatacgattacacattaatggaattcgcacaaccaatttataacagtgcagataaattcaaaactgaagaagattataaagctgaaaaattattagcgccatataaaaaagcgaaaacactagaaagacaagtttatgaattaaataaaattcaagataaacttcctgaaaaattaaaggctgagtacaagaagaaattagaggatacaaagaaagctttagatgagcaagtgaaatcagctattactgaattccaaaatgtacaaccaacaaatgaaaaaatgactgatttacaagatacaaaatatgttgtttatgaaagtgttgagaataacgaatctatgatggatacttttgttaaacaccctattaaaacaggtatgcttaacggcaaaaaatatatggtcatggaaactactaatgacgattactggaaagatttcatggttgaaggtcaacgtgttagaactataagcaaagatgctaaaaataatactagaacaattattttcccatatgttgaaggtaaaactctatatgatgctatcgttaaagttcacgtaaaaacgattgattatgatggacaataccatgtcagaatcgttgataaagaagcatttacaaaagccaataccgataaatctaacaaaaaagaacaacaagataactcagctaagaaggaagctactccagctacgcctagcaaaccaacaccatcacctgttgaaaaagaatcacaaaaacaagacagccaaaaagatgacaataaacaattaccaagtgttgaaaaagaaaatgacgcatctagtgagtcaggtaaagacaaaacgcctgctacaaaaccaactaaaggtgaagtagaatcaagtagtacaactccaactaaggtagtatctacgactcaaaatgttgcaaaaccaacaactgcttcatcaaaaacaacaaaagatgttgttcaaacttcagcaggttctagcgaagcaaaagatagtgctccattacaaaaagcaaacattaaaaacacaaatgatggacacactcaaagccaaaacaataaaaatacacaagaaaataaagcaaaatcactcgagcaccaccaccaccaccactga

SEQ ID NO: 31 - DNA, 1941 bp, Artificial Sequence
  Encodes SEQ ID NO 28 without a carboxyl His-Tag and is codon optimized for yeast expression
atgggtaacaagcaacaaaaggaattcaagtctttctactccattagaaagtcttccttgggtgttgcttctgtcgctatctccaccttgttgttgttgatgtctaacggtgaagctcaagctgctgctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccaactaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtctttgccacaaaccggtgaagaatccaacaaggacatgaccttgccattgatggctttgttggctttgtcttccatcgttgctttcgtcttgccaagaaagagaaagaactaa

SEQ ID NO: 32 - DNA, 1710 bp, Artificial Sequence
  Encodes SEQ ID NO 3 and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccaactaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtcttaa

SEQ ID NO: 33 - DNA, 1341 bp, Artificial Sequence
  Encodes SEQ ID NO 1 and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccataa

SEQ ID NO: 34 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggtgtcactttggctaccaagccaactaagggtgaagttgaatcttcctctactactccaaccaaggttgtctccactacccaaaacgtcgctaagccaactaccggttcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 35 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggcgtcactttggctaccaagccaactaagggtgaagttgaatcttcctctactactccaaccaaggttgtctccactacccaaaacgtcgctaagccaactaccggttcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 36 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggtgttactttggctaccaagccaactaagggtgaagttgaatcttcctctactactccaaccaaggttgtctccactacccaaaacgtcgctaagccaactaccggttcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 37 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggcgttactttggctaccaagccaactaagggtgaagttgaatcttcctctactactccaaccaaggttgtctccactacccaaaacgtcgctaagccaactaccggttcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 38 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggtgtcactttagctaccaagccaactaagggtgaagttgaatcttcctctactactccaaccaaggttgtctccactacccaaaacgtcgctaagccaactaccggttcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 39 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggtgtcactttggctaccaagccaactaagggtgaagttgaatcttcctctactactccaaccaaggttgtctccactacccaaaacgtcgctaagccaactaccggctcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 40 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggtgttactttagctaccaagccaactaagggtgaagttgaatcttcctctactactccaaccaaggttgtctccactacccaaaacgtcgctaagccaactaccggttcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 41 - DNA, 1710 bp, Artificial Sequence
  Encodes SEQ ID NO 7 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagggtgttactttggctaccaagccaactaagggtgaagttgaatcttcctctactactccaaccaaggttgtctccactacccaaaacgtcgctaagccaactaccggctcttccaagactaccaaggacgttgtccaaacttctgctggttcctctgaagctaaggactctgctccattgcaaaaggctaacatcaagcacaccaacgacggtcacacccaatcccaaaacaacaagaacactcaagaaaacaaggctaagtcttaa

SEQ ID NO: 42 - protein, 481 aa, Artificial Sequence
  ORF0657nI+
MAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGK

SEQ ID NO: 43 - DNA, 1452 bp, Artificial Sequence
  Encodes SEQ ID NO 42 and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagtaaggatcc

SEQ ID NO: 44 - protein, 605 aa, Artificial Sequence
  ORF0657nG
MAEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 45 - DNA, 1818 bp, Artificial Sequence
  Encodes SEQ ID NO 44 containing an amino terminus methionine and is codon optimized for yeast expression
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggaagtcaagccagctgctaaggctaccaacaacacttacccaattttgaaccaagaattgagagaagctattaagaacccagctatcaaggacaaggaccactccgctccaaactctagaccaatcgacttcgaaatgaagaagaaggacggtacccaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccaactaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtctttgccacaaaccggtgaagaatccaacaaggacatgaccttgccattgatggctttgttggctttgtcttccatcgttgctttcgtcttgccaagaaagagaaagaactaa

SEQ ID NO: 46 - DNA, 1710 bp, Artificial Sequence
  Encodes SEQ ID NO 17 containing an amino terminus methionine and is codon optimized for yeast expression
atggctgaagaaactggtggtaccaacactgaagctcaaccaaagaccgaagctttggcttccccaaccactaccactgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctgctaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccagctaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtcttaa

SEQ ID NO: 47 - DNA, 1446 bp, Artificial Sequence
  Encodes the SEQ ID NO 17 I+ region, is codon optimized for yeast expression, and encodes a methionine initiation codon
atggctgaagaaactggtggtaccaacactgaagctcaaccaaagaccgaagctttggcttccccaaccactaccactgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctgctaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagtaa

SEQ ID NO: 48 - DNA, 1341 bp, Artificial Sequence
  Encodes the SEQ ID NO 17 I region, is codon optimized for yeast expression, and encodes a methionine initiation codon
atggctgaagaaactggtggtaccaacactgaagctcaaccaaagaccgaagctttggcttccccaaccactaccactgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctgctaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccataa

SEQ ID NO: 49 - DNA, 1938 bp, Artificial Sequence
  Encodes for full length ORF0657n containing SEQ ID NO 17 modified to contain a glycine afer the amino terminus methionine and is codon optimized for yeast expression
atgggtaacaagcaacaaaaggaattcaagtctttctactccattagaaagtcttccttgggtgttgcttctgtcgctatctccaccttgttgttgttgatgtctaacggtgaagctcaagctgctgaagaaactggtggtaccaacactgaagctcaaccaaagaccgaagctttggcttccccaaccactaccactgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctgctaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccagctaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtctttgccacaaaccggtgaagaatccaacaaggacatgaccttgccattgatggctttgttggctttgtcttccatcgttgctttcgtcttgccaagaaagagaaagaactaa

SEQ ID NO: 50 - DNA, 1710 bp, Artificial Sequence
  Encodes SEQ ID NO 20, is codon optimized for yeast expression, and encodes a methionine initiation codon
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctactaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccagctaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtcttaa

SEQ ID NO: 51 - DNA, 1446 bp, Artificial Sequence
  Encodes SEQ ID NO 20 I+ region, is codon optimized for yeast expression, and encodes a methionine initiation codon
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctactaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaagtaa

SEQ ID NO: 52 - DNA, 1341 bp, Artificial Sequence
  Encodes the SEQ ID NO 20 I region, is codon optimized for yeast expression, and encodes a methionine initiation codon
atggctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctactaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccataa

SEQ ID NO: 53 - DNA, 1938 bp, Artificial Sequence
  Encodes for full length ORF0657n containing SEQ ID NO 20 modified to contain a glycine after the amino terminus methionine and is codon optimized for yeast expression
atgggtaacaagcaacaaaaggaattcaagtctttctactccattagaaagtcttccttgggtgttgcttctgtcgctatctccaccttgttgttgttgatgtctaacggtgaagctcaagctgctgaagaaactggtggtaccaacactgaagctcaaccaaagaccgaagctgtcgcttccccaaccactacctctgaaaaggctccagaaactaagccagttgctaacgctgtctccgtttctaacaaggaagtcgaagctccaacctccgaaactaaggaagctaaggaagttaaggaagtcaaggctccaaaggaaactaaggctgtcaagccagctactaaggctgacaacaacacttacccaattttgaaccaagaattgagagaagctattaagaacccagctatcaaggacaaggaccactccgctccaaactctagaccaatcgacttcgaaatgaagaaggaaaacggtgaacaacaattctaccactacgcgtcctctgtcaagccagctagagttattttcaccgactctaagccagaaatcgaattgggtttgcaatccggtcaattctggagaaagttcgaagtctacgaaggtgacaagaagttgccaattaagttggtttcctacgacaccgtcaaggactacgcttacatcagattctccgtttctaacggtactaaggctgtcaagattgtctcttccacccacttcaacaacaaggaagaaaagtacgactacactttgatggaattcgctcaaccaatttacaactctgctgacaagttcaagaccgaagaagactacaaggctgaaaagttgttggctccatacaagaaggctaagactttggaaagacaagtttacgaattgaacaagatccaagacaagttgccagaaaagttgaaggctgaatacaagaagaagttggaagacaccaagaaggctttggacgaacaagtcaagtccgctatcaccgaattccaaaacgttcaaccaactaacgaaaagatgactgacttgcaagacactaagtacgtcgtctacgaatccgtcgaaaacaacgaatccatgatggacaccttcgttaagcacccaattaagactggtatgttgaacggtaagaagtacatggtcatggaaaccactaacgacgactactggaaggacttcatggttgaaggtcaaagagtcagaaccatctccaaggacgctaagaacaacactagaaccattatcttcccatacgttgaaggtaagactttgtacgacgctatcgtcaaggttcacgtcaagactattgactacgacggtcaataccacgttagaattgttgacaaggaagctttcaccaaggctaacaccgacaagtccaacaagaaggaacaacaagacaactctgctaagaaggaagctaccccagctaccccatctaagccaaccccatctccagttgaaaaggaatctcaaaagcaagactcccaaaaggacgacaacaagcaattgccatccgtcgaaaaggaaaacgacgcgtcttctgaatccggtaaggacaagactccagctaccaagccagctaagggtgaagttgaatcttcctctactactccaaccaaggttgtctccactacccaaaacgtcgctaagccaactaccgcttcttccaagactaccaaggacgttgtccaaacttctgctggttcctctgaagctaaggactctgctccattgcaaaaggctaacatcaagaacaccaacgacggtcacacccaatcccaaaacaacaagaacactcaagaaaacaaggctaagtctttgccacaaaccggtgaagaatccaacaaggacatgaccttgccattgatggctttgttggctttgtcttccatcgttgctttcgtcttgccaagaaagagaaagaactaa

SEQ ID NO: 54 - protein, 565 aa, Artificial Sequence
  ORF0657nH
AEETGGTNTEAQPKTEAVASPTTTTEKAPEAKPVANAVSVSNKEVEAPTSETKEAKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYAVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSASSSEAKDSAPLQKANIKNTNDGHTQSQNNKNTQENKAKS

SEQ ID NO: 55 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQGLREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKPAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPITASSKTTKDVVQTSAGSSEAKDSAPLQKANIKNTNDGHTQSQNNKNTQENKAKS

SEQ ID NO: 56 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPEIKDKDHSAPNSRPIDFEMKKKDGTQQFYHYASSVKPARVIFTDSKPEIELGLQSGQFWRKFEVYEGDKKLPIKLVSYDTVKDYAYIRFSISNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVVKPTTASSKTTKDVVQTSAGSSEAKDSAPLQKANIKNTNDGHTQSQNNKNTQENKAKS

SEQ ID NO: 57 - protein, 568 aa, Artificial Sequence
  ORF0657nH
AEETGGTNTEAQPKTEAVASPTTTSEKAPETKPVANAVPVSNKEVEAPTSETKEAKEVKEVKAPKETKEVKPAAKATNNTYPILNQELREATKNPEIKDKDHSAPNSRPTDFEMKKNDGTQQFYHYASSVKPARVIFTDSKPEIELGLQSGQFWRKFEVYEGDKKLPIKLVSYDTVKDYAYIRFSISNGTKAVKIVSSTHFNNKEEKYDYTLMEFAQPIYNSADKFKTEEDYKAEKLLAPYKKAKTXERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANLLKTHDGHTQSQNIKNTKKDKAKS

SEQ ID NO: 58 - protein, 568 aa, Artificial Sequence
  ORF0657nH
AEETGGTNTEAQPKTEAVASPTTTSEKAPETKPVANAVSVSNKEVEAPTSETKEAKEVKEVKAPKETKEVKPAAKATNNTYPILNQELREAIKNPAIKDKDHSAPNSRPIDFEMKKENGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 59 - protein, 567 aa, Artificial Sequence
  ORF0657nH
AEETGGTNTEAQPKTEAVASPTTTSEKAPETKPVANAVSVSNKEVEAPTSETKEAKEVKEVKAPKETKAVKPAAKATNNTYPILNQELREAIKNPAIKDKDHSAPNSRPIDFEMNKKNGEQQFYHYASSAKPARVIFTDSKPEIELGLQSGQFWRKFEVYEGDKKLPIKLVSYDTVKDYAYIRFSVSNGTKAVKIVSSTHFNNKEEKDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPTKGEVESSSTTPTKVVSTTQNVAKPTTASSKTTKDVVQTSAGSSEAKDSAPLQKANIKNTNDGHTQSQNNKNTQENKAKS

SEQ ID NO: 60 - protein, 576 aa, Artificial Sequence
  ORF0657nH
AEETGVTNTEAQPKTEAVASPTTTTTEKAPEAKPVAKPVANAVSVSNKEVVAPTTETKEAKEVKAVKEVKAPKEAKEEKPAAKADNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVAYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKTNKKEQQDNSAKKETTPAMPSKPTTPPVEKESQKQDSQKDDNKQSPSVEKENDASSESGKDKMPVTKPAKAEVESSSTTPTKVVSTTQNVAKPTTASSETTKDVVQTSAGSSEAKDSAPLQKANIKNTNDGHTQSQNNKNTQENKAKS

SEQ ID NO: 61 - protein, 572 aa, Artificial Sequence
  ORF0657nH
AEETGVTNTEAQPKTEAVASPTTTTTEKAPEAKPVANAVSVSNKEVVAPTTETKEAKEVKAVKEVKAPKEAKEEKPAAKADNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPCVEGKTLYDAIVKVHVKTIDYDGQYHVRIVDKEAFTKANADKTNKKEQQDNSAKKETTPAMPSKPTTPPVEKESQKQDSQKDDNKQSPSVEKENDASSESGKDKMPVTKPAKAEVESSSTTPTKVVSTTQNVAKPTTASSETTKDVVQTSAGSSEAKDSAPLQKANIKNTNDGHTQSQNNKNTQENKAKS

SEQ ID NO: 62 - protein, 572 aa, Artificial Sequence
  ORF0657nH
AEETGVTNTEAQPKTEAVASPTTTTTEKAPEAKPVANAVSVSNKEVVAPTTETKEAKEVKAVKEVKAPKEAKEEKPAAKADNNTYPILNQELREAIKNPAIKDKDHSAPNSRPIDFEMKKKDGTQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANADKTNKKEQQDNSAKKETTPAMPSKPTTPPVEKESQKQDSQKDDNKQSPSVEKENDASSESGKDKMPVTKPAKAEVESSSTTPTKVVSTTQNVAKPTTASSETTKDVVQTSAGSSEAKDSAPLQKANIKNTNDGHTQSQNNKNTQENKAKS

SEQ ID NO: 63 - protein, 566 aa, Artificial Sequence
  ORF0657nH
AEETGGTNTEAQPKTEAVASPTTTTTEKAPEAKPVANAVSVSNKEVVAPTTETKEAKEVKAPKETKEVKPAAKADNNTYPILNKELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEETKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYIEGKTLYDAIVKVHVKTIDYDGQYHVRIVDKEAFTKANADKSNKKEQQDNSAKKETTPATPSKPTTPPVEKESQKQDSQKDDNTQSPSVEKENDASSESGKDKTPATKPAKGEVESSSTTPTKVVSTTQNVAKPTTASSETTKDVVQTSAGPSEAKDSAPLQKANIKNTNDGHTQSQNNKNTQEDKAKS

SEQ ID NO: 64 - protein, 8 aa, Artificial Sequence
  His-Tag
LEHHHHHH

SEQ ID NO: 65 - DNA, 16 bp, Artificial Sequence
  Primer
ctggccgtcgttttac

SEQ ID NO: 66 - DNA, 17 bp, Artificial Sequence
  Primer
caggaaacagctatgac

SEQ ID NO: 67 - DNA, 39 bp, Artificial Sequence
  Primer
aaccggttttccatggggaacaaacagcaaaaagaattt

SEQ ID NO: 68 - DNA, 38 bp, Artificial Sequence
  Primer
accggtttctcgaggtttttacgttttctaggtaatac

SEQ ID NO: 69 - DNA, 109 bp, Artificial Sequence
  ORF0657n oligomer
atgggtaacaagcaacaaaaggaattcaagtctttctactccattagaaagtcttccttgggtgttgcttctgtcgctatctccaccttgttgttgttgatgtctaacg

SEQ ID NO: 70 - DNA, 110 bp, Artificial Sequence
  ORF0657n oligomer
gttggggaagcgacagcttcggtctttggttgagcttcagtgttggtaccaccagtttcttcagcagcagcttgagcttcaccgttagacatcaacaacaacaaggtgga

SEQ ID NO: 71 - DNA, 110 bp, Artificial Sequence
  ORF0657n oligomer
agaccgaagctgtcgcttccccaaccactacctctgaaaaggctccagaaactaagccagttgctaacgctgtctccgtttctaacaaggaagtcgaagctccaacctcc

SEQ ID NO: 72 - DNA, 109 bp, Artificial Sequence
  ORF0657n oligomer
tggtagccttagcagctggcttgacttccttagtttcctttggagccttgacttccttaacttccttagcttccttagtttcggaggttggagcttcgacttccttgtt

SEQ ID NO: 73 - DNA, 108 bp, Artificial Sequence
  ORF0657n oligomer
aagtcaagccagctgctaaggctaccaacaacacttacccaattttgaaccaagaattgagagaagctattaagaacccagctatcaaggacaaggaccactccgctc

SEQ ID NO: 74 - DNA, 109 bp, Artificial Sequence
  ORF0657n oligomer
tggcttgacagaggacgcgtagtggtagaattgttgggtaccgtccttcttcttcatttcgaagtcgattggtctagagtttggagcggagtggtccttgtccttgata

SEQ ID NO: 75 - DNA, 102 bp, Artificial Sequence
  ORF0657n oligomer
accactacgcgtcctctgtcaagccagctagagttattttcaccgactctaagccagaaatcgaattgggtttgcaatccggtcaattctggagaaagttcg

SEQ ID NO: 76 - DNA, 104 bp, Artificial Sequence
  ORF0657n oligomer
ctgatgtaagcgtagtccttgacggtgtcgtaggaaaccaacttaattggcaacttcttgtcaccttcgtagacttcgaactttctccagaattgaccggattg

SEQ ID NO: 77 - DNA, 109 bp, Artificial Sequence
  ORF0657n oligomer
caccgtcaaggactacgcttacatcagattctccgtttctaacggtactaaggctgtcaagattgtctcttccacccacttcaacaacaaggaagaaaagtacgactac

SEQ ID NO: 78 - DNA, 109 bp, Artificial Sequence
  ORF0657n oligomer
aacttttcagccttgtagtcttcttcggtcttgaacttgtcagcagagttgtaaattggttgagcgaattccatcaaagtgtagtcgtacttttcttccttgttgttga

SEQ ID NO: 79 - DNA, 106 bp, Artificial Sequence
  ORF0657n oligomer
ccgaagaagactacaaggctgaaaagttgttggctccatacaagaaggctaagactttggaaagacaagtttacgaattgaacaagatccaagacaagttgccaga

SEQ ID NO: 80 - DNA, 109 bp, Artificial Sequence
  ORF0657n oligomer
tcggtgatagcggacttgacttgttcgtccaaagccttcttggtgtcttccaacttcttcttgtattcagccttcaacttttctggcaacttgtcttggatcttgttca

SEQ ID NO: 81 - DNA, 109 bp, Artificial Sequence
  ORF0657n oligomer
cgaacaagtcaagtccgctatcaccgaattccaaaacgttcaaccaactaacgaaaagatgactgacttgcaagacactaagtacgtcgtctacgaatccgtcgaaaac

SEQ ID NO: 82 - DNA, 109 bp, Artificial Sequence
  ORF0657n oligomer
tttccatgaccatgtacttcttaccgttcaacataccagtcttaattgggtgcttaacgaaggtgtccatcatggattcgttgttttcgacggattcgtagacgacgta

SEQ ID NO: 83 - DNA, 109 bp, Artificial Sequence
  ORF0657n oligomer
gaacggtaagaagtacatggtcatggaaaccactaacgacgactactggaaggacttcatggttgaaggtcaaagagtcagaaccatctccaaggacgctaagaacaac

SEQ ID NO: 84 - DNA, 101 bp, Artificial Sequence
  ORF0657n oligomer
gtcttgacgtgaaccttgacgatagcgtcgtacaaagtcttaccttcaacgtatgggaagataatggttctagtgttgttcttagcgtccttggagatggt

SEQ ID NO: 85 - DNA, 106 bp, Artificial Sequence
  ORF0657n oligomer
cgctatcgtcaaggttcacgtcaagactattgactacgacggtcaataccacgttagaattgttgacaaggaagctttcaccaaggctaacaccgacaagtccaac

SEQ ID NO: 86 - DNA, 96 bp, Artificial Sequence
  ORF0657n oligomer
tggggttggcttagatggggtagctggggtagcttccttcttagcagagttgtcttgttgttccttcttgttggacttgtcggtgttagccttggt

SEQ ID NO: 87 - DNA, 85 bp, Artificial Sequence
  ORF0657n oligomer
cagctaccccatctaagccaaccccatctccagttgaaaaggaatctcaaaagcaagactcccaaaaggacgacaacaagcaatt

SEQ ID NO: 88 - DNA, 100 bp, Artificial Sequence
  ORF0657n oligomer
gttggcttggtagctggagtcttgtccttaccggattcagaagacgcgtcgttttccttttcgacggatggcaattgcttgttgtcgtccttttgggagt

SEQ ID NO: 89 - DNA, 101 bp, Artificial Sequence
  ORF0657n oligomer
ggacaagactccagctaccaagccaactaagggtgaagttgaatcttcctctactactccaaccaaggttgtctccactacccaaaacgtcgctaagccaa

SEQ ID NO: 90 - DNA, 101 bp, Artificial Sequence
  ORF0657n oligomer
agcagagtccttagcttcagaggaaccagcagaagtttggacaacgtccttggtagtcttggaagaagcggtagttggcttagcgacgttttgggtagtgg

SEQ ID NO: 91 - DNA, 91 bp, Artificial Sequence
  ORF0657n oligomer
ggttcctctgaagctaaggactctgctccattgcaaaaggctaacatcaagaacaccaacgacggtcacacccaatcccaaaacaacaaga

SEQ ID NO: 92 - DNA, 98 bp, Artificial Sequence
  ORF0657n oligomer
gtgaagaatccaacaaggacatgaccttgccattgatggctttgttggctttgtcttccatcgttgctttcgtcttgccaagaaagagaaagaactaa

SEQ ID NO: 93 - DNA, 98 bp, Artificial Sequence; identical to SEQ ID NO: 92
  ORF0657n oligomer

SEQ ID NO: 94 - DNA, 31 bp, Artificial Sequence
  Primer
cttaaagcttatgtcactttctcttgtatcg

SEQ ID NO: 95 - DNA, 30 bp, Artificial Sequence
  Primer
tgataagcttgctcaatggttctcttcctc

SEQ ID NO: 96 - DNA, 53 bp, Artificial Sequence
  Primer
aaccggtttggatcccacaaaacaaaatgggtaacaagcaacaaaaggaattc

SEQ ID NO: 97 - DNA, 42 bp, Artificial Sequence
  Primer
aaccggtttggatccttagttctttctctttcttggcaagac

SEQ ID NO: 98 - DNA, 24 bp, Artificial Sequence
  Primer
gctgaagaaactggtggtaccaac

SEQ ID NO: 99 - DNA, 42 bp, Artificial Sequence
  Primer
gtcacggatccttaagacttagccttgttttcttgagtgttc

SEQ ID NO: 100 - DNA, 41 bp, Artificial Sequence
  Primer
ggggggatcccacaaaacaaaatggctgaagaaactggtgg

SEQ ID NO: 101 - DNA, 39 bp, Artificial Sequence
  Primer
ggggggggatccttaagacttagccttgttttcttgagt

SEQ ID NO: 102 - DNA, 41 bp, Artificial Sequence; identical to SEQ ID NO: 100
  Primer

SEQ ID NO: 103 - DNA, 32 bp, Artificial Sequence
  Primer
gggggggatccttagttctttctctttcttgg

SEQ ID NO: 104 - DNA, 39 bp, Artificial Sequence
  Primer
ctccggatcccacaaaacaaaatggctgaagaaactggt

SEQ ID NO: 105 - DNA, 38 bp, Artificial Sequence
  Primer
gctgccgggatccttatggggttggcttagatggggta

SEQ ID NO: 106 - protein, 644 aa, S. aureus
MNKQQKEFKSFYSIRKSSLGVASVAISTLLLLMSNGEAQAAEETGGTNTEAQPKTEALASPTTTTEKAPETKPVANAVSVSNKEVEAPTSETKEAKEVKEVKAPKETKAVKPAAKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAINNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 107 - protein, 644 aa, S. aureus
MNKQQKEFKSFYSIRKSSLGVASVAISTLLLLMSNGEAQAAEETGGTNTEAQPKTEAVASPTTTSEKAPETKPVANAVSVSNKEVEAPTSETKEAKEVKEVKAPKETKAVKPATKADNNTYPILNQELREAIKNPAIKDKDHSAPNSRPIDFEMKKENGEQQFYHYASSVKPARVIFTDSKPEIELGLQSGQFWRKFEVYEGDKKLPIKLVSYDTVKDYAYIRFSVSNGTKAVKIVSSTHFNNKEEKYDYTLMEFAQPIYNSADKFKTEEDYKAEKLLAPYKKAKTLERQVYELNKIQDKLPEKLKAEYKKKLEDTKKALDEQVKSAITEFQNVQPTNEKMTDLQDTKYVVYESVENNESMMDTFVKHPIKTGMLNGKKYMVMETTNDDYWKDFMVEGQRVRTISKDAKNNTRTIIFPYVEGKTLYDAIVKVHVKTIDYDGQYHVRIVDKEAFTKANTDKSNKKEQQDNSAKKEATPATPSKPTPSPVEKESQKQDSQKDDNKQLPSVEKENDASSESGKDKTPATKPAKGEVESSSTTPTKVVSTTQNVAKPTTASSKTTKDVVQTSAGSSEAKDSAPLQKANIKNTNDGHTQSQNNKNTQENKAKSLPQTGEESNKDMTLPLMALLALSSIVAFVLPRKRKN

SEQ ID NO: 108 - protein, 5 aa, Artificial Sequence
  cell wall sorting signal
LPXTG

SEQ ID NO: 109 - protein, 7 aa, Artificial Sequence
  cell wall sorting signal
LPXTGVI

(19) United States
(12) Patent Application Publication
(10) Pub. No.: US 2010/0247561 A1
(43) Pub. Date: Sep. 30, 2010

(54) POLYPEPTIDES FOR INDUCING A PROTECTIVE IMMUNE RESPONSE AGAINST STAPHYLOCOCCUS AUREUS

(76) Inventors: Annaliesa S. Anderson, Doylestown, PA (US); Kathrin Ute Jansen, South San Francisco, CA (US); Rosemarie Kelly, Westfield, NJ (US); Loren D. Schultz, Harleysville, PA (US); Donna L. Montgomery, Chalfont, PA (US); William L. McClements, Doylestown, PA (US)

Correspondence Address: MERCK, P O BOX 2000, RAHWAY, NJ, 07065-0907, US

(21) Appl. No.: 12/771,345

(22) Filed: Apr. 30, 2010

Related U.S. Application Data
(62) Division of application No. 10/564,458, filed on Jan. 12, 2006.
(60) Provisional application No. 60/489,840, filed on Jul. 24, 2003.
(60) Provisional application No. 60/520,115, filed on Nov. 14, 2003.

(51) Int. Cl. A61K 39/085; A61K 39/00; A61P 37/04; A61P 31/04

(52) U.S. Cl. 424/190.1; 424/185.1

(57) ABSTRACT

The present invention features polypeptides comprising an amino acid sequence structurally related to SEQ ID NO: 1, uses of such polypeptides, and expression systems for producing such polypeptides. SEQ ID NO: 1 is a truncated derivative of a full length S. aureus polypeptide. The full-length polypeptide is referred to herein as full-length “ORF0657n”. Polypeptides containing the amino acid sequence of SEQ ID NO: 1 were found to produce a protective immune response against S. aureus.

### CROSS-REFERENCE TO RELATED APPLICATIONS

The present application claims the benefit of U.S. Provisional Application No. 60/489,840, filed Jul. 24, 2003 and U.S. Provisional Application No. 60/520,115, filed Nov. 14, 2003, each of which is hereby incorporated by reference herein.

### BACKGROUND OF THE INVENTION

The references cited throughout the present application are not admitted to be prior art to the claimed invention.

Staphylococcus aureus is a pathogen responsible for a wide range of diseases and conditions. Examples of diseases and conditions caused by S. aureus include bacteremia, infective endocarditis, folliculitis, furuncle, carbuncle, impetigo, bullous impetigo, cellulitis, botryomyosis, toxic shock syndrome, scalded skin syndrome, central nervous system infections, infective and inflammatory eye disease, osteomyletitis and other infections of joints and bones, and respiratory tract infections. (The Staphylococci in Human Disease, Crossley and Archer (eds.), Churchill Livingstone Inc. 1997.)

Immunological based strategies can be employed to try to control S. aureus infections and the spread of S. aureus. Immunological based strategies include passive and active immunization. Passive immunization employs immunoglobulins targeting S. aureus. Active immunization induces immune responses against S. aureus.

Potential S. aureus vaccines target S. aureus polysaccharides and polypeptides. Targeting can be achieved using suitable S. aureus polysaccharides or polypeptides as vaccine components. Examples of potential polysaccharides vaccine components include S. aureus type 5 and type 8 capsular polysaccharides. (Shinefield et al., N. Eng. J. Med. 346:491-496, 2002.) Examples of polypeptides that may be employed as possible vaccine components include collagen adhesin, fibrinogen binding proteins, and clumping factor. (Mamo et al., FEMS Immunology and Medical Microbiology 10:47-54, 1994, Nilsson et al., J. Clin. Invest. 101:2640-2649, 1998, Josefsson et al., The Journal of Infectious Diseases 184:1572-1580, 2001.)

Information concerning S. aureus polypeptide sequences has been obtained from sequencing the S. aureus genome. (Kuroda et al., Lancet 357:1225-1240, 2001, Baba et al., Lancet 359:1819-1827, 2000, Kunsch et al., European Patent Publication EP 0 786 519, published Jul. 30, 1997.) To some extent bioinformatics has been employed in efforts to characterize polypeptide sequences obtained from genome sequencing. (Kunsch et al., European Patent Publication EP 0 786 519, published Jul. 30, 1997.)

Techniques such as those involving display technology and sera from infected patients can be used as part of an effort to try to identify genes coding for potential antigens. (Foster et al., International Publication Number WO 01/98499, published Dec. 27, 2001, Meinke et al., International Publication Number WO 02/059148, published Aug. 1, 2002.)

### SUMMARY OF THE INVENTION

The present invention features polypeptides comprising an amino acid sequence structurally related to SEQ ID NO: 1, uses of such polypeptides, and expression systems for producing such polypeptides. SEQ ID NO: 1 is a truncated derivative of a full length S. aureus polypeptide. The full-length polypeptide is referred to herein as full-length “ORF0657n”. Polypeptides containing the amino acid sequence of SEQ ID NO: 1 were found to produce a protective immune response against S. aureus.

Reference to “protective” immunity or immune response indicates a detectable level of protection against S. aureus infection. The level of protection can be assessed using animal models such as those described herein.

Thus, a first aspect of the present invention describes a polypeptide immunogen comprising an amino acid sequence at least 90% identical to SEQ ID NO: 1, wherein the polypeptide does not contain a carboxyl terminus provided by amino acids 609-645 of SEQ ID NO: 2 and the polypeptide provides protective immunity against S. aureus. SEQ ID NO: 2 provides a full length ORF0657n polypeptide, wherein amino acids 609-645 provide the carboxyl terminus domain starting at the LPXTG motif (as referred to herein as the “cell well sorting signal”).

Reference to “immunogen” indicates the ability to provide protective immunity.

Reference to comprising an amino acid sequence at least 90% identical to SEQ ID NO: 1 indicates that a SEQ ID NO: 1 related region is present and additional polypeptide regions may be present. If additional polypeptide regions are present, then the polypeptide does not have a carboxyl LPXTG motif as provided by amino acids 609-645 of SEQ ID NO: 2.

Another aspect of the present invention describes an immunogen comprising an amino acid sequence that provides protective immunity against S. aureus. The immunogen comprises an amino acid sequence at least 90% identical to SEQ ID NO: 1 and one or more additional regions or moieties covalently joined at the carboxyl terminus or amino terminus, wherein each region or moiety is independently selected from a region or moiety having at least one of the following properties: enhances the immune response, facilitates purification, or facilitates polypeptide stability.

Reference to “additional region or moiety” indicates a region or moiety different from a ORF0657n related polypeptide which would be produced in a biological host, such as a prokaryotic or eukaryotic host. The additional region or moiety can be, for example, an additional polypeptide region or a non-peptide region.

Another aspect of the present invention describes a composition able to induce protective immunity against S. aureus in a patient. The composition comprises a pharmaceutically acceptable carrier and an immunologically effective amount of an immunogen providing protective immunity against S. aureus.

An immunologically effective amount is an amount sufficient to provide protective immunity against S. aureus infection. The amount should be sufficient to significantly prevent the likelihood or severity of a S. aureus infection.

Another aspect of the present invention describes a nucleic acid comprising a recombinant gene encoding a polypeptide that provides protective immunity against S. aureus. A recombinant gene contains recombinant nucleic acid encoding a polypeptide along with regulatory elements for proper transcription and processing (which may include translational and post translational elements). The recombinant gene can exist independent of a host genome or can be part of a host genome.

A recombinant nucleic acid is nucleic acid that by virtue of its sequence and/or form does not occur in nature. Examples of recombinant nucleic acid include purified nucleic acid, two or more nucleic acid regions combined together that provides a different nucleic acid than found in nature, and the absence of one or more nucleic acid regions (e.g., upstream or downstream regions) that are naturally associated with each other.

Another aspect of the present invention describes a recombinant cell. The cell comprises a recombinant gene encoding a polypeptide that provides protective immunity against S. aureus.

Another aspect of the present invention describes a method of making a polypeptide that provides protective immunity against S. aureus. The method involves growing a recombinant cell containing recombinant nucleic acid encoding the polypeptide and purifying the polypeptide.

Another aspect of the present invention describes a polypeptide that provides protective immunity against S. aureus made by a process comprising the steps of growing a recombinant cell containing recombinant nucleic acid encoding the polypeptide in a host and purifying the polypeptide. Different host cells can be employed. In an embodiment of the present invention the host cell is a yeast cell.

Another aspect of the present invention describes a method of inducing a protective immune response in a patient against S. aureus. The method comprises the step of administering to the patient an immunologically effective amount of an immunogen that provides protective immunity against S. aureus.

Another aspect of the present invention describes a method of inducing an anamnestic response in a patient. The method comprises the step of administering to the patient an effective amount of an immunogen to produce the anamnestic response.

Another aspect of the present invention describes nucleic acid encoding an ORF0657n related polypeptide that is optimized for expression in yeast. One or more codons are optimized for yeast expression.

Another aspect of the present invention describes a method of making a polypeptide that provides protective immunity against S. aureus in a recombinant yeast cell. The method comprises the steps of:

(a) growing a recombinant yeast cell under conditions wherein the polypeptide is expressed, wherein the recombinant yeast cell comprises a recombinant gene encoding the polypeptide and the polypeptide is a full-length ORF0657n related polypeptide that provides protective immunity against S. aureus infection, or a fragment thereof comprising an amino acid sequence at least 90% identical to SEQ ID NO: 1; and

(b) purifying the polypeptide.

Unless particular terms are mutually exclusive, reference to “or” indicates either or both possibilities. Occasionally phrases such as “and/or” are used to highlight either or both possibilities.

Reference to open-ended terms such as “comprises” allows for additional elements or steps. Occasionally phrases such as “one or more” are used with or without open-ended terms to highlight the possibility of additional elements or steps.

Unless explicitly stated reference to terms such as “a” or “an” is not limited to one. For example, “a cell” does not exclude “cells”. Occasionally phrases such as one or more are used to highlight the presence of a plurality.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIGS. 1A, 1B, 1C, and 1D provide a schematic illustrating ORF0657n related polypeptide regions screened for protection in animals, and some different ORF0657n sequences. FIG. 1A illustrates a schematic of polypeptides that were tested and found protective (shown by filled in rectangles), polypeptides tested and found not to be protective (shown by open rectangles), and a polypeptide not tested (hatched box). FIG. 1B provides a full-length sequence used as a reference for FIG. 1A (SEQ ID NO: 2). FIG. 1C illustrates SEQ ID NO: 28. SEQ ID NO: 28 contains a carboxyl “His-Tag” (LEHHHHHH; SEQ ID NO: 64). SEQ ID NO: 28 containing a carboxyl His-Tag is also referred to herein as “His-Tag ORF0657n”. FIG. 1D illustrates an ORF0657nI⁺ sequence.

FIGS. 2A-2E provides a sequence comparison of different ORF0657n related sequences across the ORF0657nH region. SEQ ID NOs: are indicated in the Figure by “ID”.

FIGS. 3A, 3B, and 3C illustrates the ability of ORF0657n related polypeptides providing the full-length sequence, the OFR0657nH region and the ORF0657nI region to provide protective immunity against S. aureus Becker. The polypeptides were used with an aluminum hydroxyphosphate adjuvant (AHP). FIG. 3A illustrates results with SEQ ID NO: 28. FIG. 3B illustrates results with SEQ ID NO: 4 containing a carboxyl His-Tag. FIG. 3C illustrates results with SEQ ID NO: 5 containing a carboxyl His-Tag. Reference to a “carboxyl His-Tag” indicates the His-Tag group LEHHHHHH (SEQ ID NO: 64) is present at the carboxyl terminus.

FIGS. 4A-4H illustrates the ability of ORF0657n related polypeptides to provide protective immunity against different S. aureus challenges. The polypeptide of SEQ ID NO: 28 was used as immunogen FIG. 4A shows results using challenge strain CL-10 (2.2×10⁸ CFU/ml). FIG. 4B shows results using challenge strain CL-10 (2.1×10⁸ CFU/ml). FIG. 4C shows results using challenge strain CL-13 (2.9×10⁸ CFU/ml). FIG. 4D shows results using challenge strain CL-13 (2.8×10⁸ CFU/ml). FIG. 4E shows results using challenge strain CL-30 (3.1×10⁸ CFU/ml). FIG. 4F shows results using challenge strain CL-30 (3.0×10⁸ CFU/ml), FIG. 4G shows results using challenge strain CL-18 (1.0×10⁸ CFU/ml. FIG. 4H shows results using challenge strain CL-21 (1.6×10⁸ CFU/ml).

FIGS. 5A and 5B illustrate plasmid maps of S. cerevisiae expression plasmids. FIG. 5A provides a plasmid map of vector pGAL110. FIG. 5B provides a plasmid map for piUC-I showing a yeast codon-optimized sequence cloned under control of the GAL1 promoter of pGAL110.

FIGS. 6A and 6B show Western blots depicting intracellular expression of a full-length ORF0657n having amino acids 1-646 of SEQ ID NO: 28 (SEQ ID NO: 28 without the carboxyl His-Tag). Lane 1, molecular size standards; lane 2, purified E. coli produced recombinant full-length ORF0657n region (SEQ ID NO: 28), 100 ng; lanes 3-6 contain 20 μg of yeast cell lysate; lanes 3 and 4, cell lysates from duplicate fermentations of transformants of 1260 (FIG. 6A) and 1309 (FIG. 6B) containing only vector pGAL110; lanes 5 and 6, cell lysates from duplicate fermentations of transformants of 1260 (FIG. 6A) and 1309 (FIG. 68) containing pRUnkC-pGAL110 which expresses full-length ORF0657n region (SEQ ID NO: 28 without the carboxyl His-Tag).

FIGS. 7A and 7B show a Coomassie stain of an SDS-PAGE gel and a Western blot, respectively, depicting intracellular expression in S. cerevisiae from nucleic acid encoding SEQ ID NO: 3. Lane 1, Panel A, BSA, 1.25 μg; Panel B, purified E. coli recombinant full length ORF0657n (SEQ ID NO: 28), 100 ng; lane 2, cell-lysate from producer of ORF0657nH (SEQ ID NO: 4 with a carboxyl His-Tag) in E. coli; Panel A, 1.25 μg, Panel B, 0.5 μg. Lanes 3-7, Panels A and B, contain 1.25 and 0.5 μg of yeast cell-lysate, respectively: lane 3, transformant containing only vector pGAL110; lane 4, transformant containing full-length ORF0657n (SEQ ID NO: 28 without the carboxyl His-Tag); lanes 5, 6, and 7, transformant 1-1 that contains piUC-S(−) which expresses mature ORF0657nH region (SEQ ID NO: 3); lane 7 contains a cell-lysate (of transformant 1-1) that was frozen and subsequently thawed. Lane 8 contains molecular size standards.

FIGS. 8A-8U provide examples of different nucleic acid sequences encoding for ORF0657n related polypeptides. FIG. 8A provides a nucleic acid sequence (SEQ ID NO: 29) encoding for SEQ ID NO: 2 plus a carboxyl His-Tag. FIG. 8B provides a nucleic acid sequence (SEQ ID NO: 30) encoding SEQ ID NO: 4 plus a carboxyl His-Tag. FIG. 8C provides a yeast optimized nucleic acid sequence (SEQ ID NO: 31) encoding SEQ ID NO: 28 without a carboxyl His-Tag. FIG. 8D provides a yeast optimized nucleic acid sequence (SEQ ID NO: 32) encoding SEQ ID NO: 3. FIG. 8E provides a yeast optimized nucleic acid (SEQ ID NO: 33) sequence encoding SEQ ID NO: 1. FIGS. 8F-8M provide yeast optimized nucleic acid sequences (SEQ ID NOs: 34, 35, 36, 37, 38, 39, 40, and 41) encoding SEQ ID NO: 7 containing an amino terminus methionine. FIGS. 8N-8U provide yeast optimized nucleic acid sequences (SEQ ID NOs: 46-53) encoding different ORF0657n related polypeptides based on SEQ ID NO: 17 or SEQ ID NO: 20.

FIG. 9 shows a Western blot comparing intracellular expression of ORF0657n related polypeptides in S. cerevisiae. Lanes 1 and 18, MW size standards. Lanes 2 and 3, 50 and 100 ng, respectively of purified ORF0657nH produced in E. coli (SEQ ID NO: 4 plus a carboxyl His-Tag) Lane 5 contains 500 ng protein of cell lysate from S. cerevisiae vector control transformant. Lanes 7, 8, and 9 contain 1.0, 2.0, and 4.0 μg cell lysate protein from S. cerevisiae transformant producing SEQ ID NO: 28 without the carboxyl His-Tag. Lanes 11 and 12 contain 50 and 100 ng protein, respectively, of cell lysate from S. cerevisiae transformant producing ORF0657nH (SEQ ID NO: 3). Lanes 14 and 15 contain 250 and 500 ng protein, respectively, of cell lysate from S. cerevisiae transformant producing ORF0657nG (SEQ ID NO: 44). Lane 17 contains 250 ng cell lysate protein from ORF0657nG (SEQ ID NO: 44 plus a carboxyl His-Tag) E. coli producer. Lanes 4, 6, 10, 13, and 16 are empty.

FIG. 10 illustrates protective immunity data for ORF0657n related polypeptides produced in E. coli and yeast. “OFR0657nH (E. coli)” corresponds to SEQ ID NO: 4 with a carboxyl His-Tag. “OFR0657nI (E. coli)” corresponds to SEQ ID NO: 5 with a carboxyl His-Tag. “OFR0657nC (E. coil)” corresponds to SEQ ID NO: 28. “OFR0657nH (yeast)” corresponds to SEQ ID NO: 3.

FIG. 11 shows an exemplary Western blot of intracellular expression of ORF0657nI in S. cerevisiae. Lanes 1 and 25: MW size standards. Lanes 2, 3, and 24: 25, 50, and 100 ng, respectively, of purified ORF0657nH region (SEQ ID NO: 4 with a carboxyl His-Tag) produced in E. coil. Lanes 4-23 contain protein cell lysate from yeast transformants. Lanes 13-21 represent duplicate cell-lysates prepared from the same fermentation sample as the lysates in lanes 4-12. Lanes 4 and 13 contain 200 ng protein from vector control transformant containing pGAL110. Lanes 5 and 14 contain 100 ng protein from transformant 1-1 which produces ORF0657nH region (SEQ ID NO: 3). Lanes 6 and 15 contain 200 ng protein from transformant 1-1. Lanes 7 and 16 contain 100 ng protein and lanes 8 and 17 contain 200 ng of protein from transformant I1. Lanes 9 and 18 contain 100 ng of protein and lanes 10 and 19 contain 200 ng protein from transformant I2. Lanes 11 and 20 contain 100 ng protein and lanes 12 and 21 contain 200 ng protein from transformant I3. Lanes 22 and 23 contain 100 and 200 ng protein cell-lysate prepared previously from a prior fermentation of transformant 1-1.

FIG. 12 provides Rhesus monkeys immunization data. Rhesus monkeys were immunized with either yeast produced ORF0657n related polypeptide (ORF0657nH, SEQ ID NO: 3) or E coli-expressed ORF0657n related polypeptide (full-length ORF0657nC, SEQ ID NO: 28) formulated with or without AHP. The monkeys in the vaccine group received 50 mcg ORF0657n related polypeptides via the intramuscular route.

### DETAILED DESCRIPTION OF THE INVENTION

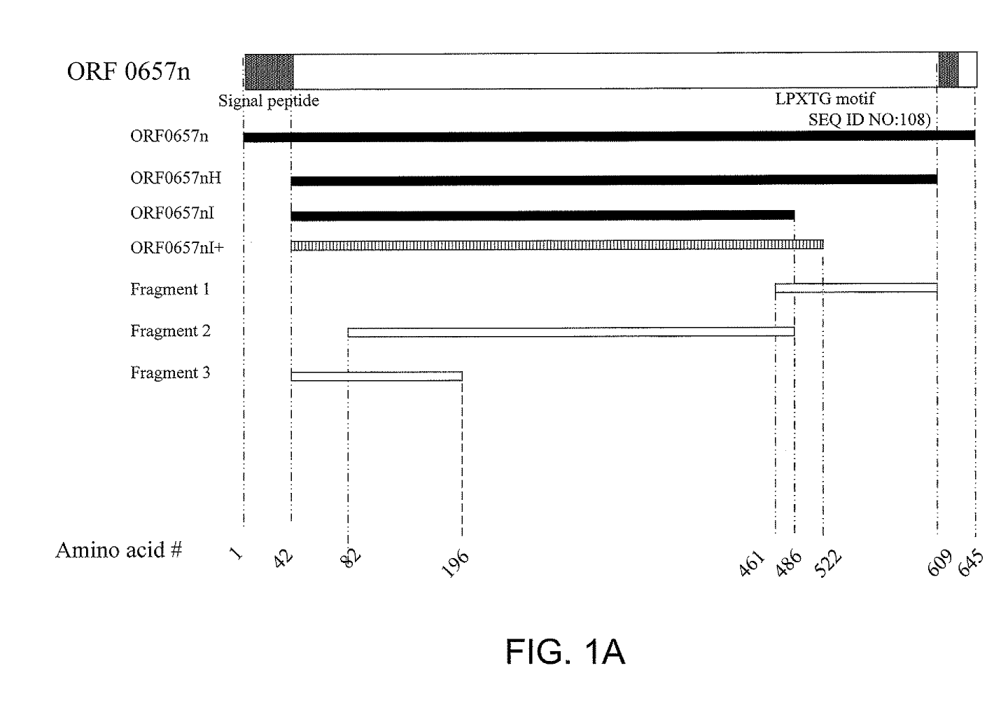

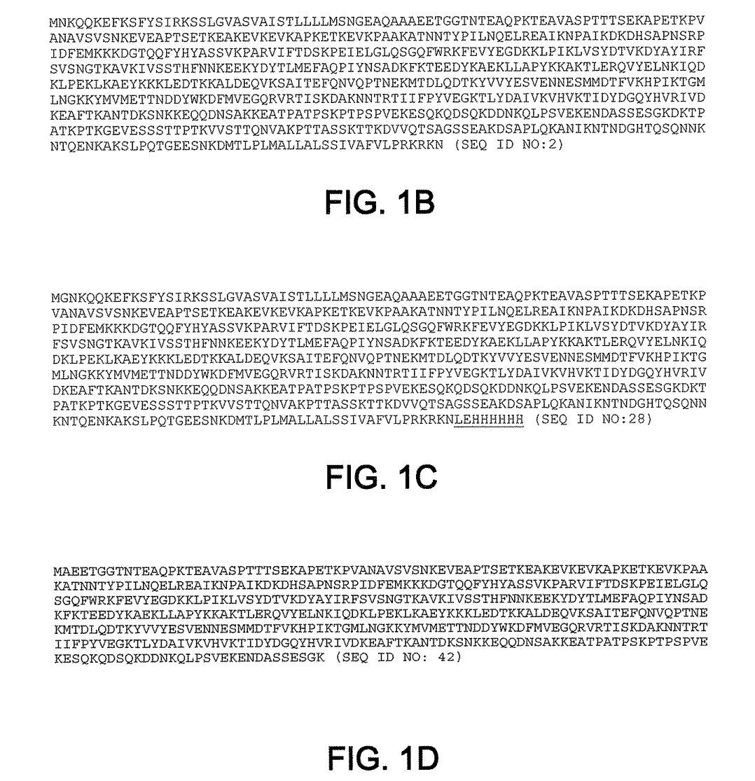

ORF0657n related polypeptides including full-length and shorter length derivatives containing the ORF0657nI region were found to provide protective immunity against S. aureus using an animal model. FIG. 1a illustrates the location of different ORF0657n related polypeptide regions providing protective immunity against an S. aureus infection and regions that did not provide protective immunity. In FIG. 1a, ORF0657n refers to a full-length sequence corresponding to SEQ ID NO: 2, ORF0657nI refers to a region corresponding to SEQ ID NO: 1 (without the amino terminus methionine), and ORF0657nH refers to a region corresponding to SEQ ID NO: 3 (without the amino terminus methionine).

An ORF0657n “related” polypeptide contains a region structurally related to a full-length ORF0657n or a fragment thereof. ORF0657n related polypeptides are polypeptides having at least about 90% sequence identity to a corresponding region of a naturally occurring ORFO657n. The reference ORF0657n illustrated in FIG. 1 corresponds to ORF0657n from S. aureus COL (SEQ ID NO: 2).

Percent identity to a reference sequence is determined by aligning the polypeptide sequence with the reference sequence and determining the number of identical amino acids. This number is divided by the total number of amino acids in the reference sequence and then multiplied by 100 and rounded to the nearest whole number.

FIG. 1a helps illustrate the importance of a core region comprising an amino acid sequence structurally related to SEQ ID NO: 1. SEQ ID NO: 1 comprises amino acids 42-486 of ORF0657n COL. SEQ ID NO: 1 also contains an amino terminus methionine to facilitate expression. Polypeptide fragments made up of SEQ ID NO: 2 amino acids 461-609, amino acids 82-486, or amino acids 42-196 were not protective.

Different amino acid and nucleic acid sequences are referred to throughout the application. Table 1 provides a summary of some of the polypeptide sequences indicating the FIG. 1 region and additional modifications. Table 2 provides a summary of some of the nucleic acid sequences.

TABLE 1

| | | Further modification/ |
|---|---|---|
| SEQ ID NOs: | ORF0657n region | additional information |
| 1 | ORF0657nI | Amino terminus methionine |
| 2 | Full-length | |
| 3 | ORF0657nH | Amino terminus methionine |
| 4 | ORF0657nH | Amino terminus methionine-glycine |
| 5 | ORF0657nI | Amino terminus methionine-glycine |
| 6 | ORF0657nH | |
| 7 | ORF0657nH | |
| 8 | ORF0657nH | |
| 9 | ORF0657nH | |
| 10 | ORF0657nH | |
| 11 | ORF0657nH | |
| 12 | ORF0657nH | |
| 13 | ORF0657nH | |
| 14 | ORF0657nH | |
| 15 | ORF0657nH | |
| 16 | ORF0657nH | |
| 17 | ORF0657nH | |
| 18 | ORF0657nH | |
| 19 | ORF0657nH | |
| 20 | ORF0657nH | |
| 21 | ORF0657nH | |
| 22 | ORF0657nH | |
| 23 | ORF0657nH | |
| 24 | ORF0657nH | |
| 25 | ORF0657nH | |
| 26 | ORF0657nH | |
| 27 | ORF0657nH | |
| 28 | Full-length | SEQ ID NO: 2 modified to contain a |
| | | glycine after the amino terminus |
| | | methionine and a carboxyl His-Tag |
| 42 | ORF0657nI⁺ | Amino acids 1-481 of SEQ ID NO: 3 |
| 44 | ORF0657nG | Amino terminus methionine plus |
| | | amino acids 42-645 of SEQ ID NO: 2 |
| 54-63 | ORF0657nH | From different S. aureus |
| 106 and 107 | Full-length | From different S. aureus |

TABLE 2

| SEQ ID NO: | Region | Other information |
|---|---|---|
| 29 | Full-length | Encodes SEQ ID NO: 2 (nucleotides 1-1935) + a carboxyl |
| | | His-Tag |
| 30 | ORF0657nH | Encodes SEQ ID NO: 4 (nucleotides 1-1710) + a carboxyl |
| | | His-Tag |
| 31 | Full-length | Encodes SEQ ID NO: 28 without a carboxyl His-Tag and is |
| | | codon optimized for yeast expression (amino acids 1-646 of |
| | | SEQ ID NO: 28) |
| 32 | ORF0657nH | Encodes SEQ ID NO: 3 and is codon optimized for yeast |
| | | expression |
| 33 | ORF0657nI | Encodes SEQ ID NO: 1 and is codon optimized for yeast |
| | | expression |
| 34 | ORF0657nH | Encodes SEQ ID NO: 7 containing an amino terminus |
| | | methionine and is codon optimized for yeast expression |
| 35 | ORF0657nH | Encodes SEQ ID NO: 7 containing an amino terminus |
| | | methionine and is codon optimized for yeast expression |
| 36 | ORF0657nH | Encodes SEQ ID NO: 7 containing an amino terminus |
| | | methionine and is codon optimized for yeast expression |
| 37 | ORF0657nH | Encodes SEQ ID NO: 7 containing an amino terminus |
| | | methionine and is codon optimized for yeast expression |
| 38 | ORF0657nH | Encodes SEQ ID NO: 7 containing an amino terminus |
| | | methionine and is codon optimized for yeast expression |
| 39 | ORF0657nH | Encodes SEQ ID NO: 7 containing an amino terminus |
| | | methionine and is codon optimized for yeast expression |
| 40 | ORF0657nH | Encodes SEQ ID NO: 7 containing an amino terminus |
| | | methionine and is codon optimized for yeast expression |
| 41 | ORF0657nH | Encodes SEQ ID NO: 7 containing an amino terminus |
| | | methionine and is codon optimized for yeast expression |
| 43 | ORF0657nI⁺ | Encodes SEQ ID NO: 42 and is codon optimized for yeast |
| | | expression |
| 45 | ORF0657nG | Encodes SEQ ID NO: 44 containing an amino terminus |
| | | methionine and is codon optimized for yeast expression |
| 46 | ORF0657nH | Encodes SEQ ID NO: 17 containing an amino terminus |
| | | methionine and is codon optimized for yeast expression |
| 47 | ORF0657nI⁺ | Encodes the SEQ ID NO: 17 I⁺ region, is codon optimized |
| | | for yeast expression, and encodes a methionine initiation |
| | | codon |
| 48 | ORF0657nI | Encodes the SEQ ID NO: 17 I region, is codon optimized for |
| | | yeast expression, and encodes a methionine initiation codon |
| 49 | Full-length | Encodes for full length ORF0657n containing SEQ ID NO: |
| | | 17 (SEQ ID NO: 106) modified to contain a glycine after the |
| | | amino terminus methionine and is codon optimized for yeast |
| | | expression |
| 50 | ORF0657nH | Encodes SEQ ID NO: 20, is codon optimized for yeast |
| | | expression, and encodes a methionine initiation codon |
| 51 | ORF0657nI⁺ | Encodes SEQ ID NO: 20 I⁺ region, is codon optimized for |
| | | yeast expression, and encodes a methionine initiation codon |
| 52 | ORF0657nI | Encodes the SEQ ID NO: 20 I region, is codon optimized for |
| | | yeast expression, and encodes a methionine initiation codon |
| 53 | Full length | Encodes for full length ORF0657n containing SEQ ID NO: |
| | | 20 (SEQ ID NO: 107) modified to contain a glycine after the |
| | | amino terminus methionine and is codon optimized for yeast |
| | | expression |

### SEQ ID NO: 1 Related Polypeptides

A polypeptide region structurally related to SEQ ID NO: 1 contains an amino acid identity of at least 90% to SEQ ID NO: 1. Polypeptides containing a region structurally related to SEQ ID NO: 1 can be designed based on the guidance provided herein to obtain polypeptides protective against S. aureus.

Using SEQ ID NO: 1 as a frame of reference, alterations can be made taking into account the amino acid sequence of different naturally occurring ORF0657n polypeptides and known properties of amino acids. Alterations include one or more amino acid additions, deletions, and/or substitutions. The overall effect of different alterations can be evaluated using techniques described herein to confirm the ability of a particular polypeptide to provide protective immunity.

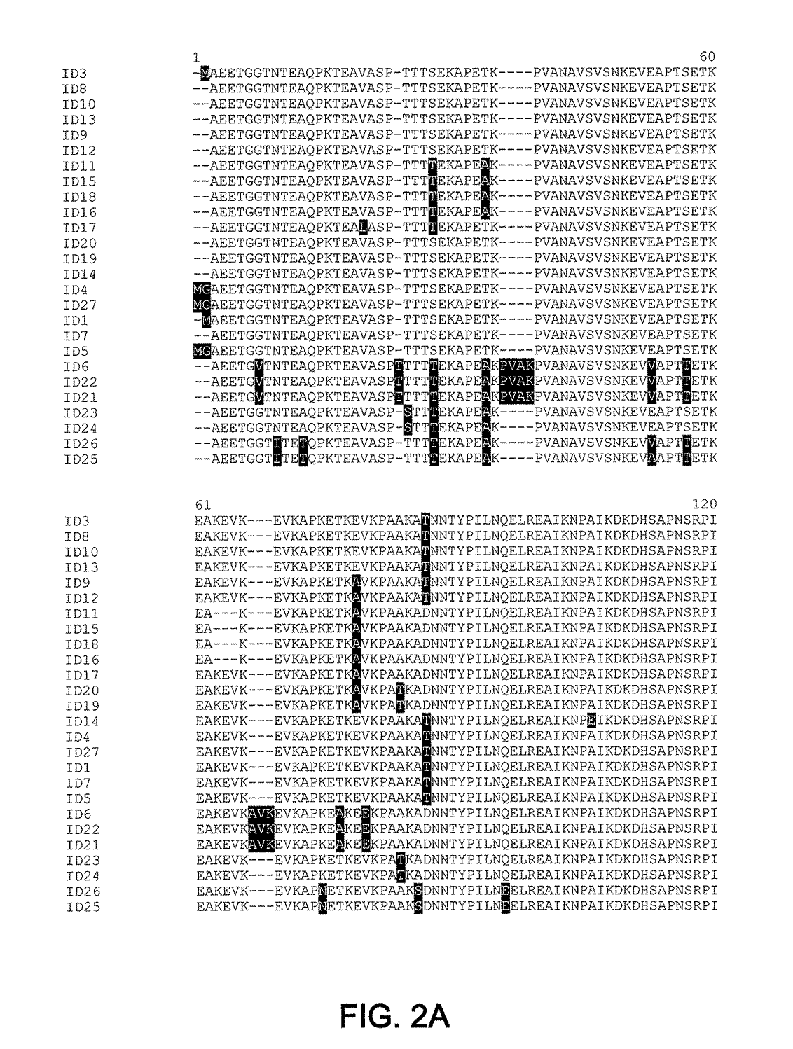

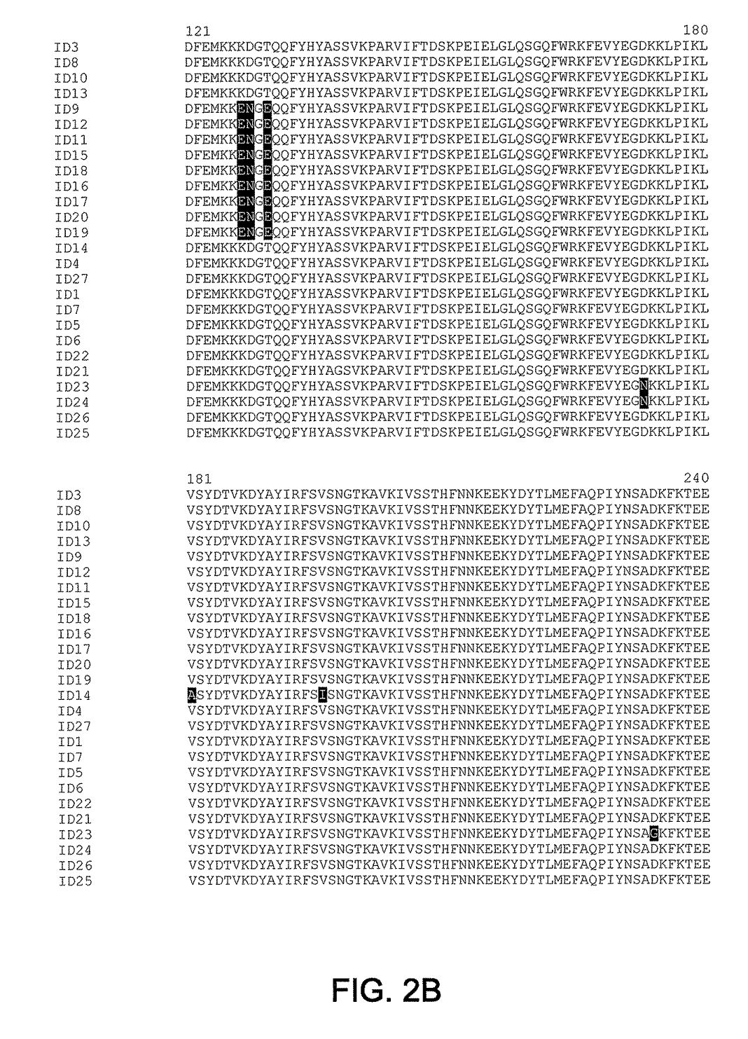

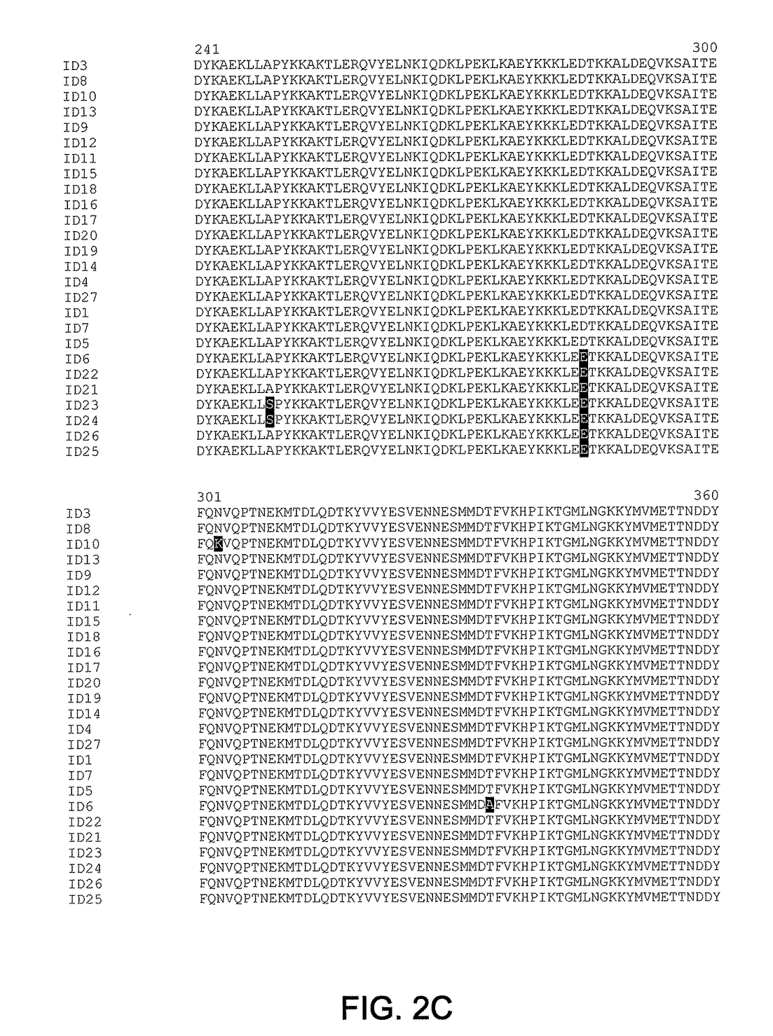

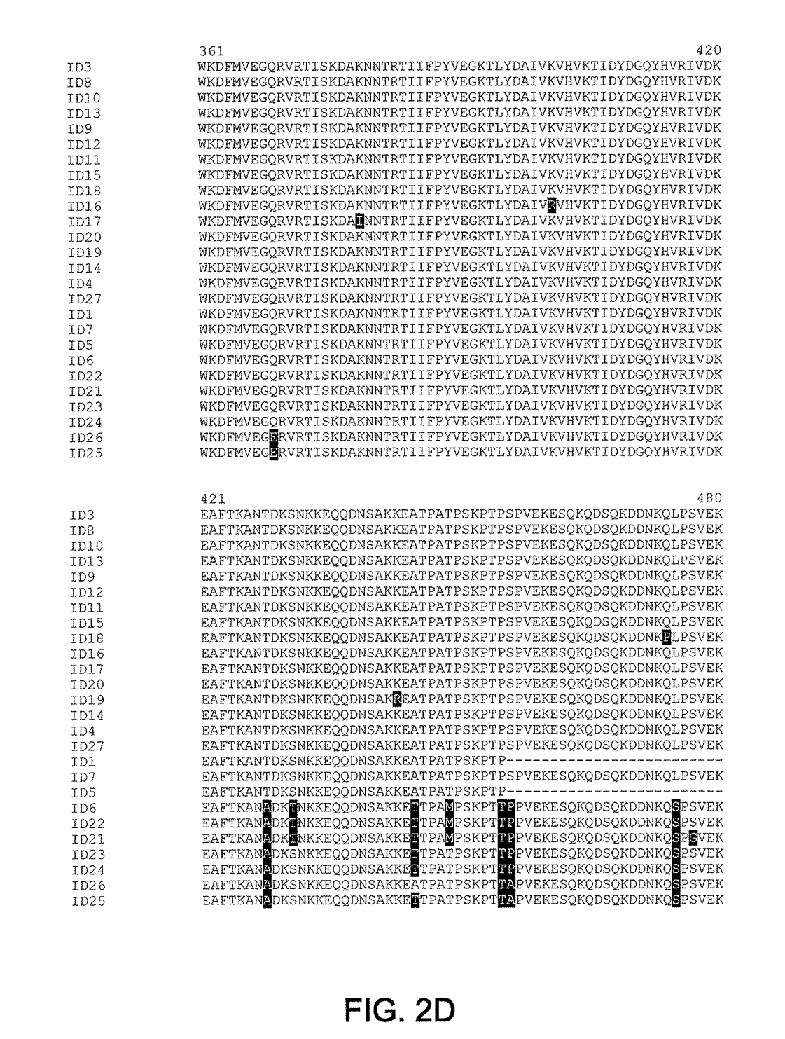

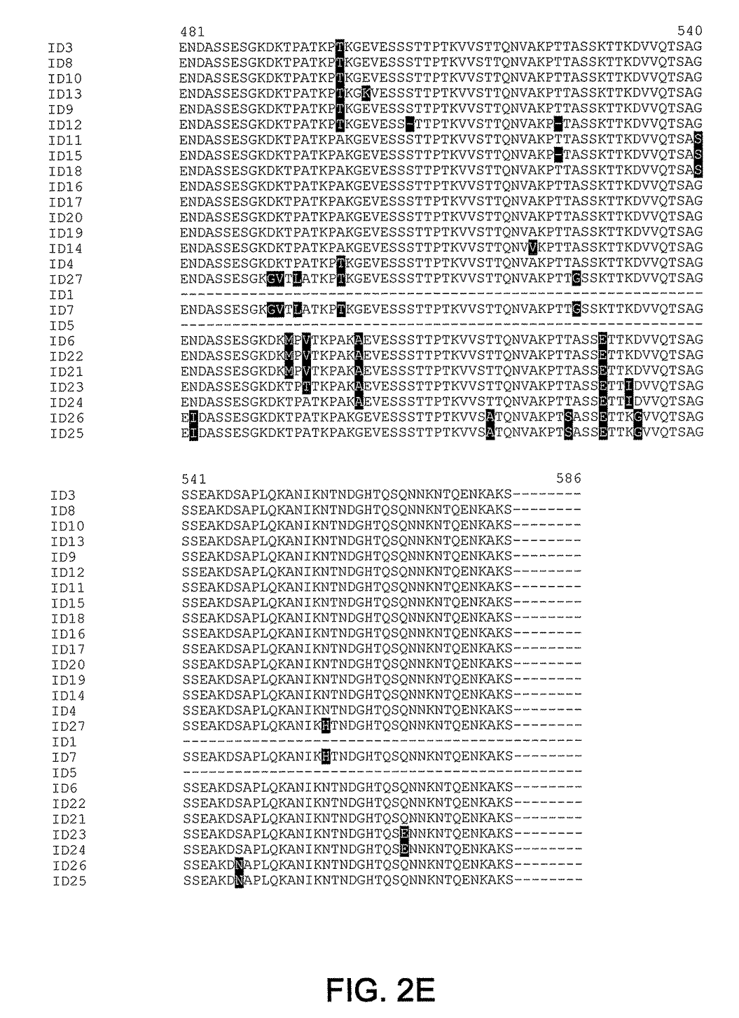

ORF0657n was found to be well conserved across a collection of pathologically and taxonomically diverse S. aureus clinical isolates. (See Example 5 infra.) FIG. 2 provides an amino acid sequence comparison for different sequences containing a SEQ ID NO: 1. The illustrated sequence comparison is for the ORF0657nH region. The ORF0657nH region includes the smaller protective ORF0657nI region.

FIG. 2 provides a sequence comparison of SEQ ID NO: 1 and 3-27. The comparison illustrates amino acid differences between S. aureus clinical isolates that can be used to guide the design of potential alterations to S. aureus related polypeptides such as SEQ ID NOs: 1 and 3. In addition, alterations can be made taking into account known properties of amino acids. SEQ ID NOs: 1, 3-6 and 8-26 illustrate naturally occurring sequences that start at position number 3, position numbers 1 and 2 illustrate the addition of methionine or methionine-glycine to the amino terminus to some sequences. SEQ ID NOs: 11-26 were obtained from different clinical isolates. SEQ ID NOs: 7 and 27 are variants of SEQ ID NO: 4 ORF0657nH region that contains five amino acid substitutions in a region outside the SEQ ID NO: 1 core region.

Additional ORF0657n sequences can be used in the sequence comparison to help guide alterations. Examples of additional S. aureus ORF0657nH region sequences are provided by SEQ ID NOs: 54-63, the full length sequence for SEQ ID NOs: 17 and 20 are provided by SEQ ID NOs: 106 and 107.

Generally, in substituting different amino acids to retain activity it is preferable to exchange amino acids having similar properties. Factors that can be taken into account for an amino acid substitution include amino acid size, charge, polarity, and hydrophobicity. The effect of different amino acid R-groups on amino acid properties are well known in the art. (See, for example, Ausubel, Current Protocols in Molecular Biology, John Wiley, 1987-2002, Appendix 1C.)

In exchanging amino acids to maintain activity, the replacement amino acid should have one or more similar properties such as approximately the same charge and/or size and/or polarity and/or hydrophobicity. For example, substituting valine for leucine, arginine for lysine, and asparagine for glutamine are good candidates for not causing a change in polypeptide function.

Alterations to achieve a particular purpose include those designed to facilitate production or efficacy of the polypeptide; or cloning of the encoded nucleic acid. Polypeptide production can be facilitated through the use of an initiation codon (e.g., coding for methionine) suitable for recombinant expression. The methionine may be later removed during cellular processing. Cloning can be facilitated by, for example, the introduction of restriction sites which can be accompanied by amino acid additions or changes.

Efficacy of a polypeptide to induce an immune response can be enhanced through epitope enhancement. Epitope enhancement can be performed using different techniques such as those involving alteration of anchor residues to improve peptide affinity for MHC molecules and those increasing affinity of the peptide-MHC complex for a T-cell receptor. (Berzofsky et al., Nature Review 1:209-219, 2001.)

In different embodiments, with respect to the SEQ ID NO: 1 related polypeptide region, the region is at least 90%, at least 94%, or at least 99% identical to SEQ ID NO: 1; differs from SEQ ID NO: 1 by 0, 1, 2, 3, 4, 5, 6, 7, 8, 9, 10, 11, 12, 13, 14, 15, 16, 17, 18, 19, 20, 21, 22, 23, 24, or 25 alterations, or up to 50 alterations; or consists essentially or consists of an OFR0657nI related region selected from the group consisting of:

amino acids 1-442 of SEQ ID NOs: 11, 15, 16, 18, or 54;
amino acids 1-443 of SEQ ID NO: 63
amino acids 1-444 of SEQ ID NOs: 57 or 59;
amino acids 1-445 of SEQ ID NOs: 7, 8, 9, 10, 12, 13, 14, 17, 19, 20, 55, 56 or 58;
amino acids 1-446 of SEQ ID NOs: 23 or 24;
amino acids 1-446 or 2-446 of: SEQ ID NOs: 1 or 3;
amino acids 1-447 of SEQ ID NOs: 25, or 26; or
amino acids 1-447, 2-447, or 3-447 of SEQ ID NOs: 4, 5, or 27;
amino acids 1-449 of SEQ ID NOs: 61 or 62;
amino acids 1-453 of SEQ ID NO: 60; and
amino acids 1-454 of SEQ ID NOs: 6, 21, or 22.

Reference to “consists essentially” of indicated amino acids indicates that the referred to amino acids are present and additional amino acids may be present. The additional amino acids can be at the carboxyl or amino terminus. In different embodiments 1, 2, 3, 4, 5, 6, 7, 8, 9, 10, 11, 12, 13, 14, 15, 16, 17, 18, 19, or 20 additional amino acids are present. In preferred embodiments methionine is present at the amino terminus; or methionine-glycine is present at the amino terminus.

In an embodiment of the present invention, the polypeptide consists of an amino acid sequence at least 90% identical to SEQ ID NO: 42 or a fragment thereof comprising an amino acid sequence structurally related to SEQ ID NO: 1. In different embodiments, with respect to SEQ ID NO: 42, the polypeptide is at least 94% or at least 99% identical to SEQ ID NO: 42; differs from SEQ ID NO: 42 by 0, 1, 2, 3, 4, 5, 6, 7, 8, 9, 10, 11, 12, 13, 14, 15, 16, 17, 18, 19, 20, 21, 22, 23, 24, or 25 alterations, up to 50 alterations, or up to 65 alterations; consists or consists essentially of SEQ ID NO: 42 or an OFR0657nI⁺ related region selected from the group consisting of:

amino acids 1-477 of SEQ ID NOs: 11, 15, 16, 18, or 54;
amino acids 1-478 of SEQ ID NO: 63
amino acids 1-479 of SEQ ID NOs: 57 or 59;
amino acids 1-480 of SEQ ID NOs: 7, 8, 9, 10, 12, 13, 14, 17, 19, 20, 55, 56, or 58;
amino acids 1-481 of SEQ ID NOs: 23 or 24;
amino acids 1-481 or 2-481 of SEQ ID NOs: 1 or 3;
amino acids 1-482 of SEQ ID NOs: 25 or 26;
amino acids 1-482, 2-482, or 3-482 of SEQ ID NOs: 4, 5, or 27;
amino acids 1-484 of SEQ ID NOs: 61 or 62;
amino acids 1-488 of SEQ ID NO: 60; and
amino acids 1-489 of SEQ ID NOs: 6, 21, or 22;

In another embodiment of the present invention, the polypeptide consists of an amino acid sequence at least 90% identical to SEQ ID NO: 3 or a fragment thereof comprising an amino acid sequence structurally related to SEQ ID NO: 1. In different embodiments, with respect to SEQ ID NO: 3, the polypeptide is at least 94%, or at least 99% identical to SEQ ID NO: 3; differs from SEQ ID NO: 3 by 0, 1, 2, 3, 4, 5, 6, 7, 8, 9, 10, 11, 12, 13, 14, 15, 16, 17, 18, 19, 20, 21, 22, 23, 24, or 25 alterations, up to 50 alterations, or up to 65 alterations; or consists or consists essentially of an amino acid sequence selected from the group consisting of SEQ ID NOs: 3, 4, 6, 7, 8, 9, 10, 11, 12, 13, 14, 15, 16, 17, 18, 19, 20, 21, 22, 23, 24, 25, 26, 27, 54, 55, 56, 57, 58, 59, 60, 61, 62, and 63.

In an additional embodiment the polypeptide consists or consists essentially of a polypeptide of SEQ ID NO: 2 modified by the insertion of a glycine after the initial methionine, or such a polypeptide without the initial methionine.

In an additional embodiment the polypeptide is a purified polypeptide. A “purified polypeptide” is present in an environment lacking one or more other polypeptides with which it is naturally associated and/or is represented by at least about 10% of the total protein present. In different embodiments, the purified polypeptide represents at least about 50%, at least about 75%, or at least about 95% of the total protein in a sample or preparation.

In an additional embodiment the polypeptide is “substantially purified.” A substantially purified polypeptide is present in an environment lacking all, or most, other polypeptides with which the polypeptide is naturally associated. For example, a substantially purified S. aureus polypeptide is present in an environment lacking all, or most, other S. aureus polypeptides. An environment can be, for example, a sample or preparation.

Reference to “purified” or “substantially purified” does not require a polypeptide to undergo any purification and may include, for example, a chemically synthesized polypeptide that has not been purified.

Polypeptide stability can be enhanced by modifying the polypeptide carboxyl or amino terminus. Examples of possible modifications include amino terminus protecting groups such as acetyl, propyl, succinyl, benzyl, benzyloxycarbonyl or t-butyloxycarbonyl; and carboxyl terminus protecting groups such as amide, methylamide, and ethylamide.

In an embodiment of the present invention the protective polypeptide is part of an immunogen consisting of the polypeptide and one or more additional regions or moieties covalently joined to the polypeptide at the carboxyl terminus or amino terminus. Each region or moiety should be independently selected from a region or moiety having at least one of the following properties: enhances the immune response, facilitates purification, or facilitates polypeptide stability. Polypeptide stability can be enhanced, for example, using groups such as polyethylene glycol that may be present on the amino or carboxyl terminus.

Polypeptide purification can be enhanced by adding a group to the carboxyl or amino terminus to facilitate purification. Examples of groups that can be used to facilitate purification include polypeptides providing affinity tags. Examples of affinity tags include a six-histidine tag, trpE, glutathione and maltose-binding protein.

The ability of a polypeptide to produce an immune response can be enhanced using groups that generally enhance an immune response. Examples of groups that can be joined to a polypeptide to enhance an immune response against the polypeptide include cytokines such as IL-2. (Buchan et al., 2000. Molecular Immunology 37:545-552.)

### Polypeptide Production

Polypeptides can be produced using standard techniques including those involving chemical synthesis and those involving purification from a cell producing the polypeptide. Techniques for chemical synthesis of polypeptides are well known in the art. (See e.g., Vincent, Peptide and Protein Drug Delivery, New York, N.Y., Decker, 1990.)

Techniques for polypeptide purification from a cell are illustrated in the Examples provided below. Additional examples of purification techniques are well known in the art. (See for example, Ausubel, Current Protocols in Molecular Biology, John Wiley, 1987-2002.)

Obtaining polypeptides from a cell is facilitated using recombinant nucleic acid techniques to produce the polypeptide. Recombinant nucleic acid techniques for producing a polypeptide involve introducing, or producing, a recombinant gene encoding the polypeptide in a cell and expressing the polypeptide.

A recombinant gene contains nucleic acid encoding a polypeptide along with regulatory elements for polypeptide expression. The recombinant gene can be present in a cellular genome or can be part of an expression vector.

The regulatory elements that may be present as part of a recombinant gene include those naturally associated with the polypeptide encoding sequence and exogenous regulatory elements not naturally associated with the polypeptide encoding sequence. Exogenous regulatory elements such as an exogenous promoter can be useful for expressing a recombinant gene in a particular host or increasing the level of expression. Generally, the regulatory elements that are present in a recombinant gene include a transcriptional promoter, a ribosome binding site, a terminator, and an optionally present operator. A preferred element for processing in eukaryotic cells is a polyadenylation signal.

Expression of a recombinant gene in a cell is facilitated through the use of an expression vector. Preferably, an expression vector in addition to a recombinant gene also contains an origin of replication for autonomous replication in a host cell, a selectable marker, a limited number of useful restriction enzyme sites, and a potential for high copy number. Examples of expression vectors are cloning vectors, modified cloning vectors, specifically designed plasmids and viruses.

Due to the degeneracy of the genetic code, a large number of different encoding nucleic acid sequences can be used to code for a particular polypeptide. The degeneracy of the genetic code arises because almost all amino acids are encoded by different combinations of nucleotide triplets or “codons”. Amino acids are encoded by codons as follows:

A=Ala=Alanine: codons GCA, GCC, GCG, GCU
C=Cys=Cysteine: codons UGC, UGU
D=Asp=Aspartic acid: codons GAC, GAU
E=Glu=Glutamic acid: codons GAA, GAG
F=Phe=Phenylalanine: codons UUC, UUU
G=Gly=Glycine: codons GGA, GGC, GGG, GGU
H=His=Histidine: codons CAC, CAU
I=Ile=Isoleucine: codons AUA, AUC, AUU
K=Lys=Lysine: codons AAA, AAG
L=Leu=Leucine: codons UUA, UUG, CUA, CUC, CUG, CUU
M=Met=Methionine: codon AUG
N=Asn=Asparagine: codons AAC, AAU
P=Pro=Proline: codons CCA, CCC, CCG, CCU
Q=Gln=Glutamine: codons CAA, CAG
R=Arg=Arginine: codons AGA, AGG, CGA, CGC, CGG, CGU
S=Ser=Serine: codons AGC, AGU, UCA, UCC, UCG, UCU
T=Thr=Threonine: codons ACA, ACC, ACG, ACU
V=Val=Valine: codons GUA, GUC, GUG, GUU
W=Trp=Tryptophan: codon UGG
Y=Tyr=Tyrosine: codons UAC, UAU

Suitable cells for recombinant nucleic acid expression of ORF0657n related polypeptides are prokaryotes and eukaryotes. Examples of prokaryotic cells include E. coli; members of the Staphylococcus genus, such as S. aureus; members of the Lactobacillus genus, such as L. plantarum; members of the Lactococcus genus, such as L. lactis; and members of the Bacillus genus, such as B. subtilis. Examples of eukaryotic cells include mammalian cells; insect cells; yeast cells such as members of the Saccharomyces genus (e.g., S. cerevisiae), members of the Pichia genus (e.g., P. pastoris), members of the Hansenula genus (e.g., H. polymorpha), members of the Kluyveromyces genus (e.g., K. lactis or K. fragilis) and members of the Schizosaccharomyces genus (e.g., S. pombe).

Techniques for recombinant gene production, introduction into a cell, and recombinant gene expression are well known in the art. Examples of such techniques are provided in references such as Ausubel, Current Protocols in Molecular Biology, John Wiley, 1987-2002, and Sambrook et al., Molecular Cloning, A Laboratory Manual, 2^{nd} Edition, Cold Spring Harbor Laboratory Press, 1989.

If desired, expression in a particular host can be enhanced through codon optimization. Codon optimization includes use of more preferred codons. Techniques for codon optimization in different hosts are well known in the art.

ORFO657n related polypeptides may contain post translational modifications, for example, N-linked glycosylation, O-linked glycosylation, or acetylation. Reference to “polypeptide” or an “amino acid” sequence of a polypeptide includes polypeptides containing one or more amino acids having a structure of a post-translational modification from a host cell, such as a mammalian, insect or yeast host cell.

Post translational modifications can be produced chemically or by making use of suitable hosts. For example, in S. cerevisiae the nature of the penultimate amino acid appears to determine whether the N-terminal methionine is removed. Furthermore, the nature of the penultimate amino acid also determines whether the N-terminal amino acid is N^{α}-acetylated (Huang et al., Biochemistry 26:8242-8246, 1987). Another example includes a polypeptide targeted for secretion due to the presence of a secretory leader (e.g., signal peptide), where the polypeptide is modified by N-linked or O-linked glycosylation. (Kukuruzinska et al., Ann. Rev. Biochem. 56:915-944, 1987.)

### Yeast Expression

ORF0657n related polypeptides are preferably expressed in yeast using encoding nucleic acid containing codons optimized for yeast expression. Expression in yeast can be performed using a recombinant gene encoding a ORF0657n related polypeptide and regulatory regions for yeast expression. Depending upon the employed expression system produced protein can remain intracellular or can be excreted outside the cell.

Preferably, the promoter for recombinant gene expression is an inducible promoter, such as a promoter from the yeast galactose gene cluster (a GAL promoter such as GALL, GAL7, GAL10, MEL1) or the acid phosphatase PHO5 promoter or the alcohol dehydrogenase II ADH2 promoter or the copper-regulated metallothionein CUP1 promoter. Examples of “constitutive” promoters that might be used are GAP (TDB), PGK or TPI promoters. (Romanos et al., YEAST 8:423-488, 1992.)

The yeast host cell used for recombinant expression can be selected or engineered to facilitate recombinant gene expression. Mutation such as mnn9, prb1 and/or pep4 mutations are typically desirable. To enhance expression from GAL promoters, over expression of GAL4 transcription factor can be achieved (Hopper et al., U.S. Pat. No. 5,068,185).

Codon optimization for a particular host is performed by replacing codons having a low or moderate usage level with codons having a high expression level. The percentage of optimal codons present in an encoding sequence can vary. In different embodiments the number of optimal codons (including codons initially present and codons introduced) is at least 50%, at least 75%, at least 95%, or 100% of the total number of codons.

Codon optimization can be performed as follows:

1. For a particular codon, compare the wild-type codon frequency to overall codon frequency of use by yeast genes.

2. If the codon is not one of those commonly employed by yeast, replace it with an optimal codon for high expression in yeast cells.

3. Repeat steps (1) and (2) for different codons until achieving the desired level of codon optimization.

4. Inspect the new coding sequence for undesired sequences generated such as unwanted restriction enzyme sites, splice sites, promoters, undesirable palindrome or repeat sequences, transcription terminator sequences, and high frequency of GC bases. Remove undesired sequences using an alternative codon.

Alternative codon usage is provided by Lathe, J. Molec. Biol., 183:1-12, 1985. Codon usage in different yeast hosts is well known in the art. For example, Sharp et al., Yeast 7:657-678, 1991, describes synonymous codon usage in Saccharomyces cerevisiae.

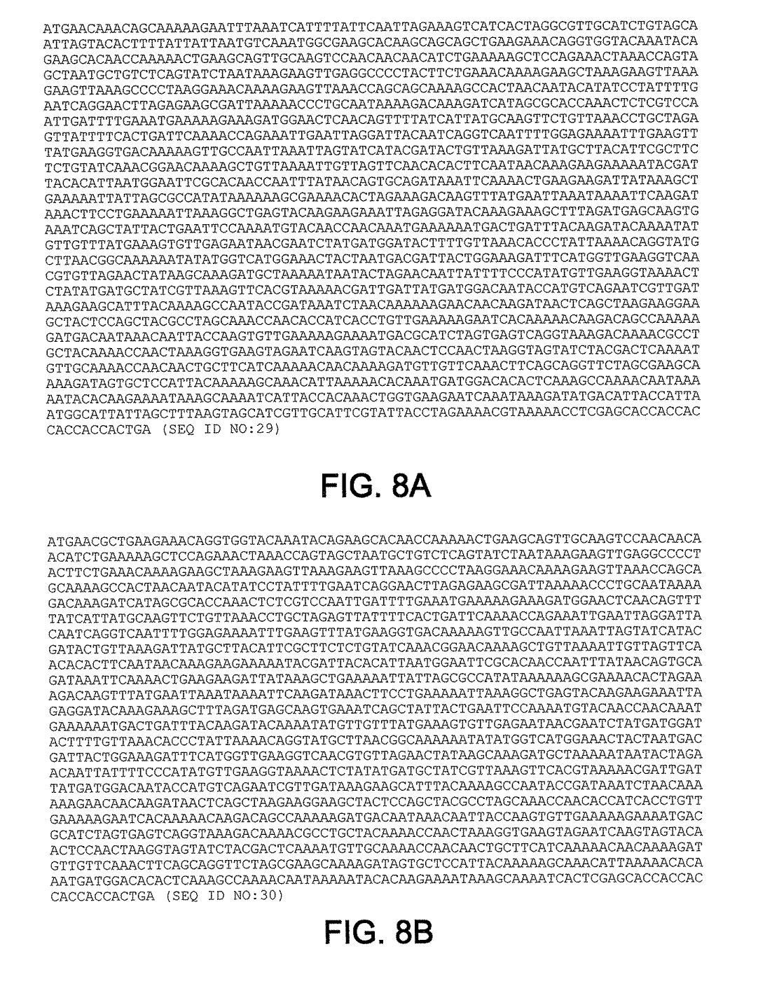

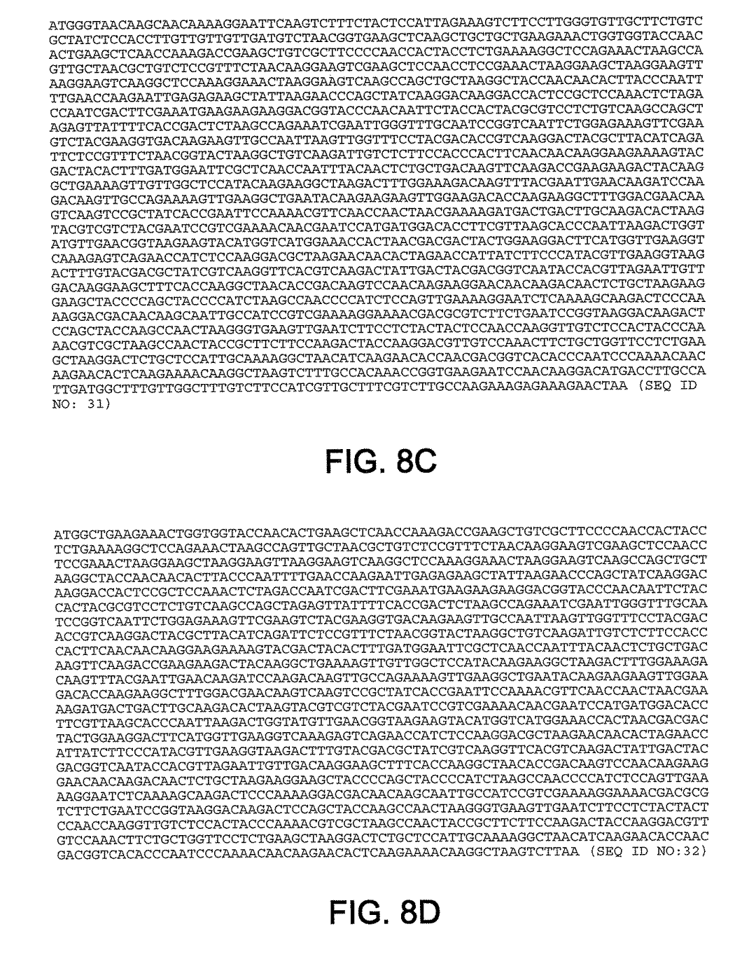

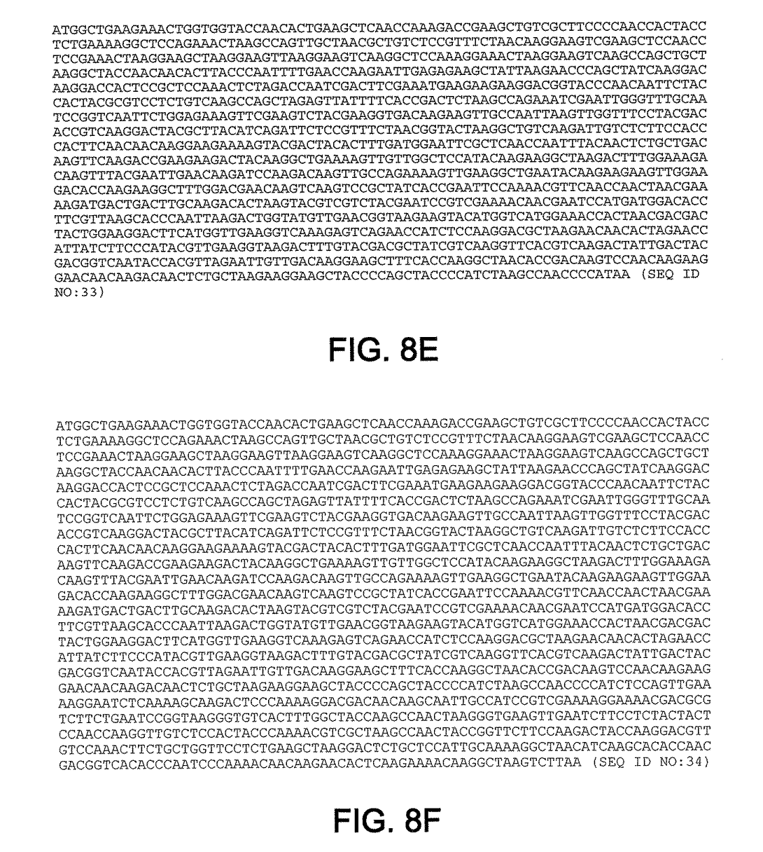

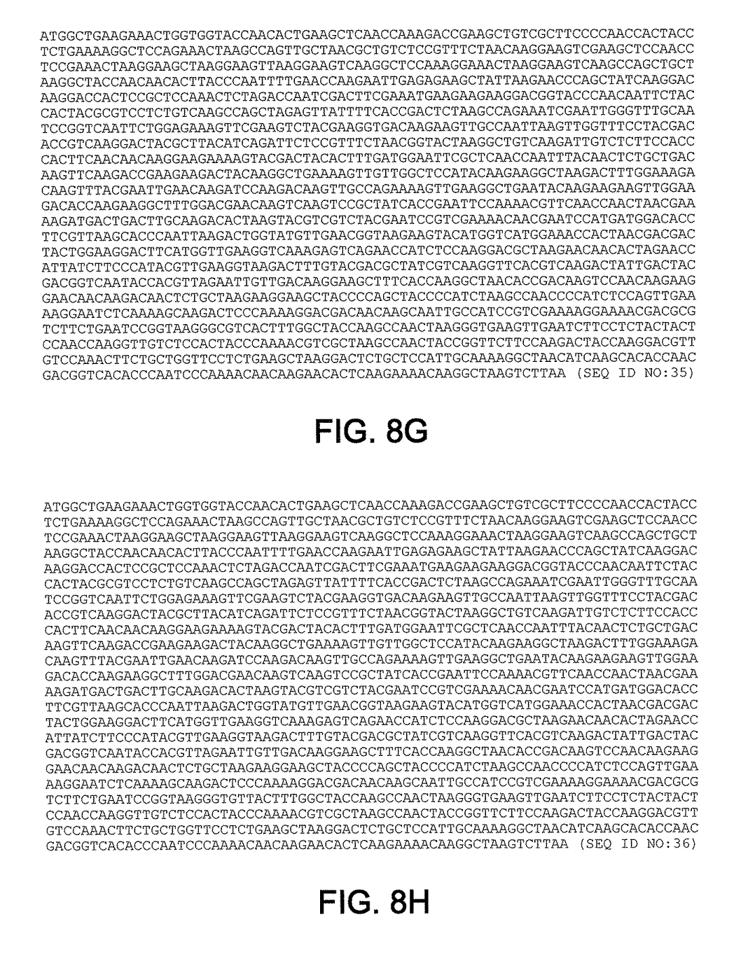

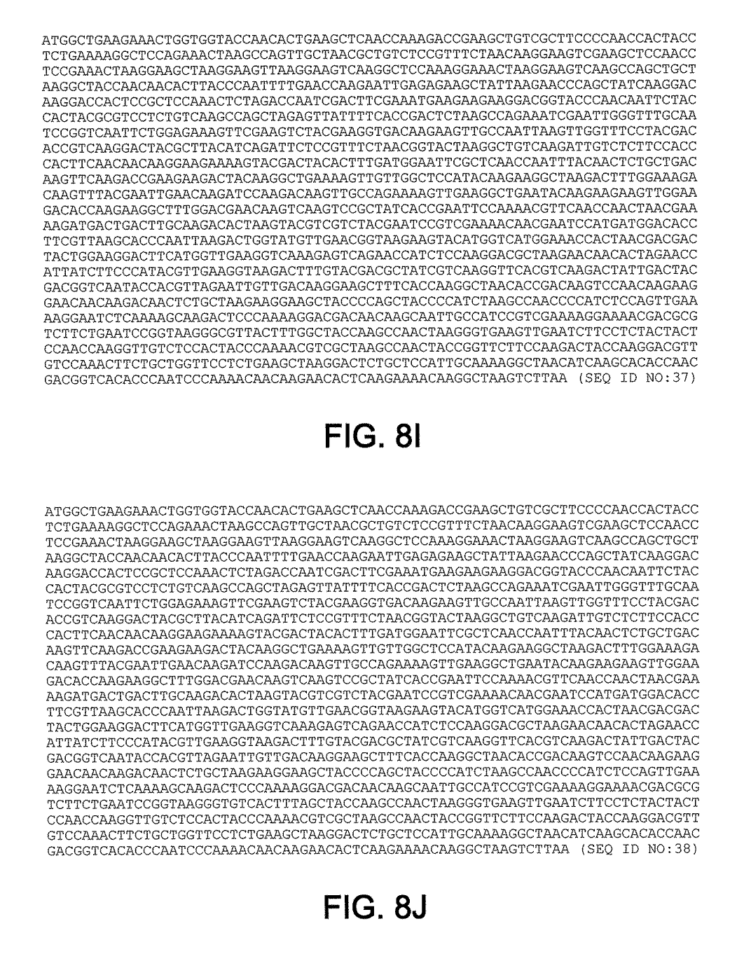

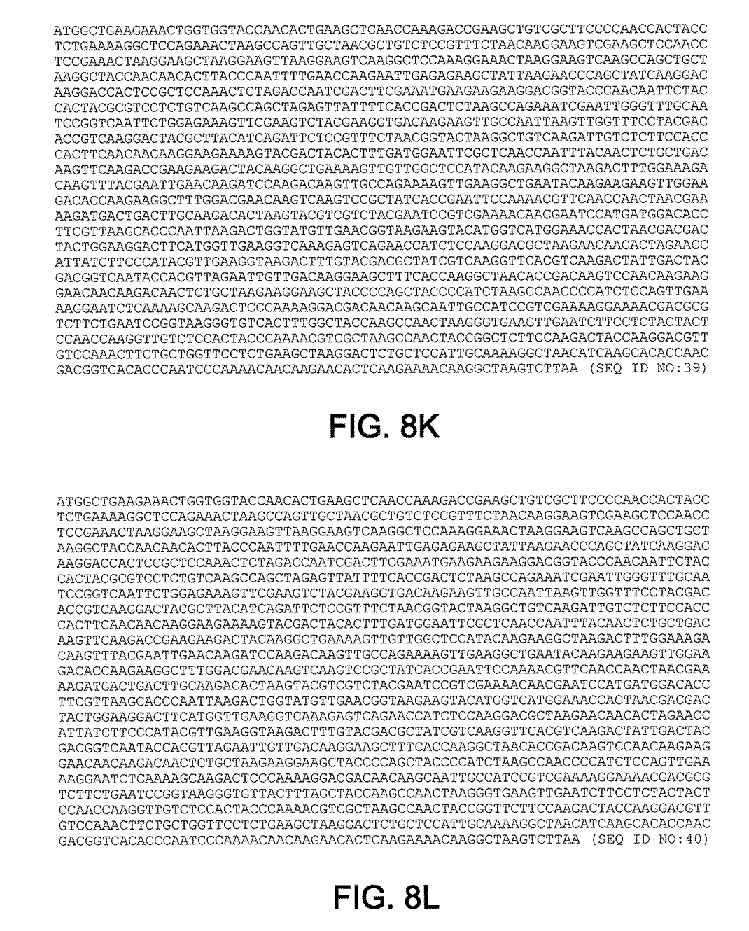

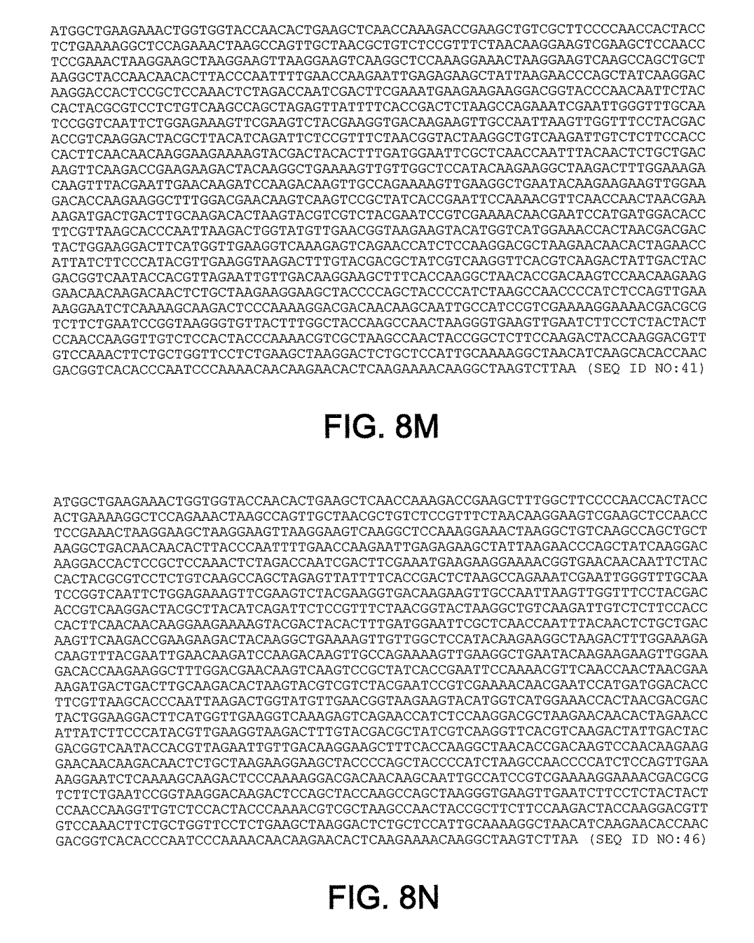

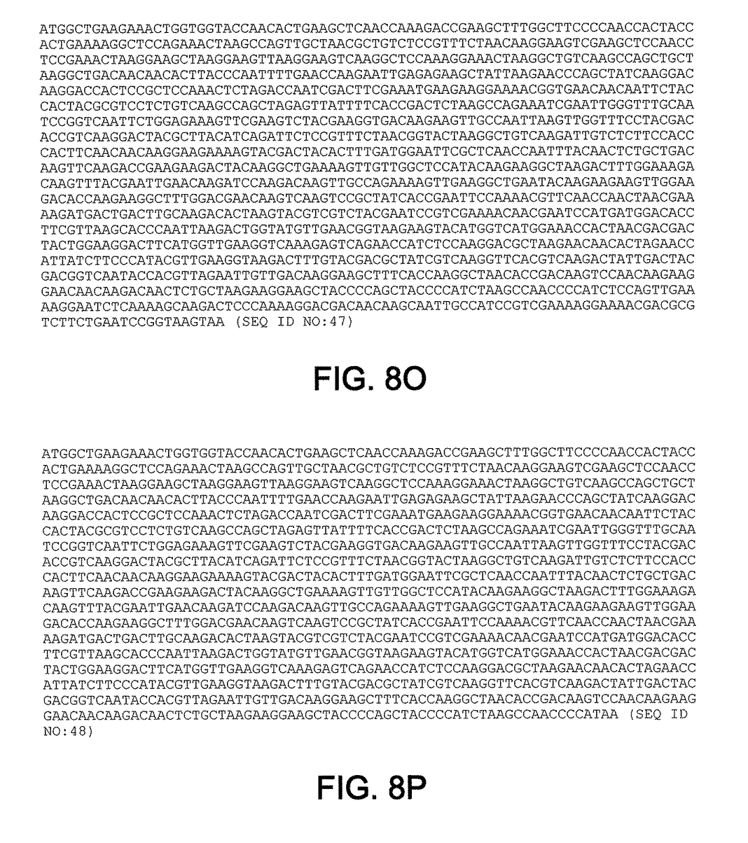

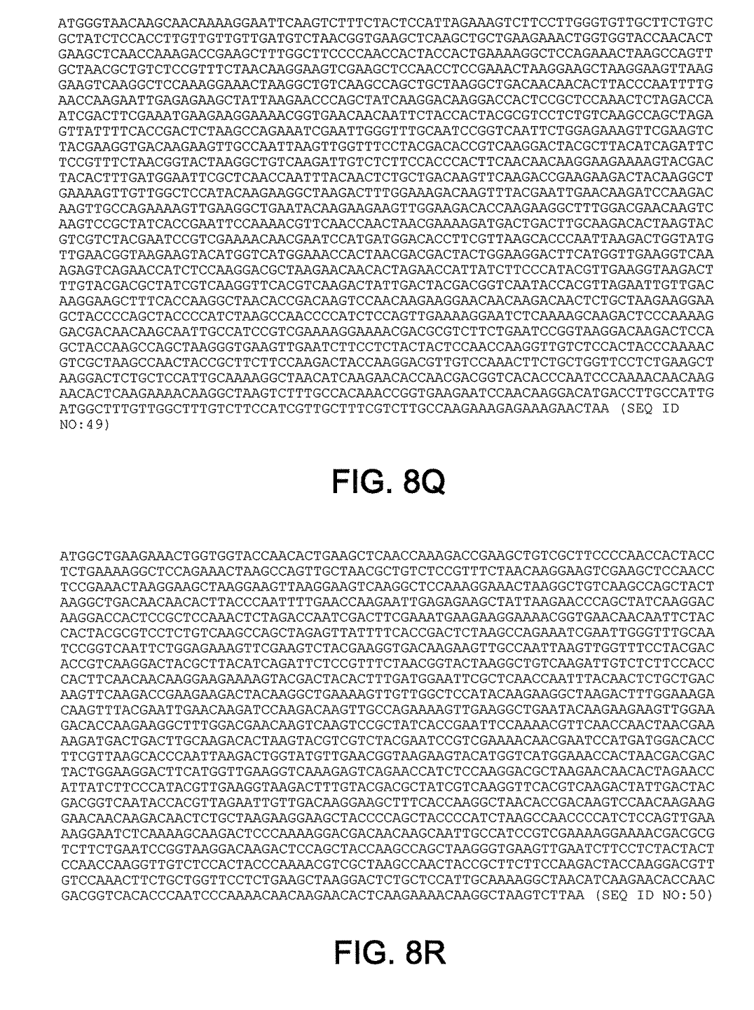

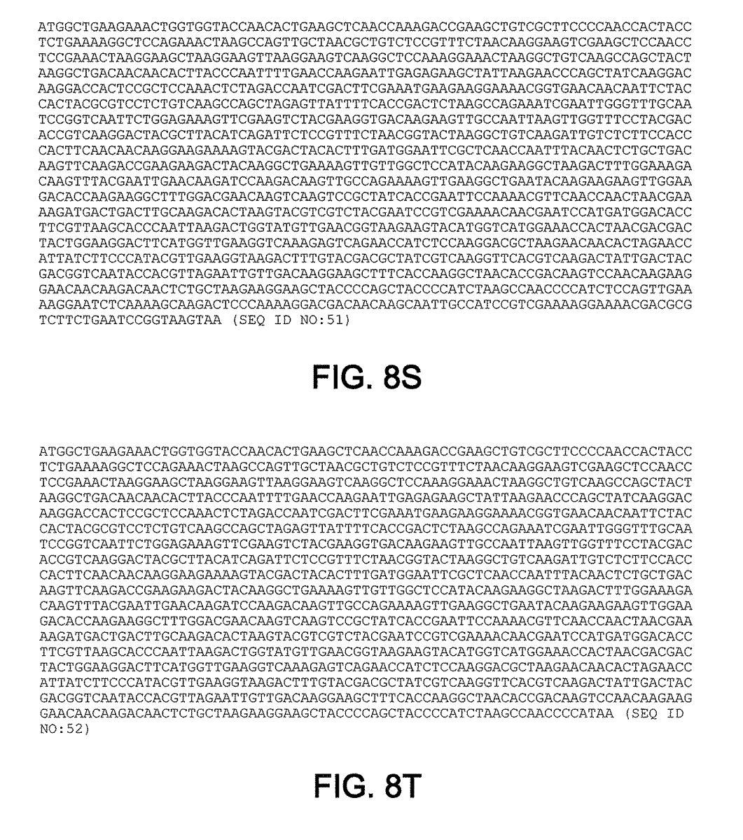

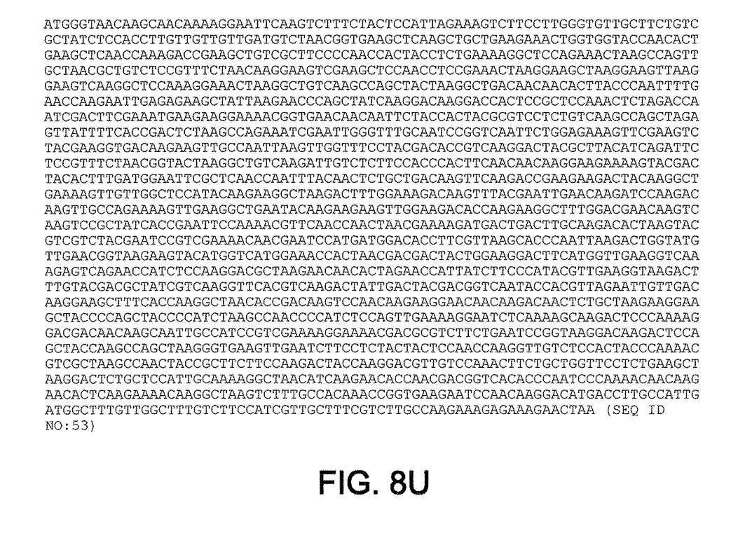

FIGS. 8C-8M provide yeast optimized nucleic acid sequences. FIG. 8C provides a yeast optimized nucleic acid sequence (SEQ ID NO: 31) encoding SEQ ID NO: 28 without a carboxyl His-Tag. FIG. 8D provides a yeast optimized nucleic acid sequence (SEQ ID NO: 32) encoding SEQ ID NO: 3. FIG. 8E provides a yeast optimized nucleic acid (SEQ ID NO: 33) sequence encoding SEQ ID NO: 1. FIGS. 8F-8M provide yeast optimized nucleic acid sequences (SEQ ID NOs: 34-41) encoding SEQ ID NO: 7 containing a methionine amino terminus. FIGS. 8N-8U provide yeast optimized nucleic acid sequences (SEQ ID NOs: 46-53) encoding different ORF0657n related polypeptides based on SEQ ID NO: 17 or SEQ ID NO: 20.

Yeast expression can be achieved using optimized sequences, and sequences not optimized for yeast expression (e.g., nucleotides 1-1935 or 124-1458 of SEQ ID NO: 29, or nucleotides 1-1710 of SEQ ID NO: 30). Techniques for yeast expression using optimized and non-optimized sequences are illustrated in the Examples infra.

ORF0657n is a surface protein containing a 36-amino acid C-terminal cell wall sorting signal with a conserved “LPXTG” motif. (Schneedwind et al. 1993, EMBO, 12: 4803-4811, 1993). Proteins containing a cell wall sorting signal are tethered to the cell wall envelope by a transpeptidation mechanism catalyzed by sortase, a membrane-bound protein (Mazmanian et al, Science 299:906-909, 2001). For tethering, the surface protein must also contain an N-terminal signal peptide for export into the secretory pathway. In the secretory pathway, the signal peptide is removed and the cell wall sorting signal facilitates retention in the secretory pathway. Sortase then cleaves between the threonine and the glycine of the LPXTG motif and catalyzes formation of an amide bond between the carboxyl-group of threonine and the amino-group of peptidoglycan cross-bridges.

Expression in yeast was found to be significantly increased by removing the cell wall sorting sequence. In different embodiments, the polypeptide encoding constructs lacks a functional cell wall sorting encoding sequence, and more preferably, lacks functional cell wall sorting and signal peptide encoding sequences. Corresponding preferred ORF0657n related polypeptides lack a functional a cell wall sorting sequence, or both the cell wall sorting and signal peptide sequences.

In different embodiments, at least substantially all of the cell wall sorting sequence or both the cell wall sorting and signal peptide sequences are not present in the polypeptide or nucleic acid encoding the polypeptide. In different embodiments, protein expression is increased at least about 10 fold, at least about 15 fold, or at least about 20 fold by removing at least substantially all of the cell wall sorting sequence or both the cell wall sorting and signal peptide sequences. More preferably, the encoding construct also contains one or more codons optimized for yeast (e.g., S. cerevisiae) expression.

Examples of approximate regions for ORF0657n cell wall sorting and signal peptide sequences can be illustrated with respect to SEQ ID NO: 2. Amino acids 1-42 contains the signal peptide sequence. Amino acids 609-645 contains the cell wall sorting sequence.

### Adjuvants

Adjuvants are substances that can assist an immunogen in producing an immune response. Adjuvants can function by different mechanisms such as one or more of the following: increasing the antigen's biologic or immunologic half-life; improving antigen delivery to antigen-presenting cells; improving antigen processing and presentation by antigen-presenting cells; and inducing production of immunomodulatory cytokines. (Vogel, Clinical Infectious Diseases 30(suppl. 3):S266-270, 2000.)

A variety of different types of adjuvants can be employed to assist in the production of an immune response. Examples of particular adjuvants include aluminum hydroxide, aluminum phosphate, or other salts of aluminum, calcium phosphate, DNA CpG motifs, monophosphoryl lipid A, cholera toxin, E. coli heat-labile toxin, pertussis toxin, muramyl dipeptide, Freund's incomplete adjuvant, MF59, SAF, immunostimulatory complexes, liposomes, biodegradable microspheres, saponins, nonionic block copolymers, muramyl peptide analogues, polyphosphazene, synthetic polynucleotides, IFN-γ, IL-2, IL-12, and ISCOMS. (Vogel Clinical Infectious Diseases 30(suppl 3):S266-270, 2000, Klein et al., Journal of Pharmaceutical Sciences 89:311-321, 2000, Rimmelzwaan et al., Vaccine 19:1180-1187, 2001, Kersten Vaccine 21:915-920, 2003, O'Hagen Curr. Drug Target Infect. Disord., 1:273-286, 2001.)

### Patients for Inducing Protective Immunity

A “patient” refers to a mammal capable of being infected with S. aureus. A patient can be treated prophylactically or therapeutically. Prophylactic treatment provides sufficient protective immunity to reduce the likelihood, or severity, of a S. aureus infection. Therapeutic treatment can be performed to reduce the severity of a S. aureus infection.

Prophylactic treatment can be performed using a vaccine containing an immunogen described herein. Such treatment is preferably performed on a human. Vaccines can be administered to the general population or to those persons at an increased risk of S. aureus infection.

Persons with an increased risk of S. aureus infection include health care workers; hospital patients; patients with a weakened immune system; patients undergoing surgery; patients receiving foreign body implants, such as a catheter or a vascular device; patients facing therapy leading to a weakened immunity; persons with burn or wound injury; and persons in professions having an increased risk of burn or wound injury. (The Staphylococci in Human Disease, Crossley and Archer (ed.), Churchill Livingstone Inc. 1997.)

Non-human patients that can be infected with S. aureus include cows, pigs, sheep, goats, rabbits, horses, dogs, cats and mice. Treatment of non-human patients is useful in protecting pets and livestock, and in evaluating the efficacy of a particular treatment.

### Anamnestic Response

ORFO657n related polypeptides were found to produce a rapid and effective immune response in Rhesus monkeys after a single dose. (See Example 17 Infra.) The observed response was consistent with an anamnestic response.

The production of an anamnestic response provides significant advantages such as the ability to provide effective immunity using a single dose and providing the effective immunity in a short period of time. In different embodiments the anamnestic response results in at least a 3-fold, at least a 5-fold, or at least a 6-fold, increase in geometric mean titres over pre-existing titers; and the fold increase is produced within 3, 5, 7, 9, 14, or 21 days.

The ability to quickly generate an effective immune response provides cost savings over multi-dose vaccinations and can be used to vaccinate a patient having an increased risk of S. aureus infection. Persons with an increased risk of S. aureus infection include health care workers; hospital patients; patients with a weakened immune system; patients undergoing surgery; patients receiving foreign body implants, such as a catheter or a vascular device; patients facing therapy leading to a weakened immunity; persons with burn or wound injury; and persons in professions having an increased risk of burn or wound injury. (The Staphylococci in Human Disease, Crossley and Archer (ed.), Churchill Livingstone Inc. 1997.) In different embodiments a patient is vaccinated immediately or within 3, 5, 7, 9, 14, or 21 days of a medical procedure.

### Combination Vaccines

ORF0657n related polypeptides providing protective immunity can be used alone, or in combination with other immunogens, to induce an immune response. Additional immunogens that may be present include: one or more additional S. aureus immunogens, such as those referenced in the Background of the Invention supra; one or more immunogens targeting one or more other Staphylococcus organisms such as S. epidermidis, S. haemolytieus, S. warneri, or S. lugunensis; and one or more immunogens targeting other infectious organisms.

### Animal Model System

An animal model system was used to evaluate the efficacy of a polypeptide to produce a protective immune response against S. aureus. Two obstacles encountered in setting up a protective animal model were: (1) very high challenge dose needed to overcome innate immunity and (2) death rate too fast to detect a protective response. Specifically, mice succumbed to infection within 24 hours after bacterial challenge which did not provide sufficient time for the specific immune responses to resolve the infection. When the dose was lowered, both control and immunized mice survived the infection.

These obstacles were addressed by using a slow kinetics lethality model involving S. aureus prepared from cells in stationary phase, appropriately titrated, and administered intravenously. This slow kinetics of death provides sufficient time for the specific immune defense to fight off the bacterial infection (e.g., 10 days rather 24 hours).

S. aureus cells in stationary phase can be obtained from cells grown on solid medium. They can also be obtained from liquid, however the results with cells grown on solid medium were more reproducible. Cells can conveniently be grown overnight on solid medium. For example, S. aureus can be grown from about 18 to 24 hours under conditions where the doubling time is about 20-30 minutes.

Staphylococcus can be isolated from solid or liquid medium using standard techniques to maintain Staphylococcus potency. Isolated Staphylococcus can be stored, for example, at −70° C. as a washed high density suspension (>10⁹ colony forming units (CFU)/mL) in phosphate buffered saline containing glycerol.

The Staphylococcus challenge should have a potency providing about 80 to 90% death in an animal model over a period of about 7 to 10 days starting on the first or second day. Titration experiments can be performed using animal models to monitor the potency of the stored Staphylococcus inoculum. The titration experiments can be performed about one to two weeks prior to an inoculation experiment.

Initial potency for titration experiments can be based on previous experiments. For the animal model strain Becker a suitable S. aureus challenge was generally found in the range of 5×10⁸ to 8×10⁸ CFU/mL.

### Administration

Immunogens can be formulated and administered to a patient using the guidance provided herein along with techniques well known in the art. Guidelines for pharmaceutical administration in general are provided in, for example, Vaccines Eds. Plotkin and Orenstein, W.B. Sanders Company, 1999; Remington's Pharmaceutical Sciences 20^{th} Edition, Ed. Gennaro, Mack Publishing, 2000; and Modern Pharmaceutics 2^{nd} Edition, Eds. Banker and Rhodes, Marcel Dekker, Inc., 1990, each of which are hereby incorporated by reference herein.

Pharmaceutically acceptable carriers facilitate storage and administration of an immunogen to a patient. Pharmaceutically acceptable carriers may contain different components such as a buffer, sterile water for injection, normal saline or phosphate buffered saline, sucrose, histidine, salts and polysorbate.

Immunogens can be administered by different routes such as subcutaneous, intramuscular, or mucosal. Subcutaneous and intramuscular administration can be performed using, for example, needles or jet-injectors.

Suitable dosing regimens are preferably determined taking into account factors well known in the art including age, weight, sex and medical condition of the patient; the route of administration; the desired effect; and the particular compound employed. The immunogen can be used in multi-dose vaccine formats. It is expected that a dose would consist of the range of 1.0 μg to 1.0 mg total polypeptide. In different embodiments of the present invention the range is 0.01 mg to 1.0 mg and 0.1 mg to 1.0 mg.

The timing of doses depends upon factors well known in the art. After the initial administration, if needed for a particular individual, one or more booster doses may subsequently be administered to maintain or boost antibody titers. An example of a dosing regime would be day 1, 1 month, a third dose at either 4, 6 or 12 months, and additional booster doses at distant times as needed.

### Generation of Antibodies

An ORF0657 related polypeptide able to induce protective immunity can be used to generate antibodies and antibody fragments that bind to the polypeptide or to S. aureus. Such antibodies and antibody fragments have different uses including use in polypeptide purification, S. aureus identification, or in therapeutic or prophylactic treatment against S. aureus infection.

Antibodies can be polyclonal or monoclonal. Techniques for producing and using antibodies are well known in the art. Examples of such techniques are described in Ausubel, Current Protocols in Molecular Biology, John Wiley, 1987-1998, Harlow et al., Antibodies, A Laboratory Manual, Cold Spring Harbor Laboratory, 1988, and Kohler et al., Nature 256:495-497, 1975.

### EXAMPLES

Examples are provided below further illustrating different features of the present invention. The examples also illustrate useful methodology for practicing the invention. These examples do not limit the claimed invention.

### Example 1

### Use of ORF0657n Region to Provide Protective Immunity

This example illustrates the ability of full-length ORF0657n region to provide protective immunity.

### ORF0657n Cloning and Expression

PCR primers were designed to amplify the gene encoding ORF0657n starting at the first asparagine residue and ending prior to the stop codon at the terminal asparagine residue. These PCR primers also had additional NcoI (forward primer) and XhoI (reverse primer) sites to facilitate cloning into the expression vector.

The encoded protein was designed to be expressed from the pET28 vector with the terminal His residues and the stop codon encoded by the vector. In addition, a glycine residue was added to the protein after the methionine initiator. The resulting amplified DNA sequence encodes a carboxyl His-Tag ORF0657n (SEQ ID NO: 28).

PCR amplified sequences were ligated into the pET28 vector (Novagen) using the NcoI/XhoI sites that had been engineered into the PCR primers and introduced into E. coli DH5α (Invitrogen) by heat shock. The transformation mixture was grown overnight on Luria-Bertani (LB) agar plates with 100 μg/mL kanamycin at 37° C. Colonies were selected, grown in LB with 30 μg/mL kanamycin, DNA minipreps made (Promega), and insert integrity determined by restriction digestion and PCR. Four minipreps with correct insert size were sequenced using the primers M13F (SEQ ID NO: 65), M13R (SEQ ID NO: 66), ORF0657 nF (SEQ ID NO: 67), and ORF0657nR (SEQ ID NO: 68). A clone was selected containing no DNA changes from the desired sequence.

E. coli HMS174(DE3) cells (Novagen) were transformed and grown on LB plates containing kanamycin (30 μg/mL); 3 colonies (UnkC-1, UnkC-2 and UnkC-3) were selected for expression testing. Liquid LB (kanamycin) cultures were incubated at 37° C., 250 rpm until the A₆₀₀ was between 0.6 and 1.0 and then induced by the addition of IPTG to a final concentration of 1 mM followed by three hours further incubation. Cultures were harvested by centrifugation at 5000×g for 5 minutes at 4° C. Cells were resuspended in 500 μL lysis buffer (Bug Buster, with protease inhibitors, Novagen). An equal volume of loading buffer (supplemented with β-mecapto ethanol to 5% final volume) was added prior to heating the samples at 70° C. for 5 minutes. Extracts were run on Novex 4-20% Tris-Glycine gels and proteins were visualized (Coomassie Blue stained) and blotted onto nitrocellulose and probed with anti-HIS6 antibodies (Zymed).

### ORF0657n Purification

Direct scale-up of the above small scale procedure into stirred tank fermenters (75 liter scale) with a 50 liter working volume was achieved. Inoculum was cultivated in a 250 mL flask containing 50 mL of Luria-Bertani (LB) medium (plus kanamycin) and inoculated with 1 mL of frozen seed culture and cultivated for 3 hours. One mL of this seed was used to inoculate a 2 liter flask containing 500 mL of LB medium (plus kanamycin) and incubated for 16 hours. A large scale fermenter (75 liter scale) was cultivated with 50 liters of LB medium (plus kanamycin). The fermentation parameters of the fermenter were: pressure=5 psig, agitation speed=300 rpms, airflow=15 liters/minute and temperature=37° C. Cells were incubated to an optical density (OD) of 0.8 optical density units, at a wavelength of 600 nm, and induced with Isopropyl-β-K-Thiogalactoside (IPTG) at a concentration of 1 mM. Induction time, with IPTG, was three hours. Cells were harvested by lowering the temperature to 15° C., concentrated by passage through a 500KMWCO hollow fiber cartridge, and centrifuged at 9,000 times gravity at 4° C. for 20 minutes. Supernatants were decanted and the recombinant E. coli wet cell pellets were frozen at −70° C.

Recombinant E. coli cells (19.2 g wet cell weight) were suspended in Lysis Buffer (50 mM Tris-HCl, pH 8.0, 0.1 M NaCl, 2 mM MgCl₂, 10 mM imidazole, 0.1% Tween™-80, and 6 M guanidine-HCl) at 8 mL per gram of cell wet weight. Protease Inhibitor Cocktail for use with poly-(Histidine)-tagged proteins (Sigma, P8849) was added to the suspension at 0.05 mL per gram of cell paste. Additionally, Lysozyme was added to 1 mg/mL, and Benzonase™ (EM Ind.) was added to 1 μL/mL. Cell lysis was accomplished by passing the suspension through a microfluidizer at 14,000 PSI (Microfluidics Model 110S) four times at 4° C. Cell debris was pelleted at 11,000×g for 30 minutes at 4° C., and the supernatant retained.

Proteins bearing a His-Tag were purified from the supernatant. The supernatant was mixed with 20 mL of Ni⁺-NTA agarose (Qiagen) at 4° C. with gentle inversion for 2 hours. The mixture was poured into an open column (1.5 cm×20 cm) and the non-bound fraction was collected in bulk. The column was washed with Wash Buffer (20 mM Tris-HCl, pH 8.0, 0.15 M NaCl, 0.1% Tween™-80). His-Tagged ORF0657n was eluted with a step gradient of 300 mM imidazole, 20 mM Tris-HCl, pH 7.5, 0.15 M NaCl, 0.1% Tween™-80.

Fractions containing His-Tag ORF0657n (SEQ ID NO: 28) were detected by Coomassie stained SDS-PAGE and pooled. Pooled fractions were filtered through a 0.2 micron filter to remove particulate material, and were applied on a size-exclusion column (Sephacryl S-300 26/60 column, Amersham Biosciences) and eluted at 1 mL/min with 10 mM MOPS pH 7.1, 150 mM NaCl. Fractions containing His-Tag ORF0657n were detected by Coomassie stained SDS-PAGE and Western blotting (anti-tetra His Mab, Qiagen). Endotoxin was removed by filtration through a Zeta-Plus™ Biofilter (CUNO). Protein concentration was determined by BCA (Pierce). Purity was determined by densitometry of Coomassie stained gels.

### Example 2

### Preparation of S. aureus Challenge

S. aureus was grown on Tryptic Soy Agar (TSA) (Becton Dickinson, Sparks, Md.) plates at 37° C. overnight. The bacteria were washed from the ISA plates by adding 5 mL of PBS onto a plate and gently resuspending the bacteria with a sterile spreader. The bacterial suspension was spun at 6000 rpm for 20 minutes using a Sorvall RC-5B centrifuge (DuPont Instruments). The pellet was resuspended in 16% glycerol and aliquots were stored frozen at −70° C.

Prior to use, inocula were thawed, appropriately diluted and used for infection. Each stock was titrated at least 3 times to determine the dose appropriate for inducing slow kinetics of death in naive mice. The potency of the bacterial inoculum (ability to kill 80 to 90% of the mice) was constantly monitored to assure reproducibility of the model. Ten days before each challenge experiment, a group of 10 control animals (immunized with adjuvant alone) were challenged and monitored.

### Example 3

### Protection Studies Using His-Tagged ORF0657n Related Polypeptides

Twenty-five BALB/c mice were immunized with three doses of His-Tag ORF0657n (SEQ ID NO: 28), 20 μg per dose, on aluminum hydroxyphosphate adjuvant (450 μg per dose). Aluminum hydroxyphosphate adjuvant (AHP) is described by Klein et al., Journal of Pharmaceutical Sciences 89, 311-321, 2000. The doses were administered as two 50 μL intramuscular injections on days 0, 7 and 21. The mice were bled on day 28, and their sera were screened by ELISA for reactivity to His-Tag ORF0657n.

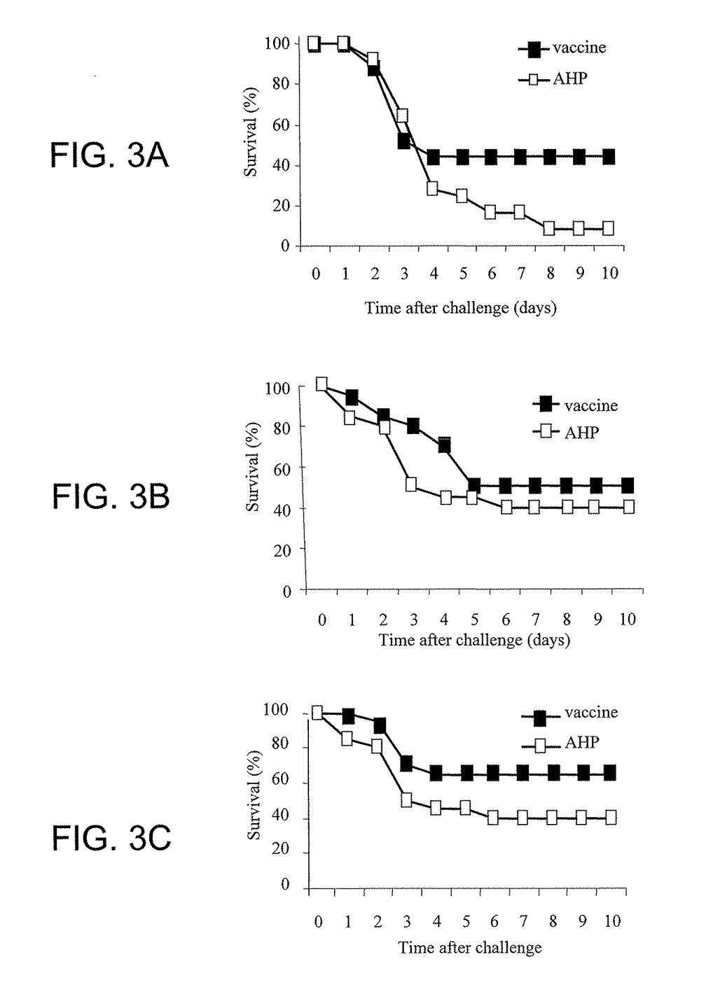

On day 35 of the experiment the mice were challenged by intravenous injection of S. aureus grown, at a dose (7.3×10⁸ CFU/mL) determined in titration experiments to cause death over a period of approximately 2 to 7 days. Survival in this lethal model with slow kinetics of death was evaluated against a control set of mice that had just been sham-immunized with AHP. The mice were monitored over a 14 day period for survival (FIG. 3A). At the end of the experiment 11 mice survived the ORF0657n immunized group compared to three surviving in the AHP control group.

FIGS. 3B and 3C illustrate protection using polypeptides corresponding to the ORF0657nH and ORF0657nI regions. FIG. 3B illustrates protection with SEQ ID NO: 4 containing a carboxyl His-Tag. FIG. 3C illustrates results with SEQ ID NO: 5 containing a carboxyl His-Tag.

### Example 4

### Obtaining ORF0657n Sequences

ORF0657n has been implicated to have a role in S. aureus iron acquisition. (Andrade et al., Genome Biology 3(9):47.1-47.5, 2003.) ORF0657n sequences, some of which are from different sources, have been given different designations in different references. (For example, see, Etz et al., PNAS USA, 99:6573-6578, 2002 (LPXTGVI); Baba et al., The Lancet 359:1819-1827, 2002 (MW1011); Kuroda, et al., The Lancet 357, 1225-1240, 2001 (SA0976); Andrade et al., Genome Biology 3(9):47.1-47.5, 2003 (S_aur2); Mazmanian et al., Science 299:906-909, 2003 (isdB); Mazmanian et al., Molecular Microbiology 40:1049-1057, 2001 (sasJ); and Taylor et al., Mol. Microbiol. 43:1603-1614, 2002 (sirH).

A polypeptide sequence corresponding to a ORF0657n protein sequence appears to be provided in different patent publications. (Meinke et al., International Publication Number WO 02/059148, published Aug. 1, 2002, Wang et al., International Publication Number WO 02/077183, published Oct. 3, 2002, Masignani et al., International Publication Number WO 02/094868, published Nov. 28, 2002, Foster et al., International Publication Number WO 02/102829, published Dec. 27, 2002, and Foster et al., International Publication Number WO 03/011899, published Feb. 13, 2003.)

Genomic DNA was obtained from different S. aureus clinical isolates. Clinical isolates were added to 3 mL of Difco Tryptic Soy Broth (Becton Dickinson, Sparks, Md.) and incubated overnight at 37° C. and 150 rpm. The overnight cultures were centrifuged in 1.5 mL Eppendorf tubes at 14,000 rpm for 5 minutes. The broth was decanted and the pellets re-suspended in 500 μL, re-suspension buffer (25% sucrose, 10 mM Tris pH 7.5). A 5 μL aliquot of a 2 mg/ml lysostaphin (Sigma-Aldrich, St. Louis, Mo.) solution was added to each resuspended pellet. Suspensions were then incubated at 37° C. for 1 hour.

At the end of the incubation period, 250 μL of 2% SDS was added to each tube and vortexed until the viscosity of the solution noticeably decreased. 250 μL phenol-chloroform-isoamyl solution (25:24:1, v/v) (Gibco/Invitrogen Corporation, Grand Island, N.Y.) were added. The mixture was vortexed for 30 seconds and centrifuged for 5 minutes at 14,000 rpm. The top aqueous phase was removed and the precipitation steps were repeated until barely any interface remained. 0.1 volume of 3 M NaOAc, pH 4.8, was added to each tube and mixed. One volume of isopropanol was then added and mixed again. The tubes were left to incubate 5 minutes at room temperature and then centrifuged at 14,000 rpm for 15 minutes. The supernatant was decanted and tubes were allowed to dry upside-down on tissue. The pellets were resuspended in 50 μL, sterile H₂O.

The isolated DNA was used as a template for PCR. The gene was amplified using a forward primer (ORF0657 nF, SEQ ID NO: 67) and reverse primer (ORF0657nR, SEQ ID NO: 68). PCR products were sequenced using standard Big Dye protocols.

### Example 5

### Comparison of ORF0657n from Different S. aureus Isolates

ORF0657n was found to be well conserved across a collection of pathologically and taxonomically diverse S. aureus clinical isolates. Table 3 provides a summary of the percent identity among different isolates including clinical isolates.

TABLE 3

| | | | | | | ORF0657n |
|---|---|---|---|---|---|---|
| Strain | MLST | Source | Country | | CP | % ID |
| CL-1 | 1 | Hyper-virulent community blood | UK | MSSA | 8 | 99 |
| | | isolate | | | | |
| MW2 | 1 | Hyper-virulent community blood | USA | MRSA | 8 | 99 |
| | | isolate | | | | |
| CL-2 | 1 | Left forearm wound | USA | MSSA | 8 | 99 |
| CL-3 | 1 | Arm wound | USA | MSSA | 8 | 99 |
| CL-4 | 5 | Left foot | USA | MRSA | 5 | 99 |
| CL-5 | 5 | Wound | USA | MRSA | 5 | 100 |
| Mu 50 | 5 | Pus, Surgical wound infection | Japan | VISA | 5 | 100 |
| N315 | 5 | Pharyngeal smear | Japan | MRSA | 5 | 100 |
| CL-6 | 5 | CDC3 | USA | MRSA/ | 5 | 97 |
| | | | | VISA | | |
| CL-7 | 8 | Blood | Germany | MRSA | 5 | 98 |
| NCTC8325 | 8 | MSSA lab strain | USA | MSSA | 5 | 99 |
| CL-8 | 8 | Right leg wound | USA | MRSA | 5 | 99 |
| CL-90 | 9 | Nares | UK | MSSA | 5 | 97 |
| CL-10 | 9 | Blood | UK | MSSA | 5 | 97 |
| CL-11 | 12 | Blood | NL | MSSA | 8 | 97 |
| CL-12 | 12 | Nares | UK | MSSA | 8 | 97 |
| CL-13 | 15 | Left finger skin | USA | MSSA | 8 | 99 |
| CL-14 | 15 | Pus left anaresle | USA | MSSA | 8 | 99 |
| CL-15 | 15 | Blood | UK | MSSA | 8 | 99 |
| CL-16 | 22 | Blood | Canada | MSSA | 5 | 99 |
| CL-17 | 22 | Barnin MRSA | Germany | MRSA | 5 | 98 |
| CL-18 | 25 | Blood | Canada | MRSA | 5 | 97 |
| CL-19 | 25 | Blood | UK | MSSA | 5 | 97 |
| CL-20 | 25 | Nares | UK | MSSA | 5 | 97 |
| CL-21 | 30 | Source not known mecIV | Sweden | MRSA | 8 | 94 |
| CL-22 | 30 | Source not known mecIV | Germany | MRSA | 8 | 94 |
| CL-23 | 36 | Epidemic MRSA blood | UK | MRSA | 8 | 94 |
| CL-24 | 45 | Left leg stump pus | USA | MRSA | 8 | 95 |
| CL-25 | 45 | Nares | USA | MRSA | 8 | 95 |
| Becker | 45 | Prototype capsule 8 strain | USA | MSSA | 8 | 95 |
| COL | 88 | MRSA lab strain | USA | MRSA | 5 | 100 |
| CL-26 | 97 | Blood | NL | MSSA | 5 | 98 |
| CL-27 | 97 | Blood | Canada | MSSA | 5 | 99 |
| CL-28 | 97 | Nares | UK | MSSA | 5 | 99 |
| CL-29 | 121 | Nares, community disease | UK | MSSA | 8 | 96 |
| CL-30 | 121 | Blood | UK | MSSA | 8 | 96 |

CP, capsule type;
MLST, Multilocus sequence typing taxonomic groupings;
MRSA methicillin resistant S. aureus;
MSSA, methicillin sensitive S. aureus
“CL” indicates clinical isolate.
“ID” indicates identity.
“VISA” indicates vancomycin intermediate S. aureus.

Percent identity (% ID) was determined by aligning the polypeptide sequence with SEQ ID NO: 2 and determining the number of identical amino acids. This number was divided by the total number of amino acids (of SEQ ID NO: 2) and then multiplied by 100 and rounded to the nearest whole number.

### Example 6

### Protection Against Different S. aureus Clinical Isolates

The efficacy of ORF0657n as immunogen against different S. aureus clinical isolates was evaluated using His-Tag ORF0657n as an immunogen (SEQ ID NO: 28). A subset of taxonomically diverse isolates described in Table 3 were used as challenge inocula. The ORF0657n sequences in these different isolates differed from the employed vaccine OFR0657n sequence.

The challenge strains obtained were prepared using the techniques described in Example 2. Mice were immunized and challenged using the techniques described in Example 3, and monitored for 10 days. The challenge inocula routinely used in the models far exceeds those typically encountered in human infections.

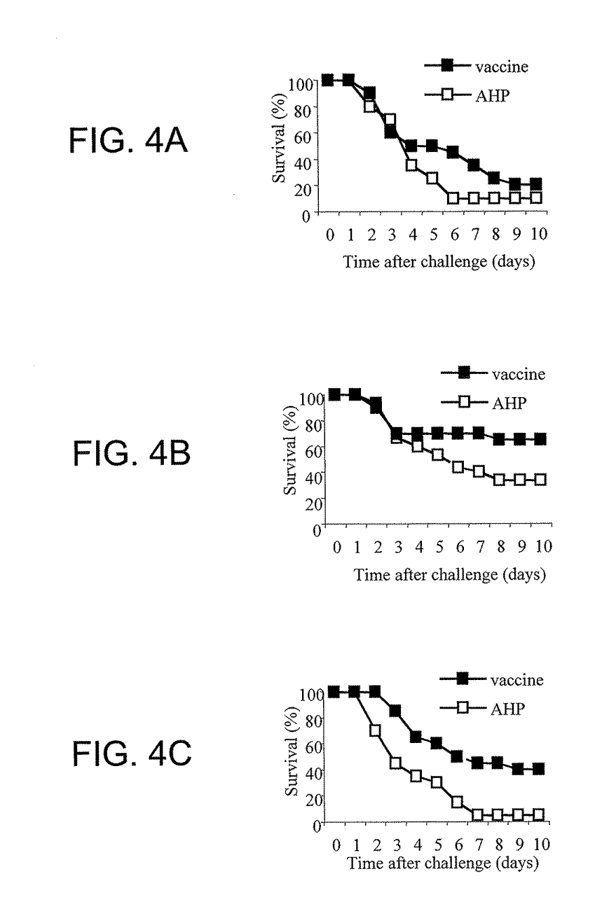

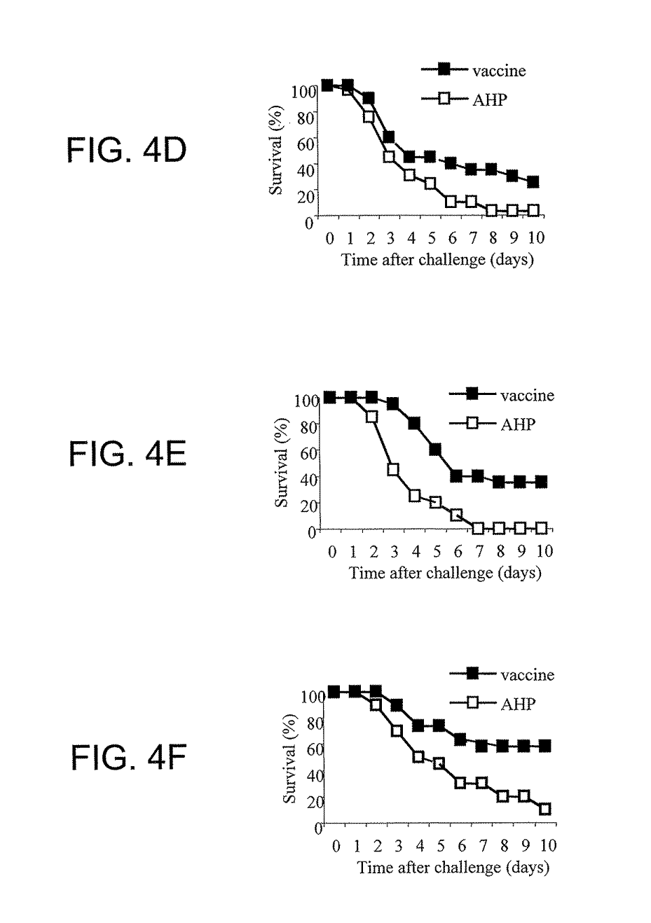

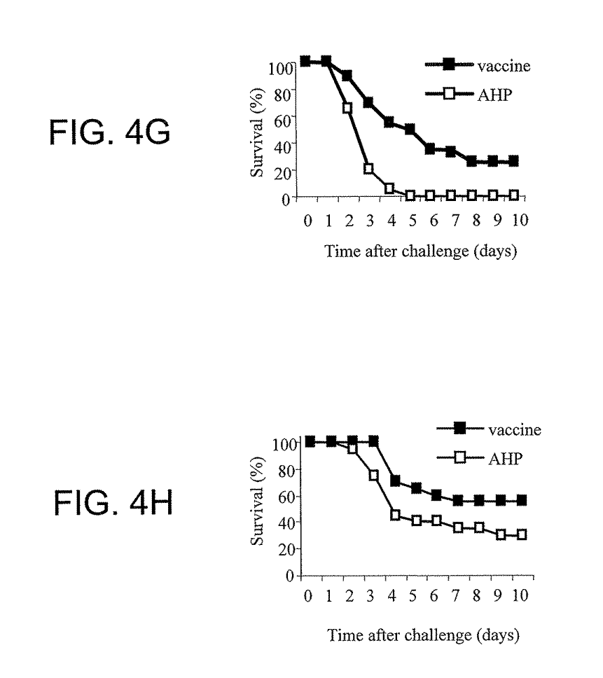

Protection was demonstrated against both methicillin sensitive and resistant strains. The results are shown in FIGS. 4A-4H.

### Example 7

### Codon Optimization of SEQ ID NO: 28 for Yeast Expression

The nucleic acid sequence encoding amino acids 1-646 of SEQ ID NO: 28 was codon optimized for expression in yeast. A codon-optimized sequence for amino acids 1-646 of SEQ ID NO: 28 is shown in FIG. 8C (SEQ ID NO: 31).

SEQ ID NO: 29 without the carboxyl His-Tag encoding region was used as the starting construct for optimization. The overall codon usage of the encoding sequence prior to optimization was: 28% of the codons (179) were very rare or never used by highly expressed yeast genes and 20% (126) were moderately rare.

Codon optimization of nucleic acid encoding amino acids 1-646 of SEQ ID NO: 28 was performed to replace codons having a low or moderate expression with codons highly expressed in yeast. In addition, a glycine codon was added to the second position. Codon optimization was performed using the software program MacDNAsis Pro V3.0. The employed parameter table used for backtranslating proteins indicates highly expressed S. cerevisiae codons. In MacDNAsis Pro, the function used is “Translate>[Protein->DNA]”. The output is entitled “Amino Acid Conversion.”

In some cases, there is more than one highly expressed codon for a given amino acid. For example, serine is encoded by either “TCT” or “TCC”. In these cases, approximately equal numbers of the two different codons were employed. Table 4 provides a codon table for highly expressed S. cerevisiae codons.

TABLE 4

| Amino | Optimal |
|---|---|
| Acid | Codon(s) |
| Phe | TTC |
| Leu | TTG |
| Ile | ATT, ATC |
| Met | ATG |
| Val | GTT, GTC |
| Ser | TCT, TCC |
| Pro | CCA |
| Thr | ACT, ACC |
| Ala | GCT |
| Tyr | TAC |
| His | CAC |
| Gln | CAA |
| Asn | AAC |
| Lys | AAG |
| Asp | GAC |
| Glu | GAA |
| Cys | TGT |
| Trp | TGG |
| Arg | AGA |
| Gly | GGT |

All of the codons that were not optimal were changed to codons found in highly expressed yeast genes with the exception of two alanine codons that were changed to codons found in moderately expressed genes (GCG beginning at nucleotides 505 and 1546) of SEQ ID NO: 31.

The overall sequence encoding ORF0657n was prepared by the annealing and extension of 25 oligomers (SEQ ID NOs: 69-93) designed to encode the final desired sequence. The oligomers were 85-110 bp in length. The oligomers were alternating, ORF0657n coding sequence. Each oligomer had a complementary overlap of 25-29 by with the adjoining oligomer and the duplex had a Tm of 80-84° C. This was calculated manually by assigning a GC base-pair a value of 4° C., and an AT base-pair as 2° C.

Seven separate extension reactions were performed using three or four adjoining overlapping oligomers and sense and antisense PCR primers (23-26) nt in length with duplex Tm=70-72° C. Native Pfu DNA polymerase (STRATAGENE, La Jolla, Calif.) was used for the PCR reactions in a “touch down” strategy as follows: 95° C., 90 seconds, one cycle; 95° C., 30 seconds, 55° C., 30 seconds, 68° C., 3 minutes for 5 cycles, immediately followed by a second series of reactions; 95° C., 30 seconds, 52° C., 30 seconds, 68° C., 3 minutes for 20 cycles. The reaction was completed by incubation at 68° C. for 7 minutes. As a result of these PCR reactions, seven colinear fragments of the gene were created (referred to herein for convenience as 1, 2, 3, 4, 5, 6, and 7).

The fragments were isolated by agarose gel electrophoresis and products of the appropriate size were excised and purified using the GENE CLEAN® II method (QBIOgene, Carlsbad, Calif.) as recommended by the manufacturer. Colinear fragments 1, 2, and 3, colinear fragments 4 and 5, and colinear fragments 6 and 7 were combined in subsequent PCR reactions with the appropriate primers to yield fragments A, B, and C, respectively. The complete gene for ORF0657n was then assembled by an additional PCR reaction in which fragments A, B, and C were combined using distal sense and antisense primers. The final PCR product was gel-isolated and cloned into pCR®-Blunt II-TOPO® (INVITROGEN, Carlsbad, Calif.) as recommended by the manufacturer. The DNA sequence of several independent clones was obtained and errors identified.

Errors were corrected on different segments of three independent clones, pUC3, pUC4, and pUC6, either sequentially using the QUIK-CHANGE Site-directed Mutagenesis Kit, or simultaneously, with the QUIK-CHANGE Site-directed Multi Mutagenesis Kit (STRATAGENE, La Jolla, Calif.) according to the manufacturer's recommendations. The final corrected sequence was obtained by swapping repaired restriction fragments from the three clones. A repaired 1.1-kb XmnI fragment of clone pUC4 was swapped for the corresponding XmnI fragment of pUC3 to construct pUnkC13. A repaired 456-bp AccI fragment of pUC6 was swapped for the corresponding fragment of pUnkC13 to construct pUnkCR1 and the DNA sequence was verified.

### Example 8

### Construction of Saccharomyces cerevisiae Strains for Recombinant Gene Expression

This example illustrates techniques that can be employed to obtain Saccharomyces cerevisiae strains for recombinant gene expression. The creation of strains designated 1260 and 1309 is described below. As the genetic background of a strain can greatly influence the properties of a strain for heterologous protein expression, it was desired to construct yeast strains with differing genetic backgrounds which also contained several desirable genetic markers: (1) mnn9 mutation to prevent hyperglycosylation of secreted proteins, (2) prb1 and/or pep4 protease mutations to reduce problems with proteolysis, and (3) overexpression of the GAL4 transcription factor in order to increase expression from GAL promoters.

Construction of S. cerevisiae Strain Designated 1260

A starting S. cerevisiae strain was constructed in the following manner. The S. cerevisiae strain Y379-5D (MATα, cyh2, nib1, rho⁻, cir°) (Livingston, Genetics 86:73-84, 1977) was crossed with strain DC04 (MATa, ade1, adeX, leu2-04 cir°) (Broach et al., Cell 21:501-508, 1980). The resulting diploid strain was sporulated and tetrads were dissected by standard procedures. One of the haploid spores gave rise to the strain 2150-2-3 (MATa, ade1, leu2-04, cir°). The α-mating type S. cerevisiae strain LB-347-1C (MATα, mnn9) is S. cerevisiae strain X2180-1B (MATα, SUC2, mal, mel, gal2, CUP1; ATCC Number 204504) containing a mnn9 mutation. LB-347-1C was mated with the type strain 2150-2-3 (MATa, leu2-04, ade1) by mixing the strain on a YEHD complete media agar plate (Carty et al., J. Ind. Micro 2:117-121, 1987). To select for diploids, the mated strains were replica-plated onto minimal media without leucine and containing 2% sucrose as the sole carbon source. After isolating single colonies, the diploids were sporulated, and asci were dissected by standard techniques. The KHY-107 strain was isolated as a single haploid spore and characterized as ADE1, leu2 and mnn9 (by Schiff stain technique). A frozen stock in glycerol was established and stored at −70° C.

KHY-107 (cir⁺) was grown from the −70° C. stock and transformed with the yeast vector pC1/1, which contains the yeast LEU2-d gene and is related to pJDB219 (Beggs, Nature 275:104-109, 1978), except that the pMB9 DNA is replaced by pBR322 DNA. An isolated transformant was grown on selective liquid medium (lacking leucine and containing 1 M sorbitol), then grown through multiple passages in rich medium (containing 1 M sorbitol) to lose both pC1/1 and 2μ DNA. Colonies that had lost pC1/1 (unable to grow on medium lacking leucine) were checked by DNA-DNA hybridization for the presence of 2μ DNA. An isolated clone, KHY-107(cir°)-1, showing no 2μ DNA, was chosen and established as a frozen stock (−70° C.) in glycerol.

KHY-107(cir°)-1 was made ura3 by gene disruption. A plasmid was constructed containing the S. cerevisiae URA3 gene disrupted by 2 copies of the Aph3′I gene from Tn903. The 5′-URA3-Aph3′I-URA3-3′ cassette was excised from the vector and used to transform KHY-107(cir°)-1. Transformants that had integrated the disrupted ura3 cassette were selected on 5-fluoro-orotic acid plates (Kaiser, C. et al., Methods in Yeast Genetics—1994 Edition, Cold Spring Harbor Laboratory Press, (Cold Spring Harbor, N.Y.; 1994) pages 214-215), and subsequently shown to be unable to grow on medium lacking uracil. One isolated clone, KHY-107ura3(PN₂), was chosen and established as a frozen (−70° C.) stock in glycerol.

KHY-107ura3(PN₂) was grown on complex medium (containing 1 M sorbitol), then plated onto synthetic medium containing canavanine instead of arginine and canavanine-resistant (can^{R}) mutants were obtained. Spontaneous can^{R} mutants were streaked for isolated colonies on solid synthetic medium containing canavanine instead of arginine. Isolated colonies were shown by standard genetic complementation tests to be can1. One isolated can1 colony, DMY10, was chosen and preserved as a frozen (−70° C.) stock in glycerol.

To overexpress the yeast GAL4 transcription factor, the GAL10p-GAL4 fusion gene was integrated into the HIS3 gene of DMY10. The 5′-HIS3-GAL10p-GAL4-URA3-HIS3-3′ cassette was excised from pKHint-C (Schultz et al., Gene 61:123-133, 1987) and used to transform DMY10. Transformants that had integrated the cassette were selected on solid synthetic medium lacking uracil, and subsequently shown to be unable to grow on medium lacking histidine. The integration of the cassette only at the HIS3 locus was verified by Southern hybridization. One isolated integrant, DMY10int-3, was chosen and established as a frozen (−70° C.) stock in glycerol. This strain was designated CF52.

Plasmid FP8ΔH bearing the S. cerevisiae PRB1 gene (Moehle et al., Genetics 15:255-263, 1987) was digested with HindIII plus XhoI and the 3.2-kbp DNA fragment bearing the PRB1 gene was gel-purified and made blunt-ended by treatment with T4 DNA polymerase. The plasmid pUC18 was digested with BamHI, gel-purified and made blunt-ended by treatment with T4 DNA polymerase. The resulting vector fragment was ligated with the above PRB1 gene fragment to yield the plasmid pUC18-PRB1. Plasmid YEp6 (Struhl et al., Proc. Natl. Acad. Sci., USA 76:1035, 1979) which contains the HIS3 gene, was digested with BamHI. The resulting 1.7-kbp BamHI fragment bearing the functional HIS3 gene was gel-purified and then made blunt-ended by treatment with T4 DNA polymerase. The pUC18-PRB1 vector was digested with EcoRV plus NcoI which cut within the PRB1 coding sequence and removes the protease B active site and flanking sequence. The 5.7-kbp EcoRV-NcoI fragment bearing the residual 5′ and 3′-portions of the PRB1 coding sequence in pUC18 was gel-purified, made blunt-ended by treatment with T4 DNA polymerase, dephosphorylated with alkaline phosphatase, and ligated with the blunt-ended HIS3 fragment described above. The resulting plasmid (designated pUC18-prb1::HIS3, stock #1245) contains the functional HIS3 gene in place of the portion of the PRB1 gene which had been deleted above.

The PRB1 gene disruption vector (pUC18-prb1::HIS3) was digested with SacI plus XbaI to generate a linear prb1: HIS3 disruption cassette and used for transformation of strain CF52 by the lithium acetate method (Ito et al., J Bacteriol. 153:163, 1983). His⁺ transformants were selected on synthetic agar medium lacking histidine and restreaked on the same medium for clonal isolates. Genomic DNA was prepared from a number of the resulting His⁺ isolates, digested with EcoRI and then electrophoresed on 0.8% agarose gels. Southern blot analyses confirmed the presence of the desired prb1Δ::HIS3 gene disruption.

One of the isolates containing the desired prb1Δ::HIS3 disruption was selected for further use and was designated strain #1260. Frozen stocks in glycerol were prepared for strain #1260 for storage at −70° C. The resulting genotype of strain 1260 is as follows: MATa, leu2-2,112, mnn9, ura3Δ, can1, his3Δ::GAL10p-GAL4-URA3, prb1Δ::HIS3, cir°.

Construction of S. cerevisiae Strain Designated 1309

The S. cerevisiae strain BJ1995 (MATα, leu2, trp1, ura3-52, prb1-1122, pep4-3, gal2) has been described previously (Jones, E. W., Tackling the Protease Problem in Saccharomyces cerevisiae, Methods in Enzymology 194 (1991), pages 428-453). A cir° derivative of BJ1995 which lacked the endogenous 2μ DNA plasmid was isolated using the procedure disclosed for the construction of strain 1260. The resulting cir° isolate was designated strain 91 and was preserved as a frozen stock (−70° C.) in glycerol.

A derivative of strain 91 was then constructed which overproduces the GAL4 transcription factor in order to increase transcription from GAL promoters. The plasmid pKHint-C (Schultz et al., Gene 61:123-133, 1987) was digested with BamHI and the resulting 5′-HIS3-GAL10p-GAL4-URA3-HIS3-3′ cassette was used to transform strain 91. Transformants that had integrated the cassette were selected on solid synthetic medium lacking uracil, and subsequently shown to be unable to grow on medium lacking histidine. The desired integration of the cassette at the HIS3 locus was verified by Southern hybridization using a probe for the yeast HIS3 gene. One isolated integrant, BJ1995cir°int #22, was chosen and established as a frozen (−70° C.) stock in glycerol. This isolate was designated strain 1282.

A derivative of strain 1282 containing a disruption of the MNN9 gene was then isolated in the following series of steps. In order to disrupt the MNN9 gene, it was necessary to first clone the MNN9 gene from S. cerevisiae genomic DNA for the purpose of preparing a gene disruption vector. This was accomplished by standard PCR technology. A 5′ sense primer and 3′ antisense primer for PCR of the full-length MNN9 coding sequence were designed based on the published sequence for the yeast MNN9 gene (MacKay et al., European Publication Number EP0314096, International Publication Date May 3, 1989). The following oligodeoxynucleotide primers containing flanking HindIII sites (underlined) were used:

sense primer (SEQ ID NO: 94): 5′-CTT AAA GCT TAT GTC ACT TTC TCT TGT ATC G-3′
antisense primer (SEQ ID NO: 95): 5′-TGA TAA GCT TGC TCA ATG GTT CTC TTC CTC-3′ The initiating methionine codon for the MNN9 gene is highlighted in bold print.

PCR was conducted using genomic DNA from S. cerevisiae as a template. The resulting 1.2-kbp PCR fragment carrying the MNN9 gene was digested with HindIII, gel-purified, and ligated with HindIII-digested, alkaline-phosphatase treated pUC13. The resulting plasmid was designated p1183. The yeast TRP1 gene was isolated from YRp7 (Struhl et al., Proc. Natl. Acad. Sci. USA 76:1035-1039, 1979) as a 0.85-kb EcoRI-BglII fragment which was made flush-ended and then inserted into the PmlI site of p1183. The PmlI site is in the middle of the MNN9 coding sequence, thereby insertion of the TRP1 gene at that site results in a gene disruption. The resulting plasmid pUC13-mnn9::TRP1 (p11885-239-1) was then digested with HpaI plus EcoRI and the 5′-mnn9-TRP1-mnn9-3′ cassette was used for transformation of strain 1282 by the lithium acetate method. (Ito et al., J. Bacteriol. 153:163, 1983.)

Trp⁺ transformants were selected on synthetic medium agar plates lacking tryptophan and restreaked on the same plates for single colony isolates. Genomic DNA was prepared from a number of the resulting Trp⁺ isolates, digested with Hindi III, and then electrophoresed on 0.8% agarose gels. Southern blot analyses confirmed the presence of the desired mnn9::TRP1 gene disruption. One of these isolates containing the desired mnn9::TRP1 disruption was selected for further use and was designated strain 1309. Frozen stocks in glycerol were prepared for strain 1309 for storage at −70° C. The genotype for strain 1309 is as follows: MATα, leu2, mnn9::TRP1, trp1, ura3-52, his3Δ::GAL10p-GAL4-URA3, prb1-1122, pep4-3, gal2, cir°.

### Example 9

### Expression of Full-Length ORF0657n Region

Full-length ORF0657n was expressed in E. coli (SEQ ID NO: 28; His-Tag ORF0657n) and S. cerevisiae (amino acids 1-646 of SEQ ID NO: 28) and the expression products were compared.

S. cerevisiae Expression

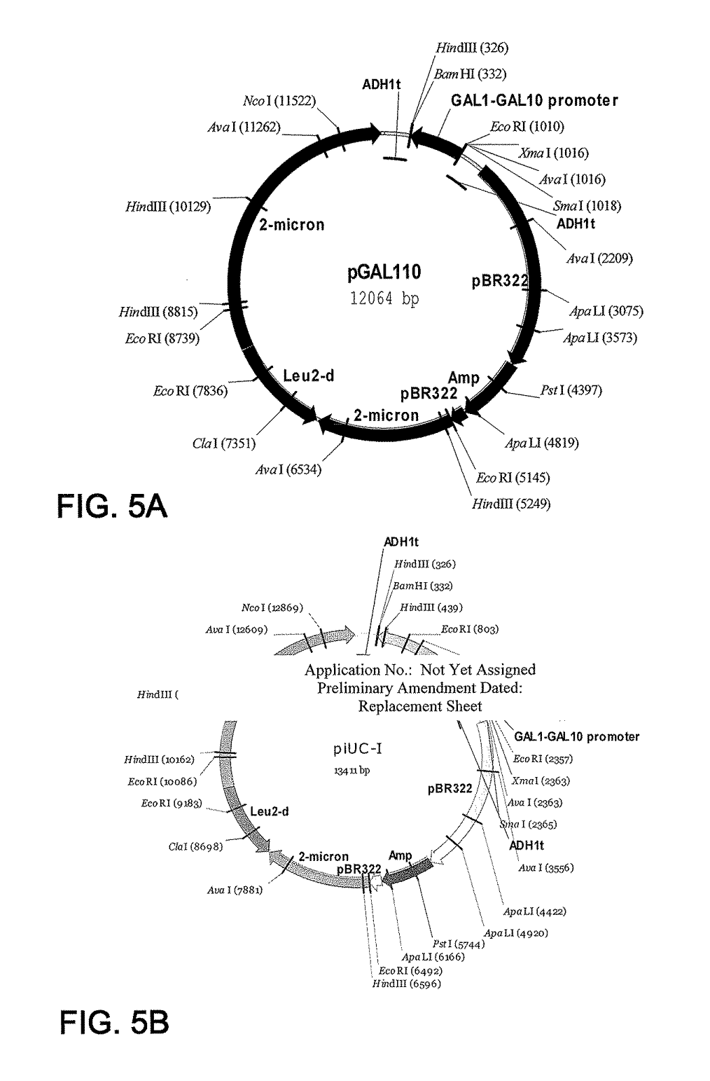

DNA encoding the ORF0657n protein with codons optimized for yeast expression was amplified from the final corrected clone, pUnkCR1 (Example 7), using the following sense and antisense primers, respectively, UnkCY-F (SEQ ID NO; 96), 5′AAC CGG TTT GGA TCC CAC AAA ACA AAA TGG GTA ACA AGC AAC AAA AGG AAT TC3′ and UnkCY-R (SEQ ID NO: 97), 5′AAC CGG TTT GGA TCC TTA GTT CTT TCT CTT TCT TGG CAA GAC3′ containing flanking BamHI restriction sites. The sense primer contains a 5′ untranslated sequence and ATG codon and the antisense primer contains TAA as the stop codon. The resulting 1.9-kb product was gel-isolated and cloned into the TOPO TA vector pCR2.1 (INVITROGEN CORPORATION, Carlsbad, Calif.) according to the manufacturer to construct plasmid UnkC-B1. The DNA sequence of the insert was subsequently verified. The BamHI fragment was gel-isolated and subcloned in the proper orientation into a yeast vector (pGAL110, FIG. 5A) to construct pRUnkC-pGAL110. The sequence of the insert was verified by DNA sequencing.

Plasmid DNA from the pRUnkC-pGAL110 was used to transform S. cerevisiae strains containing a leu2 mutation to leucine prototrophy (Leu⁺) by using a spheroplast transformation protocol (Hinnen et al., Proc. Natl. Acad. Sci. USA, 75:1929-33, 1978). The construction of strains 1260 and 1309 is provided in Example 8, supra.

Transformants were selected on synthetic agar medium lacking leucine and containing 1 M sorbitol. The top and bottom synthetic agar medium lacking leucine and containing 1 M sorbitol were obtained from REMEL, Lenexa, Kans. (cat #09459 and 92155, respectively). Clonal Leu⁺ isolates were obtained by serial growth on SD minus leucine plates (KD MEDICAL, Columbia Md.).

For screening multiple transformants, 5.0 mL production cultures were grown in culture tubes rotated slowly at 30° C. Subsequently, production was confirmed for selected transformants in 25-ml cultures in a 125-mL flask. For both cases, five-mL seed cultures were grown at 30° C. in 5× minus leucine medium containing 4.0% glucose and 0.1 M sorbitol for 18-24 hours to OD₆₀₀ of 1.5-3.0/mL. 5× minus leucine medium contains the following components per liter: Yeast Nitrogen Base without added amino acids or ammonium sulfate, 8.5 g; adenine, 0.40 g; L-tyrosine, 0.25 g; uracil, 0.20 g, succinic acid, 10.0 g, ammonium sulfate, 5.0 g and 50 ml of Leucine-Minus Solution #3. Leucine-Minus Solution #3 contains per liter of distilled water, L-arginine, 2 g; L-histidine, 1.0 g; L-isoleucine, 6 g; L-lysine 4.0 g; L-methionine, 1.0 g; L-phenylalanine, 6.0 g; L-tryptophan, 4.0 g. The pH of the medium was adjusted to 5.3 with 50% sodium hydroxide.

For production in tubes, a 0.3 mL aliquot of the seed culture was transferred to either 5.0 mL of 5× minus leucine medium containing 2% glucose, 4% galactose or YEHDG medium for 72 hours to a final OD₆₀₀ of 5-16.0/mL. YEHDG medium contains per liter: L-Hy-Soy peptone-Sheffield, 10 g; Yeast extract, 20 g; L-dextrose, 16 g; D(+) galactose, 40 g. For production in flasks, a 1.5-mL aliquot of the seed culture was transferred to 25-mL of medium and grown as described above with shaking at 220 rpm.

After harvesting 10 OD units per sample, the cell pellets were broken with glass beads in 0.3 mL lysis buffer (0.1 M sodium phosphate buffer, pH 7.2, 0.5 M NaCl, 2 mM PMSF). The lysate was recovered by centrifugation, the unbroken cells/beads were washed with 0.3 mL of lysate buffer and the clarified supernatants were combined. Protein concentration was determined by the BIO-RAD Protein Assay Dye Reagent system (BIO-RAD, Hercules, Calif.) according to the manufacturer's instructions. The cell lysates were analyzed for the expression of ORF0657n by immunoblot analysis after electrophoresis on 4-20% gradient Tris-Glycine gels (INVITROGEN, Carlsbad, Calif.) in 1× Tris-glycine SDS buffer (BIO-RAD) under reducing and denaturing conditions. The samples contained 20 μg of total cellular protein. The gels were electroblotted onto 0.45 micron nitrocellulose membrane filters (Optitran from Schleicher and Schuell, Keene, N.H.). To estimate protein size, prestained standards between 6.4 and 203 kDa (Broad Range Prestained SDS-PAGE Standard, BIO-RAD) were run in parallel with the lysates.

E. coli Standard

His-Tag ORF0657n (SEQ ID NO: 28) purified from the E. coli producing culture (E. coli host HMS174(DE3), NOVAGEN, Madison, Wis.), induced to produce ORF0657n, and a cell lysate were employed as standards. E. coli was transformed with an expression vector expressing His-Tag ORF0657n. The E. coli culture was grown overnight in LB broth containing 30 μg/mL kanamycin at 37° C. The next day, 60 μL of overnight culture was used to inoculate 6.0 mL LB plus 30 μg per mL kanamycin. The culture was grown at 37° C. for approximately 3 hours until the OD₆₀₀ was between 0.4-1.0. Expression was induced with 1 mM IPTG for 2 hours at 37° C. The cells were harvested and the cell pellet was stored at −80° C.

The E. coli lysate was prepared using Bugbuster Protein Extraction Reagent (NOVAGEN, Madison, Wis.) following the manufacturer's protocol. Proteins were immunodetected by Western blot using a murine monoclonal antibody (“designated “2H2B8”) to ORF0657n as primary antibody and goat anti-mouse IgG (H+L) horseradish peroxidase-linked whole antibody (ZYMED LABORATORIES, South San Francisco, Calif.) as the secondary antibody. Mab 2H2B8 was generated by immunization of mice with purified E. coli produced full-length ORF0657n. Mab 2H2B8 was selected by ELISA and was shown to be specific for ORF0657n. The filters were processed using the BIO-RAD HRP Conjugate Substrate Kit.

Expression Products from E. coli and S. cerevisiae

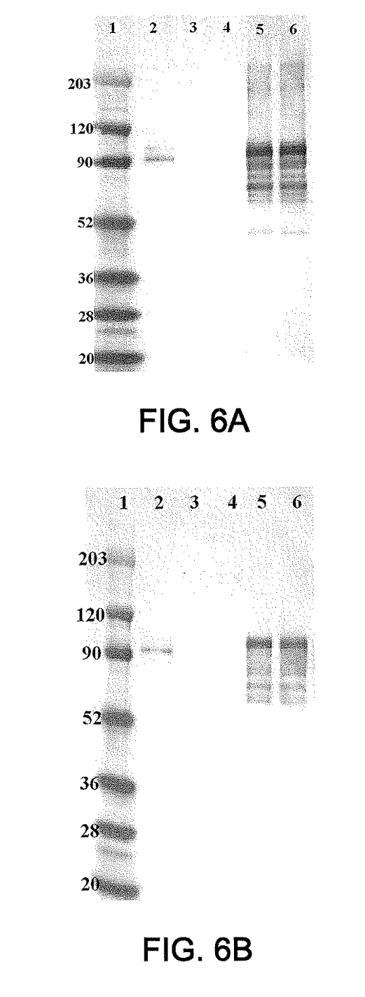

From the initial screening of yeast transformants, one transformant each of yeast strains 1260 and 1309 were chosen as the best producers of full-length ORF0657n region (SEQ ID NO: 28 without the carboxyl His-Tag). An example of their production of full-length ORF0657n region after a 72 hour fermentation in YEHDG medium compared to ORF0657n region (SEQ ID NO: 28) production in E. coli is shown in FIGS. 6A and 6B.

The major protein detected by Western blot analysis with mAb 2H2B8 was ˜105-110-kDa as shown in FIGS. 6A (strain 1260) and 6B (strain 1309): lanes 5 and 6. The ˜105-110-kDa protein corresponded in size to the largest protein detected in the sample of purified recombinant ORF0657n produced in E. coli (SEQ ID NO: 28; lane 2) or an extract of the induced E. coli ORF0657n-producing culture (the latter not included in gels shown in FIG. 6). The 105-110-kDa E. coli and S. cerevisiae produced proteins are of higher molecular weight than the predicted size in our gel-electrophoresis system. It should be noted that the predominant species detected in the E. coli controls was smaller, ˜95 kDa and comigrated with a minor amount of protein produced in yeast. The ˜105-110 kDa protein is believed to correspond to the full-length ORF0657n that was expressed with the E. coli secretory leader in both E. coli and in S. cerevisiae, and the 95-kDa protein is thought to be a degradation product. No detection was observed with an extract of a control transformant obtained with vector pGAL110 alone (lanes 3 and 4).

An estimate of the amount of ORF0657n region (SEQ ID NO: 28 without the carboxyl His-Tag) produced by the transformants of 1260 and 1309 was ˜10 micrograms ORF0657n/mL YEHDG medium with ORF0657n region comprising ˜2% of the total protein as determined by semi-quantitative Western blot with 100 ng of purified ORF0657n (SEQ ID NO: 28) as standard. Both the amounts expressed and % ORF0657n region (SEQ ID NO: 28 without the carboxyl His-Tag) of the total protein were greater at 72 hours compared to 48 hours in both strains (data not presented). The titer of ORF0657n region (SEQ ID NO: 28 without the carboxyl His-Tag) produced by transformants of 1260 in 5× leucine medium containing 2% glucose, 4% galactose, was ˜8.0 μg/mL and the % total protein was 2.0

### Example 10

### Expression of Secreted Codon-Optimized Sequence Encoding an ORF0657nH Region

Vector pKH4-3B (Carty et al., Biotechnol. Lett. 12:879-884, 1990) contains the yeast alpha factor (MFα1) pre-pro secretory leader. Fusion of a desired protein to this leader results in a translation product that is cleaved first by signal peptidase of S. cerevisiae. Subsequently the Kex2 protease cleaves after the “Lys Arg residue” in the secretory leader and the mature protein is released. The S. cerevisiae inducible GAL10 promoter was used to drive protein expression

The vector pKH4-3B was used to express a codon optimized gene encoding an altered SEQ ID NO: 3. SEQ ID NO: 3 was altered by removing the amino terminus methionine and adding an amino terminus leader sequence. The codon optimized SEQ ID NO: 3 encoding region is provided by nucleotides 4-1710 of SEQ ID NO: 32, and the leader encoding sequence is provided by vector pKH4-3B prepared as described below.

An in frame fusion of the alpha factor pre-pro secretory leader of pKH4-3B to ORF0657nH region of SEQ ID NO: 3 was made. This was accomplished by digestion of vector pKH4-3B with SphI followed by treatment with T4 DNA polymerase to remove the SphI-overhang, creating a blunt end at the 5′ end with the appropriate codon of the leader. Subsequently, the vector was digested with BamHI to create a 3′ cloning site. This construct requires a PCR product with a 5′ blunt end corresponding to the second codon of rebuilt ORF0657nH region (SEQ ID NO: 32), and a 3′ end with a stop codon and BamHI restriction site.

DNA encoding the ORF0657nH region with codons optimized for yeast expression was amplified from pUnkCR1 (Example 7) using the following sense and antisense primers, respectively, UCKHS2 (SEQ ID NO: 98), 5′ GCTGAAGAAACTGGTGGTACCAAC3′ and UCKHA2, (SEQ ID NO: 99) 5′GTCACGGATCCTTAAGACTTAGCCTTGTTTTCTTGAGTGTTC3′. The 5′ end of the sense primer, GCT, encodes alanine, the predicted N-terminus of the ORF0657nH region. The antisense primer contains a TAA stop codon and a BamHI site (underlined). The resulting 1.7-kb PCR product was gel isolated and cloned into pKH4-3B (prepared as described above) to construct pUS38. The entire DNA sequence of the insert and partial sequence of the vector was verified, and showed that the desired fusion to the secretory leader was made. Appropriate cleavage by Kex2p of the initial protein expressed from this construct would result in a protein corresponding to SEQ ID NO: 3 lacking the N-terminal methionine.

Plasmid pUS38 was used to transform S. cerevisiae strains 1260 and 1309 to leucine prototrophy (Leu⁺) by using a spheroplast transformation protocol (Hinnen et al., Proc. Natl. Acad. Sci. U.S.A. 75:1929-33, 1978). Transformants were selected on synthetic agar medium lacking leucine and containing 1 M sorbitol. The top and bottom synthetic agar medium lacking leucine and containing 1 M sorbitol were obtained from REMEL, Lenexa, Kans. (cat #09459 and 92155, respectively). Clonal Leu⁺ isolates were obtained by serial growth on SD minus leucine plates (KD MEDICAL, Columbia Md.).

For screening multiple transformants, five-mL seed cultures were grown at 30° C. in 5× minus leucine medium containing 4.0% glucose and 0.1 M sorbitol medium for 18-24 hours to OD₆₀₀ of 1.5-3.0/mL. 5× minus leucine medium contains the following components per liter: Yeast Nitrogen Base w/o amino acids or ammonium sulfate, 8.5 g; adenine, 0.40 g; L-tyrosine, 0.25 g; uracil, 0.20 g, succinic acid, 10.0 g, ammonium sulfate, 5.0 g and 50 mL of Leucine-Minus Solution #3, Leucine-Minus Solution #3 contains per liter of distilled water, L-arginine, 2 g; L-histidine, 1.0 g; L-isoleucine, 6 g; L-lysine 4.0 g; L-methionine, 1.0 g; L-phenylalanine, 6.0 g; L-tryptophan, 4.0 g. The pH of the medium was adjusted to 5.3 with 50% sodium hydroxide. A 0.3 mL aliquot was transferred to either 5.0 mL of 5× minus leucine medium containing 2% glucose plus 4% galactose or YEHDG medium for 72 hours to a final OD600 of 10-20.0/mL. YEHDG medium contains per liter: L-Hy-Soy peptone-Sheffield, 10 g; Yeast extract, 20 g; L-dextrose, 16 g; D(+) galactose, 40 g.

The fermentations were harvested by removing the yeast cells and analyzing the supernatants directly for the expression of ORF0657nH region (SEQ ID NO: 3) by Western blot analysis or by Coomassie staining of SDS-PAGE gels. For immunoblot analysis, 10 to 25 microliter samples were subjected to electrophoresis on 4-15% gradient Tris-HCl gels (BIORAD, Hercules, Calif.) in 1× Tris glycine SDS buffer (BIORAD, Hercules, Calif.) under reducing and denaturing conditions. The gels were electroblotted onto 0.2 micron PVDF membrane filters. Proteins were immunodetected with the monoclonal antibody 2H2B8 as primary antibody and goat anti-mouse IgG (H+L) horseradish peroxidase-linked whole antibody (ZYMED LABORATORIES, South San Francisco, Calif.) as the secondary antibody. Mab 2H2B8 is described in Example 9. The filters were processed using the WESTERN LIGHTNING™ Chemiluminesence Reagent Plus kit (PERKIN ELMER, Wellesley, Mass.).

For analysis of expression of recombinant yeast ORF0657nH region (SEQ ID NO: 3) by Coomassie staining of SDS-PAGE gels, samples were subjected to electrophoresis on 4-15% gradient Tris-HCl gels (BIO-RAD) in 1× Tris glycine SDS buffer (BIO-RAD) under reducing and denaturing conditions. The gels were stained with Bio-Safe Coomassie, a Coomassie G250 stain according to the manufacturer's protocol (BIO-RAD).

As a qualitative standard, a cell lysate from the E. coli culture producing ORF0657nH (SEQ ID NO: 4 with a carboxyl His-Tag) was employed. The predicted amino acid sequence of E. coli-expressed ORF0657nH region is identical to the predicted amino acid sequence of S. cerevisiae internally-expressed ORF0657nH region (SEQ ID NO: 3) except for the presence of glycine following the N-terminal methionine in the E. coli construct.

To produce ORF0657nH region in E. coli, the producing culture was grown overnight in LB broth containing 50 mg/mL kanamycin at 37° C. A pET28 encoding SEQ ID NO: 4 with a carboxyl His-Tag was used to obtain expression of protein. The next day, 500 μL of overnight culture was used to inoculate 5.0 mL LB broth plus 50 μg per mL kanamycin. The culture was grown at 37° C. for approximately 3 hours to an OD₆₀₀ of 0.6. Expression was induced with 1 mM IPTG for 3.5 hours at 37° C. The cells were harvested and the cell pellet was stored at −80° C. The E. coli lysate was prepared using Bugbuster Protein Extraction Reagent (NOVAGEN, Madison, Wis.) following the manufacturer's protocol.

Protein from a cell-lysate of S. cerevisiae transformant 1-1, expressing internal ORF0657nH region (SEQ ID NO: 3) described and prepared as indicated in Example 11 infra, was also included as a standard. To estimate protein size, prestained standards between 10 and 250 kDa (BIO-RAD) were run in parallel with the samples on SDS-PAGE gels for both Coomassie staining and Western evaluation.

Production of the desired species was obtained in 5× minus leucine medium containing 2% glucose plus 4% galactose. Transformants containing pUS38 of both strains 1260 and 1309 secreted an ˜80-kDa protein that comigrated very closely with yeast internally expressed ORF0657nH region (from nucleic acid encoding SEQ ID NO: 3) and E. coli expressed ORF0657nH (from nucleic acid encoding SEQ ID NO: 4 with a carboxyl His-Tag) that was detected by Western and Coomassie staining. In a typical experiment, 500 ng of these control lysates and 25 microliters of medium supernatant were subjected to electrophoresis. The detection was specific; the 80-kDa protein was not detected in a supernatant of a transformant containing vector alone by either Western blot or Coomassie staining. The secreted ˜80-kDa protein could correspond to mature non-glycosylated ORF0657nH region (SEQ ID NO: 3) or alternatively, it could contain a few glycosyl residues. A higher molecular weight species in the supernatants was detected by the antibody as well as two lower molecular weight proteins, all of which were stained by Coomassie Blue. The higher molecular weight species could contain unprocessed leader and/or could be glycosylated. The low MW species are likely to be degradation products.

An estimate of the 80-kDa species amount secreted by transformants of both strains 1260 and 1309 was ˜2 μg per mL culture medium. This was determined by comparing the Western signal at 80 kDa to that of the sample of the yeast cell lysate containing ORF0657nH region (SEQ ID NO: 3), suggesting that ORF0657nH region comprises at least 50% of the total protein. The combined titer of the remaining species of ORF0657n region was estimated to be approximately 50 μg per mL culture medium.

### Example 11

### Intracellular Expression of ORF0657nH Region (SEQ ID NO: 3) in S. cerevisiae

A very high level production of discretely-sized protein was obtained by intracellular expression of SEQ ID NO: 3 in S. cerevisiae. Expression was achieved using a yeast optimized encoding nucleic acid sequence.

DNA encoding ORF0657nH region with codons optimized for yeast expression (SEQ ID NO: 32) was amplified from pUnkCR1 (Example 7), using the following sense and antisense primers, respectively, (SEQ ID NO: 100) 5′GGGG GGATCC CACAAAACAAA ATG GCT GAA GAA ACT GGT 003′ and (SEQ ID NO: 101) 5′GGG GGG GGATCC TTA AGA CTT AGC CTT GTT TTC TTG AGT3′ with flanking BamHI restriction sites (underlined). The sense primer contains a 5′ untranslated leader sequence and ATG codon, and the antisense primer contains TAA as the stop codon.

The resulting 1.7-kb product was gel-isolated and cloned into the TOPO TA vector pCR2.1 (INVITROGEN CORPORATION, Carlsbad, Calif.), according to the manufacturer's directions, to construct plasmid pCR_iUC-S. The DNA sequence of the insert was subsequently determined from two independent clones, pCR_iUCS2.2 and pCR_iUCS-2.4. A single PCR error was found in each of the inserts, located on different HindIII fragments of each plasmid. A plasmid with the correct sequence was obtained by swapping the 1.3-kb HindIII fragment of pCR_iUC-S2.2 with the correct sequence, for the corresponding fragment of pCR_iUC-S2.4.

The 1.3-kb fragment of pCR_iUC-S2.2 and the 4.3-kb fragment of pCR_iUC-S2.4 were gel-isolated and ligated. A clone with the desired fragments was selected, pCR_iUC-S, and the appropriate orientation and nucleotide sequence verified by DNA sequencing. Subsequently, the 1.7-kb BamHI fragment was subcloned into pGAL110 (FIG. 5A) to construct piUCS-S(−).

Plasmid DNA from piUC-S(−) was used to transform S. cerevisiae strains 1260 and 1309 to leucine prototrophy (Leu⁺) by using a spheroplast transformation protocol. (Hinnen et al., Proc. Natl. Acad. Sci. USA, 75:1929-33, 1978.) Transformants were selected on synthetic agar medium lacking leucine and containing 1 M sorbitol. The top and bottom synthetic agar medium lacking leucine and containing 1 M sorbitol were obtained from REMEL, Lenexa, Kans. (cat #09459 and 92155, respectively). Clonal Leu⁺ isolates were obtained by serial growth on SD minus Leu plates (KD MEDICAL, Columbia Md.).

Multiple transformants were screened for production of ORF0657nH region using the fermentation conditions described in Example 9. The cell lysates were analyzed for the production of ORF0657nH region by Western blot analysis or by analysis of SDS-PAGE gels stained with Coomassie blue.

For Western blot analysis, samples were subjected to electrophoresis on 4-15% gradient Tris-HCl Criterion gels (BIO-RAD, Hercules, Calif.) in 1× Tris glycine SDS buffer (BIO-RAD) under reducing and denaturing conditions. The gels were electroblotted onto 0.2-micron PVDF membrane filters. Proteins were immunodetected by Western blot using a monoclonal antibody to full-length ORF0657n (SEQ ID NO: 28), 2H2B8, as primary antibody and goat anti-mouse IgG (H+L) horseradish peroxidase-linked whole antibody (ZYMED LABORATORIES, South San Francisco, Calif.) as the secondary antibody. Mab 2H2B8 was described in Example 9. The filters were processed using the WESTERN LIGHTNING™ Chemiluminesence Reagent Plus kit (PERKIN ELMER, Wellesley, Mass.).

For analysis of expression of recombinant yeast ORF0657nH (encoded by SEQ ID NO: 32) by Coomassie staining of SDS-PAGE gels, samples were subjected to electrophoresis on 4-15% gradient Tris-HCl Criterion gels (BIO-RAD) in 1× Tris glycine SDS buffer (BIO-RAD) under reducing and denaturing conditions. The gels were stained with Bio-Safe Coomassie, a Coomassie G250 stain (BIO-RAD) according to the manufacturer's protocol.

The samples of yeast cell-lysate subjected to electrophoresis contained 0.5 and 1.25 μg of total cellular yeast protein, respectively, for Western blot and Coomassie-stain. As a quantitative standard for the Coomassie staining, purified BSA (100×, NEW ENGLAND BIOLABS, Beverly, Mass.) was used. Purified E. coli full-length recombinant ORF0657n region (SEQ ID NO: 28) was used as a quantitative standard for the Westerns. As a qualitative standard, a cell lysate from the E. coli culture producing ORF0657nH region (SEQ ID NO: 4 with a carboxyl His-Tag), induced to produce ORF0657nH, was employed for both evaluations.

To produce ORF0657nH region (SEQ ID NO: 4 plus a carboxyl His-Tag) in E. coli, the producing culture was grown overnight in LB broth containing 50 μg/mL kanamycin at 37° C. The next day, 500 μl of overnight culture was used to inoculate 5.0 mL LB broth plus 50 μg per mL kanamycin. The culture was grown at 37° C. for approximately 3 hours to an OD₆₀₀ of 0.6. Expression was induced with 1 mM IPTG for 3.5 hours at 37° C. The cells were harvested and the cell pellets were store at −80° C. The E. coli lysate was prepared using Bugbuster Protein Extraction Reagent (NOVAGEN, Madison, Wis.) following the manufacturer's protocol. To estimate protein size, prestained standards between 10 and 250 kDa were run in parallel with the lysates (BIO-RAD).

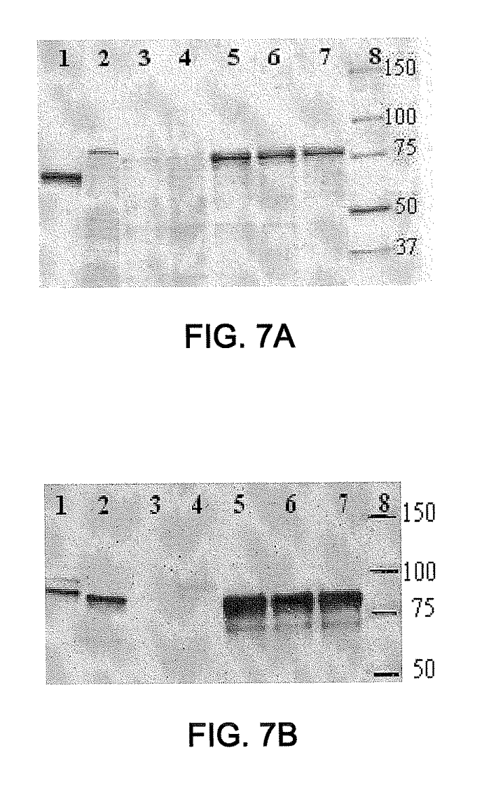

From the initial screening, one transformant of yeast strain 1260, designated 1-1, was chosen as the best producer of ORF0657nH region (SEQ ID NO: 3). An example of the production of ORF0657nH region after a 72 hour fermentation in complex medium YEHDG in shake flasks is shown in FIGS. 7A and 7B. The major protein detected by either Coomassie staining (A) or by the antibody (B) was ˜85 kDa (see lanes 5, 6, and 7) and comigrated with E. coli ORF0657nH (SEQ ID NO: 4 with a carboxyl His-Tag; lane 2). The MW of both the E. coli expressed ORF0657n and the yeast-expressed ORF0657n was larger than the predicted size in our gel electrophoresis system. Detection of the ˜85-kDa protein was specific; no detection was observed in a cell lysate from a transformant containing vector pGAL110 alone (lane 3). Significantly more ORF0657nH region (SEQ ID NO: 3) was produced compared to full-length ORF0657n region (SEQ ID NO: 28 without a carboxyl His-Tag), compare lanes 5, 6, and 7 to lane 4. The transformant containing full-length ORF0657n was fermented at the same time as the transformant containing ORF0657nH region (SEQ ID NO: 3) and under the identical conditions. Little evidence of degradation of mature yeast-expressed ORF0657nH region was observed.

To evaluate protein stability, a sample from a cell lysate of transformant 1-1 that had been frozen for several days and subsequently unthawed was included on the gel (FIG. 7, lane 7). It can be seen that the integrity and amount of this sample is similar to the fresh samples (lanes 5 and 6). The lack of degradation and stability of the ORF0657nH region (SEQ ID NO: 3) was confirmed by Western blot using a goat polyclonal antibody raised against full-length E. coli ORF0657n (SEQ ID NO: 28) (data not shown).

An estimate of the amount of ORF0657nH region (SEQ ID NO: 3) from the Western blot and Coomassie gel was ˜360 μg/per mL YEHD medium and the % total protein was estimated to be ˜50. The amount produced in defined medium was 225 μg/mL culture medium and the % of the total protein was also ˜50. The amount of ORF0657nH region (SEQ ID NO: 3) compared to the amount of full-length ORF0657n region (SEQ ID NO: 28 without a carboxyl His-Tag) was higher (350 vs 10 μg/mL, respectively) and the % total protein was also higher (50 vs 2.0).

### Example 12

### Intracellular Expression of ORF0657nG Region (SEQ ID NO: 44) in S. cerevisiae

Intracellular expression of ORF0657n related polypeptides was evaluated in the following example using a DNA construct encoding ORF0657nG region (SEQ ID NO: 44). ORF0657nG (SEQ ID NO: 44) is a truncated version of SEQ ID NO: 2, that lacks the amino-terminal signal sequence. SEQ ID NO: 44 retains the N-terminal methionine and amino acids 42-645 of SEQ ID NO: 2.

Yeast optimized DNA encoding ORF0657nG region (SEQ ID NO: 44) was amplified from pUnkCR1 (Example 7), using the following sense and antisense primers, respectively, 5′GGGGGGATCCCACAAAACAAAATGGCTGAAGAAACTGGTGG3′ (SEQ ID NO: 102) and 5′GGGGGGGATCCTTAGTTCTTTCTCTTTCTTGG3′ (SEQ ID NO: 103) with flanking BamHI restriction sites. The sense primer contains a 5′ untranslated leader sequence and ATG codon, and the antisense primer contains TAA as the stop codon. The resulting 1.8-kb product was gel-isolated and cloned into the TOPO TA vector pCR2.1 according to the manufacturer's directions (INVITROGEN CORPORATION, Carlsbad, Calif.), to construct plasmid pCR_iUC-L4. DNA sequence analysis confirmed that a single deletion occurred at the 5′-end.

A clone with the correct sequence was obtained by swapping the 1.3-kb HindIII-HindIII fragment of pCR_iUC-S2.2 containing the correct sequence (see Example 11), for the corresponding fragment of pCR_iUC-L4. The 1.3-kb fragment of pCR_iUCS2.2 and the 4.4-kb fragment of pCR_iUC-L4 were gel-isolated and ligated. A clone with the desired fragments was selected, pCR_iUC-L, and the appropriate orientation and nucleotide sequence verified by DNA sequencing. Subsequently, the 1.8-kb BamHI fragment was subcloned into pGAL110 to construct piUC-L(−) and the DNA sequence was verified.

Plasmid DNA from piUC-L(−) was used to transform S. cerevisiae strain 1260 to leucine prototrophy (Leu⁺) as described in Example 9. Several yeast transformants were screened for intracellular production of ORF0657nG region (SEQ ID NO: 44) using the conditions for fermentation and cell breakage described in Example 11. 0.25-0.5 μg of cell-lysate protein were analyzed for the production of ORF0657nG region by Western blot analysis as described in Example 11. Purified E. coli ORF0657nH region (SEQ ID NO: 4 with a carboxyl His-Tag) was used as a quantitative standard and a cell lysate from the E. coli culture producing ORF0657nG region (SEQ ID NO: 44 with a carboxyl His-Tag), induced to produce ORF0657nG region, was used as a qualitative standard. The samples were subjected to electrophoresis on 4-15% gradient Tris-HCl Criterion gels (BIO-RAD, Hercules, Calif.) in 1× Tris glycine SDS buffer (BIO-RAD) under reducing and denaturing conditions. The gels were electroblotted onto 0.2-micron PVDF membrane filters (BIO-RAD).

Proteins were immunodetected by Western blot using a polyclonal antibody to purified E. coli produced full-length ORF0657n (SEQ ID NO: 28 without the carboxyl His-Tag) as primary antibody and goat anti-mouse IgG (H+L) horseradish peroxidase-linked whole antibody (ZYMED LABORATORIES, South San Francisco, Calif.) as the secondary antibody. The polyclonal antibody was generated by immunization with purified E. coli-produced full-length ORF0657n region (SEQ ID NO: 28). The filters were processed using the WESTERN LIGHTNING™ Chemiluminesence Reagent Plus kit (PERKIN ELMER, Wellesley, Mass.).

For comparison purposes, a ORF0657nG region (SEQ ID NO: 44 with a carboxyl His-Tag) was cloned in an E. coli expression vector as described in Example 1 and expressed. To produce the ORF0657nG region in E. coli, the producing culture was grown overnight in LB broth containing 50 μg/mL kanamycin at 37° C. The next day, 500 μL of overnight culture was used to inoculate 5.0 mL LB broth plus 50 μg per mL kanamycin. The culture was grown at 37° C. for approximately 3 hours to an OD₆₀₀ of 0.6. Expression was induced with 1 mM IPTG for 3.5 hours at 37° C. The cells were harvested and the cell pellets were stored at −80° C. The E. coli lysate was prepared using Bugbuster Protein Extraction Reagent following the manufacturer's protocol (NOVAGEN, Madison, Wis.). To estimate protein size, prestained standards between 10 and 250 kDa (BIO-RAD) were run in parallel with the lysates.

From the initial screening, one transformant of yeast strain 1260, designated iUC-L3, was chosen as a good producer of ORF0657nG region after a 72 hour fermentation in complex medium YEHDG in culture tubes as described in Example 11. The major protein detected by either Coomassie staining or by the antibody was ˜85 kDa and comigrated with E. coli expressed ORF0657nG region (SEQ ID NO: 44 with a carboxyl His-Tag). The MW of both the E. coli expressed ORF0657nG region and the yeast-expressed ORF0657nG region was larger than the predicted size in our gel electrophoresis system. Detection of the ˜85-kDa protein was specific; no detection was observed in a cell lysate from a transformant containing vector pGAL110 alone. The average titer of ORF0657nG determined from three Western blot experiments was 30 μg/per mL of YEHDG medium and the % total protein was estimated to be 10.0 (see Example 13 infra).

### Example 13

### Removing the Cell Wall Sorting Signal Increases Intracellular Expression

The effect of removing the ORF0657n carboxyl cell sortase C-terminal encoding region alone, and in combination with removing the N-terminal signal region was examined using codon-optimized ORFO657n genes SEQ ID NOs: 31, 32, and 45. SEQ ID NO: 31 encodes full-length ORF0657n region. SEQ ID NO: 32 encodes ORF0657nH region, which lacks both an N-terminal signal sequence and C-terminal cell wall sorting region. SEQ ID NO: 45 encodes ORF0657nG region lacking just the signal peptide sequence.

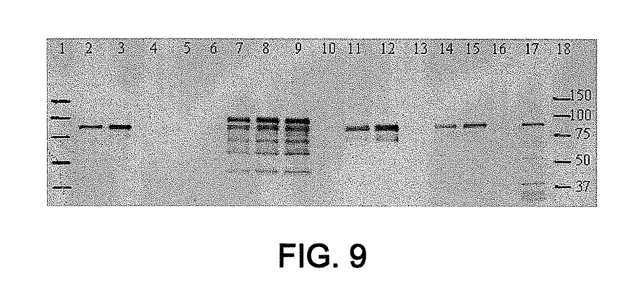

One transformant each expressing full-length ORF0657n region (SEQ ID NO: 28 without the carboxyl His-Tag; transformant rUnkC1), ORF0657nH region (SEQ ID NO: 3; transformant 1-1), and ORF0657nG region (SEQ ID NO: 44 transformant iUC-L3) was fermented, cell lysates were prepared and analyzed as described in Example 11. A representative Western blot is shown in FIG. 9. The amount of protein cell lysate loaded on the gel from the different transformants was not the same in order to compensate for the differing levels of expression of the three constructs encoding ORF0657n regions. In each case, at least two different amounts of protein cell lysate from each ORF0657n-containing transformant were loaded.

Typical results demonstrating that ORF0657nH region (SEQ ID NO: 3) was expressed significantly better than full-length ORF0657n region (SEQ ID NO: 28 without the carboxyl His-Tag) or ORF0657nG region (SEQ ID NO: 44) are shown in FIG. 9. A substantial signal was detected with only 50 and 100 ng cell lysate protein from the ORF0657nH region-producer as shown in lanes 11 and 12. In contrast, 1.0 and 2.0 μg cell lysate protein of the full-length ORF0657nC region-producer, lanes 7 and 8, respectively, was used. A good signal was detected for ORF0657nG region with less protein, 250 and 500 ng protein were loaded as shown in lanes 14 and 15.

Table 4 presents a comparison of the average titer of each of the three ORFs and % total protein determined from three independent Western blots. Cell lysates were prepared fresh for each Western blot. The averages include results from two independent fermentations. The amount of specific ORF0657n was determined relative to the purified E. coli ORF0657nH (SEQ ID NO: 4 plus a carboxyl His-Tag) standard.

TABLE 4

| ORF0657n Produced | Titer, μg/mL YEHDG Medium | % Total Protein |
|---|---|---|
| C | 10.0 | 4.0 |
| G | 30.0 | 10.0 |
| H | 425.0 | 80.0 |

Removal of the signal peptide sequence enhanced % total protein and titer 2.5-3-fold as determined by comparing expression of full-length ORF0657n region (SEQ ID NO: 28 without the carboxyl His-Tag) and ORF0657nG region (SEQ ID NO: 44). Removal of both the signal peptide and cell wall sorting sequence enhanced expression 8-14-fold based compared to removal of just the signal peptide sequence (compare G and H region), and enhanced expression 20-42.5 fold compared to the full length protein (C to H region). The magnitude of the increase in expression upon removal of the cell wall sorting sequence was not expected.

### Example 14

### Intracellular Expression of ORF0657nI Region in S. cerevisiae

DNA encoding the ORF0657nI region with codons optimized for yeast expression (SEQ ID NO: 33) was amplified from pUnkCR1 (Example 7), using the following sense and antisense primers, respectively, 5′CTCCGGATCCCACAAAACAAAATGGCTGAAGAAACTGGT 3′ (SEQ ID NO: 104) and 5′GCTGCCGGGATCCTTATGGGGTTGGCTTAGATGGGGTAG 3′ (SEQ ID NO: 105) with flanking BamHI restriction sites (underlined). The sense primer contains a 5′ untranslated leader sequence and ATG codon, and the antisense primer contains TAA as the stop codon. The resulting 1.3-kb product was gel-isolated and cloned into pGAL110 (FIG. 5A) to construct piUC-I (FIG. 5B) and the DNA sequence was verified.

Plasmid DNA from piUC-I was used to transform S. cerevisiae strain 1260 to leucine prototrophy (Leu⁺) as described in Example 9. Three Leu⁺ transformants were screened for intracellular production of ORF0657nI region (SEQ ID NO: 1) using the small-scale conditions for fermentation (5.0 mL cultures) as described in Example 9.

Production was compared to that of ORF0657nH region (SEQ ID NO: 3) produced by S. cerevisiae transformant 1-1 (see Example 11). Cell lysates were prepared as described in Example 9, and 100 and 200 ng of yeast cell-lysate protein were analyzed for the production of ORF0657nI and ORF0657nH by semi-quantitative Western blot analysis as described in Example 11. Purified E. coli ORF0657nH (SEQ ID NO: 4 with a carboxyl His-Tag) was used as a quantitative standard. The samples were subjected to electrophoresis on 10-20% gradient Tris-HCl Criterion gels (BIO-RAD, Hercules, Calif.) in 1× Tris glycine SDS buffer (BIO-RAD) under reducing and denaturing conditions. The gels were electroblotted onto 0.2-micron PVDF membrane filters (BIO-RAD). Proteins were immunodetected by Western blot using a polyclonal antibody to purified E. coli full-length ORF0657n (SEQ ID NO: 28) as primary antibody and goat anti-mouse IgG (H+L) horseradish peroxidase-linked whole antibody (ZYMED LABORATORIES, South San Francisco, Calif.) as the secondary antibody. The polyclonal antibody was generated by immunization with purified E. coli-produced full-length ORF0657n (SEQ ID NO: 28). The filters were processed using the WESTERN LIGHTNING™ Chemiluminesence Reagent Plus kit (PERKIN ELMER, Wellesley, Mass.). To estimate protein size, prestained standards between 10 and 250 kDa (BIO-RAD) were run in parallel with the lysates.

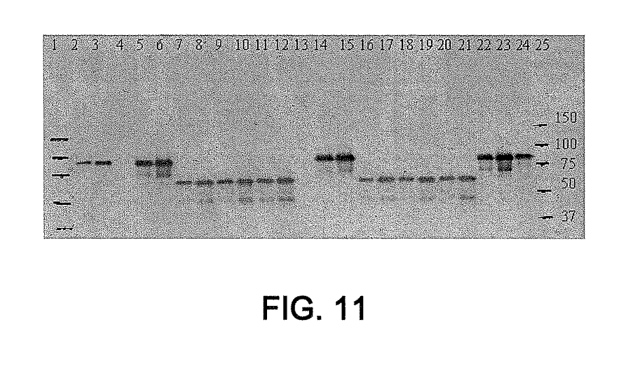

FIG. 11 presents results from a 72 hour fermentation in complex YEHDG medium in culture tubes. Strong detection of yeast-expressed ORF0657nH region (SEQ ID NO: 3) was obtained as shown in lanes 5, 6, 14, 15, 21 and 22. An ˜60-kDa protein in the transformants constructed to express ORF0657nI (SEQ ID NO: 1) was also readily detected as shown in lanes 7-12 and 16-21. Yeast-expressed ORF0657nI (SEQ ID NO: 1) comigrated with ORF0657nI (SEQ ID NO: 5 with a carboxyl His-Tag) expressed and purified from E. coli (data not presented). The MW of both the E. coli expressed ORF0657nI region and the yeast-expressed ORF0657nI region was larger than the predicted size in our gel electrophoresis system. The 60-kDa protein detected in transformants constructed to express ORF0657nI region (SEQ ID NO: 1) was specific; no protein was detected in a cell lysate from a vector control transformant (lanes 4 and 13).

The amount of ORF0657nH region (SEQ ID NO: 3) and ORF0657nI region (SEQ ID NO: 1) on the gel was estimated from the semi-quantitative Western blot by comparison with a known amount of purified ORF0657nH (SEQ ID NO: 4 with a carboxyl His-Tag). The titers and percent total protein were calculated as the average determined from the duplicate lysates containing the two different amounts of protein on the gel. The volumetric titers of ORF0657nH region (SEQ ID NO: 3) determined from a freshly fermented sample and a frozen cell-lysate were comparable, ˜500 and ˜550 μg/mL culture medium, and 320 μg ORF0657nI region (SEQ ID NO: 1) was produced per mL culture medium (the titer for ORF0657nI region is for transformant I1). The percent ORF0657nH region (SEQ ID NO: 3) of total protein was estimated as 78% and 80%, respectively, while ORF0657nI (SEQ ID NO: 1) comprised 45% of the total protein.

Thus, ORF0657nI region expressed well in S. cerevisiae with a titer about 1.5-fold lower than that of ORF0657nH (SEQ ID NO: 3) and the % total protein was about 1.3-fold lower. Good expression of ORF0657nH region in yeast was confirmed by Coomassie staining of an SDS-PAGE gel containing the cell lysates. Expression of ORF0657n′ region in YEDHG medium was scalable: production in either 125 mL or 2 L shake flasks was comparable to small-scale expression in culture tubes.

ORF0657nI region (SEQ ID NO: 1) also expressed well in defined 5× minus leucine medium (medium described in example 9). The titer was only ˜1.5-fold lower than the titer in complex medium YEHDG whereas the % total protein was comparable in both media. Integrity of ORF0657nI was good in both media with no significant degradation detected. Production of ORF0657nI was higher at 72 than 96 hours in both complex and 5× minus leucine medium, when tested in shake flask fermentations.

### Example 15

### Large-Scale Fermentation of Yeast Strain Producing ORF0657nH (SEQ ID NO: 3)

Frozen seed stock of yeast strain 1-1 (strain 1260 transformed with plasmid piUC-S(−); described in Example 11) was used for large-scale fermentation and purification. The vial of seed stock was thawed and 1.0 mL was used to inoculate a 250-mL Erlenmeyer flask containing 50 mL of a leucine-free selective medium (5× Leu⁻ medium, Bayne et al., Gene 66(2):235-44, 1988) containing 4% glucose. The flask was incubated at 28° C., 250 rpm on a rotary shaker. After 24 hours (residual glucose 215 g/L), a culture volume of 13 mL was added to a 2-L flask containing 877 mL of the same medium. Again, the flask was incubated at 28° C. and agitated at 250 rpm. After 24 hours (residual glucose 4.04 g/L), the contents of the 2-L flask were used to inoculate a 20-L reactor containing a chemically defined medium (Oura, Biotechnol. Bioengineer. 16:1197, 1974) that was optimized for the employed strains. This medium contained 20 g/L glucose followed by 25 g/L galactose for induction. The reactor was operated at 28° C., 4.7 L/min, 15 psig, and 300 rpm. Under these conditions, dissolved oxygen levels were maintained at greater than 30% of saturation. Cellular growth was monitored by glucose consumption, optical density (A₆₀₀ nm, 1-cm cuvettes), dry cell weight, galactose utilization and ethanol production. Cultivation continued for 90 hours reaching an A₆₀₀ of 33.9 and a dry cell weight of 17.5 g/L.

The culture was harvested via hollow fiber tangential flow filtration (AMICON H5MP01-43 cartridge) using an AMICON DC-10 harvest skid (MILLIPORE, Billerica, Mass.). The permeate was discarded and the cells were concentrated, diafiltered with PBS, and collected by centrifugation at 8000 rpm, 4° C. for 20 minutes using a Sorvall Evolution RC (SLA-3000 rotor). Cells were stored at −70° C.

To evaluate production of ORF0657nH by strain 1-1 in large-scale fermentation, cell lysates were prepared from 10 OD₆₀₀ units of the harvested culture and evaluated as detailed in Example 11 (results not shown). Production of ORF0657nH from a shake flask fermentation (72 hour, complex YEHDG medium) was also assessed. The results of a Western blot analysis (performed as described in Example 11) demonstrated that the protein produced from the large-scale fermentation comigrated with ORF0657nH produced in a shake flask fermentation (results not shown). The titer of ORF0657nH from the large-scale (20-L) fermentation was estimated to be 739 μg per mL and the % total protein was estimated to be 55%, compared to 732 μg/mL and 58% of the total protein for the shake flask fermentation (using the semi-quantitative Western method described in Example 11). These results verify that the production of ORF0657n can be scaled up in yeast.

### Example 16

### Protective Immunity of ORF0657n Related Polypeptides Produced in Yeast

The ability of yeast produced ORF0657n related polypeptides to provide protective immunity was evaluated by expressing a polypeptide of SEQ ID NO: 3 in yeast. E coli expressed full-length ORF0657nC (SEQ ID NO: 28), ORF0657nH (SEQ ID NO: 4 with carboxyl-terminal His-Tag), ORF0657nI (SEQ ID NO: 5 with carboxyl-terminal His-Tag), and adjuvant alone were used as controls.

An ORF0657n related polypeptide of SEQ ID NO: 3 was obtained from yeast and used in an animal model to provide protective immunity. The polypeptide of SEQ ID NO: 3 was expressed as described in Example 11.

Frozen recombinant S. cerevisiae cells expressing ORF657nH (SEQ ID NO: 3) were resuspended at 5 mL per gram wet cell weight in 0.2 M MOPS, pH 7.0 with protease inhibitors (EDTA free). A lysate was prepared by four passes through a microfluidizer at 14,000 psi (Microfluidics Model 110S). The Lysate was clarified by centrifugation (10,000×g, 20 minutes, 2-8° C.) followed by coarse filtration (glass fiber pre-filter, Millipore) and fine filtration (0.2 μm cellulose acetate, Whatman).

The Clarified Lysate was fractionated on a size-exclusion chromatography (SEC) column (Pharmacia HiPrep 26/60 Sephacryl S-300 HR, mobile phase: 0.2 M MOPS, pH 7.0). Fractions were analyzed by SDS-PAGE with Coomassie detection and Western blotting using ORF0657n protein specific anti-serum (raised against full-length ORF0657n, SEQ ID NO: 28) Fractions that contained product were pooled.

The SEC Product was sterile filtered through 0.2 μm cellulose acetate under aseptic conditions. Purity of the Sterile-Filtered Product was ≧ or higher and 94% by SDS-PAGE and Western blot. Sterile-Filtered Product was formulated at 0.2 mg/ml with AHP and Thimerosal.

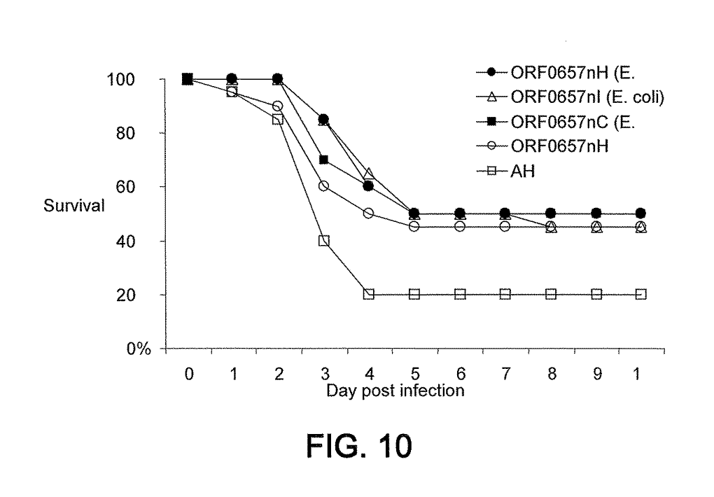

The ability of ORF0657n related polypeptides produced in yeast to provide protective immunity is illustrated in FIG. 10. Yeast expressed ORF0657nH (SEQ ID NO: 3) provided equivalent protective immunity as E. coli expressed polypeptides.

### Example 17

### Anamnestic Response in Non-Human Primates

Three groups of Rhesus monkeys were immunized with either yeast produced ORF0657n related polypeptide (ORF0657nH, SEQ ID NO: 3) or E coli-expressed ORF0657 related polypeptide (full-length ORF0657n, SEQ ID NO: 28) formulated with or without AHP. The monkeys in the vaccine group received 50 mcg ORF0657n related polypeptides via the intramuscular route.

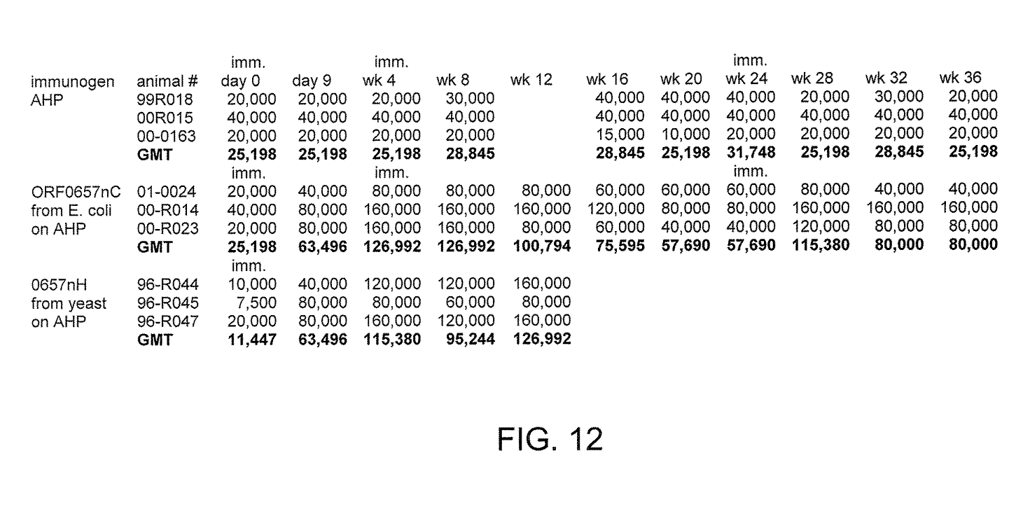

As shown in FIG. 12, the vaccine group animals responded, after a single dose, with a ˜3- to 6-fold increase in geometric mean titers over modest pre-existing titers, suggesting an anamnestic response. By contrast, geometric mean titers for animals in the control group remained the same (within the variability of the antibody assay). After a second dose of vaccine, geometric mean post-dose one titers in the vaccine and control groups changed very little compared to the post-dose titers.

To observe the development of antibody responses after only one dose, the group receiving yeast-expressed ORF0657H region (SEQ ID NO: 3) was followed out to 3 months (last time point tested). Antibody titers continued to rise after 9 days reaching levels that were not surpassed even after 2 or 3 doses of vaccine.

These observations suggest that one dose of the vaccine can elicit substantial and lasting antibody responses subsequent to natural priming of the immune system likely due to environmental exposure to S. aureus. A survey of pre-existing antibody titers in humans demonstrated the presence of modest antibody titers against ORF0657n in all samples tested (data not shown).

Other embodiments are within the following claims. While several embodiments have been shown and described, various modifications may be made without departing from the spirit and scope of the present invention.

1-19. (canceled)

20. A method of inducing a protective immune response in a patient comprising the step of administering to said patient an immunologically effective amount of a purified polypeptide immunogen, consisting of either (a) an amino acid sequence at least 94% identical to SEQ ID NO:3, or (b) a fragment of said amino acid sequence at least 94% identical to SEQ ID NO:3, wherein said fragment comprises an amino acid sequence at least 94% identical to SEQ ID NO: 1.

21. (canceled)

22. The method of claim 20, wherein said patient is a human.

23. The method of claim 22, wherein said patient is treated prophylactically against S. aureus infection.

24. A method of inducing a protective immune response in a patient comprising the step of administering to said patient an immunologically effective amount of a polypeptide made by a method comprising the steps of:
(a) growing a recombinant cell comprising a nucleic acid encoding a polypeptide immunogen under conditions wherein a polypeptide is expressed; wherein the polypeptide immunogen consists of either (a) an amino acid sequence at least 94% identical to SEQ ID NO:3, or (b) a fragment of said amino acid sequence at least 94% identical to SEQ ID NO:3, wherein said fragment comprises an amino acid sequence at least 94% identical to SEQ ID NO: 1 and wherein said polypeptide immunogen provides protective immunity against S. aureus; and
(b) purifying the polypeptide.

25. A method of inducing an anamnestic response in a patient comprising the step of administering to said patient an effective amount of an immunogen comprising a polypeptide, wherein said polypeptide comprises an amino acid sequence at least 94% identical to SEQ ID NO: 1 and provides protective immunity against S. aureus.

26. The method of claim 25, wherein said anamnestic response results in at least a 3-fold increase in geometric titer over pre-existing titer within 3 days.

27-32. (canceled)

33. The method of claim 20, wherein said polypeptide immunogen consists of an amino acid sequence at least 94% identical to SEQ ID NO:1, SEQ ID NO:3, or SEQ ID NO:42.

34. The method of claim 20, wherein said polypeptide immunogen consists of the amino acid sequence of SEQ ID NO: 1, 3, 7, 17, 20, or 42, each with up to 20 additional amino acids, wherein the up to 20 additional amino acids can be located at the carboxyl or the amino terminus.

35. The method of claim 34, wherein the polypeptide immunogen consists of the amino acid sequence of SEQ ID NO:1, 3, 7, 17, 20, or 42.

36. The method of claim 20, wherein said polypeptide immunogen is SEQ ID NO:1 or differs from SEQ ID NO:1 by up to 25 amino acid alterations.

37. The method of claim 36, wherein said polypeptide immunogen is SEQ ID NO:1 or differs from SEQ ID NO:1 by up to 10 amino acid alterations

38. The method of claim 37, wherein said polypeptide immunogen is SEQ ID NO:1 or differs from SEQ ID NO:1 by up to 5 amino acid alterations

39. The method of claim 38, wherein said polypeptide immunogen is SEQ ID NO:1.

40. The method of claim 25, wherein the polypeptide immunogen consists of an amino acid sequence at least 90% identical to SEQ ID NO:1 and one or more additional regions or moieties covalently joined to said sequence at the carboxyl terminus or the amino terminus, wherein each of said one or more additional regions or moieties is independently selected from a region or moiety having at least one of the following properties: enhances the immune response, facilitates purification, or facilitates polypeptide stability.
